(19) United States
(12) Patent Application Publication
(10) Pub. No.: US 2025/0262215 A1
(43) Pub. Date: Aug. 21, 2025

(54) USE OF CYCLIN E1 STATUS AS A PREDICTIVE BIOMARKER FOR TREATING CANCER WITH WEE1 INHIBITORS

(71) Applicant: Zeno Management, Inc., San Diego, CA (US)

(72) Inventors: Heekyung Chung, San Diego, CA (US); Olivier Harismendy, San Diego, CA (US); Jianhui Ma, San Diego, CA (US); Doris Sujung Kim, Berkeley, CA (US); Mark Lackner, San Mateo, CA (US); Kimberly Blackwell, Indianapolis, IN (US); Fernado Donate, Santa Monica, CA (US); Petrus Rudolf de Jong, San Diego, CA (US); Laure Escoubet, San Diego, CA (US); Peter Qinhua Huang, San Diego, CA (US); Kevin Duane Bunker, Escondido, CA (US)

(21) Appl. No.: 19/201,446

(22) Filed: May 7, 2025

Related U.S. Application Data
(63) Continuation of application No. PCT/US2023/078809, filed on Nov. 6, 2023.
(60) Provisional application No. 63/588,235, filed on Oct. 5, 2023.
(60) Provisional application No. 63/506,023, filed on Jun. 2, 2023.
(60) Provisional application No. 63/504,166, filed on May 24, 2023.
(60) Provisional application No. 63/459,520, filed on Apr. 14, 2023.
(60) Provisional application No. 63/485,764, filed on Feb. 17, 2023.
(60) Provisional application No. 63/382,817, filed on Nov. 8, 2022.

(51) Int. Cl. A61K 31/52; A61K 31/337; A61K 31/7068; A61P 35/00; G01N 33/574

(52) U.S. Cl. CPC A61K 31/52; A61K 31/337; A61K 31/7068; A61P 35/00; G01N 33/574; G01N 2333/4739

(57) ABSTRACT

The present disclosure provides, among other things, methods for treating cancer comprising administering an effective dose of Azenosertib to subjects selected to have a Cyclin E1 status above a predetermined threshold.

### INCORPORATION BY REFERENCE TO ANY PRIORITY APPLICATIONS

Any and all applications for which a foreign or domestic priority claim is identified in the Application Data Sheet as filed with the present application are hereby expressly incorporated by reference under 37 CFR 1.57, and Rules 4.18 and 20.6, including U.S. Provisional Application Nos. 63/382,817, filed Nov. 8, 2022, 63/485,764, filed Feb. 17, 2023, 63/459,520, filed Apr. 14, 2023, 63/504,166, filed May 24, 2023, 63/506,023, filed Jun. 2, 2023 and 63/588,235, filed Oct. 5, 2023, each of which are incorporated by reference in their entireties including any drawings. The present application is a continuation of PCT Application No. PCT/US2023/078809, filed Nov. 6, 2023, which claims priority to U.S. Provisional Application Nos. 63/382,817, filed Nov. 8, 2022, 63/485,764, filed Feb. 17, 2023, 63/459,520, filed Apr. 14, 2023, 63/504,166, filed May 24, 2023, 63/506,023, filed Jun. 2, 2023 and 63/588,235, filed Oct. 5, 2023, each of which are incorporated by reference in their entireties including any drawings.

### BACKGROUND

DNA damage is typically resolved by repair proteins that either re-connect, or re-synthesize damaged DNA. However, incorrect replacement of nucleotides into DNA can cause mutations and other genetic alterations, genetic disease, and loss of protein function. Improper DNA repair can lead to cell death, tumor progression, and cancer. Cell cycle checkpoints are important for proper DNA repair, ensuring that cells do not progress with cellular replication until their genomic integrity is restored. Cyclin E1 (encoded by the CCNE1 gene) is involved in cell cycle regulation by binding to Cyclin-dependent kinases (CDKs), including CDK2, thereby promoting cell cycle progression. WEE1 is a nuclear kinase involved in the G2-M cell-cycle checkpoint arrest for DNA repair before mitotic entry and overexpressed in a variety of cancers.

### SUMMARY

The present disclosure is based in part on the discovery that certain Cyclin E1 statuses, such as increased Cyclin E1 protein expression levels sensitize subjects having diseases involving DNA damage repair defects (or deficiency or alteration) (e.g., cancer) to treatment with WEE1 inhibitor, Azenosertib (also identified as ZN-c3) as a monotherapy or a combination therapy with at least one second chemotherapeutic agent, or a pharmaceutically acceptable salt thereof, and that Cyclin E1 biomarker levels (e.g., Cyclin E1 protein overexpression that may or may not be accompanied by CCNE1 gene amplification) can be used to select subjects for treatment using Azenosertib. Subjects selected based on a Cyclin E1 biomarker predetermined threshold have significantly improved responses (e.g., tumor growth inhibition and increased progression free survival (PFS)) when treated with Azenosertib, or a pharmaceutically acceptable salt thereof, as a monotherapy or a combination therapy with at least one second chemotherapeutic agent, or a pharmaceutically acceptable salt thereof.

Combination therapies with Azenosertib, or a pharmaceutically acceptable salt thereof, and a second chemotherapeutic agent, or a pharmaceutically acceptable salt thereof, further can provide a synergistic effect and improve subject outcomes. Accordingly, the present disclosure provides, among other things, methods of treating cancer with WEE1 inhibitor, Azenosertib (including pharmaceutically acceptable salts), alone or in combination with a second chemotherapeutic agent, or a pharmaceutically acceptable salt thereof, using a predetermined Cyclin E1 status, or a Cyclin E1 biomarker level above a predetermined threshold. In some embodiments, Cyclin E1 status(es) or Cyclin E1 biomarker level(s) are used as a predictive biomarker.

In a first aspect, the present disclosure provides a method of treating cancer comprising, administering to a subject selected to have a predetermined Cyclin E1 status, or a Cyclin E1 biomarker level above a predetermined threshold, an effective dose of Azenosertib, or a pharmaceutically acceptable salt thereof. In some embodiments, the predetermined cut-off or the predetermined threshold is a percentage of viable tumor cells having a Cyclin E1 immunohistochemistry (IHC) staining intensity of 2+ of above 30%. In some embodiments, the predetermined cut-off or the predetermined threshold is a Cyclin E1 IHC H-score of above 125.

In a second aspect, the present disclosure provides a method of treating cancer comprising, administering to a subject selected to have a predetermined Cyclin E1 status, or a Cyclin E1 biomarker level above a predetermined threshold, an effective dose of Azenosertib, or a pharmaceutically acceptable salt thereof, and a second chemotherapeutic agent, or a pharmaceutically acceptable salt thereof. In some embodiments, the predetermined threshold is a percentage of viable tumor cells having a Cyclin E1 IHC staining intensity of 2+ of above 10%. In some embodiments, the predetermined threshold is a percentage of viable tumor cells having a Cyclin E1 IHC staining intensity of 2+ of above 30%. In some embodiments, the predetermined cut-off or the predetermined threshold is a Cyclin E1 IHC H-score of above 50. In some embodiments, the predetermined cut-off or the predetermined threshold is a Cyclin E1 IHC H-score of above 125.

In a fourth aspect, the present disclosure provides a method of treating ovarian cancer comprising, administering to a subject selected to have a predetermined Cyclin E1 status, or a Cyclin E1 biomarker level above a predetermined threshold, an effective dose of Azenosertib, or a pharmaceutically acceptable salt thereof. In some embodiments, the predetermined threshold is a percentage of viable tumor cells having a Cyclin E1 IHC staining intensity of 2+ of above 30%. In some embodiments, the predetermined threshold is a Cyclin E1 IHC H-score of above 125.

In a fifth aspect, the present disclosure provides a method of treating ovarian cancer comprising, administering to a subject selected to have a predetermined Cyclin E1 status, or a Cyclin E1 biomarker level above a predetermined threshold, an effective dose of Azenosertib, or a pharmaceutically acceptable salt thereof, for a treatment cycle, and administering a second chemotherapeutic agent, or a pharmaceutically acceptable salt thereof, one or more times during the treatment cycle. In some embodiments, the predetermined threshold is a percentage of viable tumor cells having a Cyclin E1 IHC staining intensity of 2+ of above 10%. In some embodiments, the predetermined threshold is a percentage of viable tumor cells having a Cyclin E1 IHC staining intensity of 2+ of above 30%. In some embodiments, the predetermined threshold is a Cyclin E1 IHC H-score of above 50. In some embodiments, the predetermined threshold is a Cyclin E1 IHC H-score of above 125.

Various embodiments of the first, second, third, fourth, and fifth aspects of this disclosure as set forth above are described in the following paragraphs in the summary and also in the detailed description.

In some embodiments, the predetermined Cyclin E1 status is Cyclin E1-positive or Cyclin E1-high. In some embodiments, the predetermined Cyclin E1 status is Cyclin E1-positive (low) or Cyclin E1-positive (high). In one embodiment, the predetermined Cyclin E1 status is a Cyclin E1 protein expression level above a predetermined cut-off.

In some embodiments, the predetermined Cyclin E1 status or the Cyclin E1 biomarker level is measured by a Cyclin E1 protein expression level. In some embodiments, the Cyclin E1 protein expression level is determined by CCNE1 mRNA or transcript levels. In some embodiments, the Cyclin E1 protein expression level is determined by protein levels.

In some embodiments, the predetermined Cyclin E1 status or the Cyclin E1 biomarker level is an immunochemistry (IHC) status.

In one embodiment, the predetermined cut-off or the predetermined threshold is measured by a percentage of viable tumor cells having a Cyclin E1 immunohistochemistry (IHC) staining intensity of 2+.

In one embodiment, the predetermined cut-off or the predetermined threshold is a percentage of viable tumor cells having a Cyclin E1 IHC staining intensity of 2+ of above 7%, 8%, 9%, 10%, 11%, 12%, 13%, 14%, 15%, 16%, 17%, 18%, 19%, 20%, 21%, 22%, 23%, 24%, 25%, 26%, 27%, 28%, 28%, 29%, 30%, 31%, 32%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, or 62%.

In one embodiment, the predetermined cut-off or the predetermined threshold is a percentage of viable tumor cells having a Cyclin E1 IHC staining intensity of 2+ of above 10%. In one embodiment, the predetermined cut-off or the predetermined threshold is a percentage of viable tumor cells having a Cyclin E1 IHC staining intensity of 2+ of above 30%.

In some embodiments, the Cyclin E1 expression level is measured by a Cyclin E1 IHC H-score.

In some embodiments, the predetermined cut-off or the predetermined threshold for the Cyclin E1 IHC H-score is above 45, 50, 55, 60, 65, 70, 75, 80, 85, 90, 95, 100, 105, 110, 115, 120, 125, 130, 135, 140, 145, 150, 155, or 160.

In some embodiments, the predetermined cut-off or the predetermined threshold for the Cyclin E1 IHC H-score is above 50.

In some embodiments, the predetermined cut-off or the predetermined threshold for the Cyclin E1 IHC H-score is above 125.

In some embodiments, the predetermined Cyclin 1 status or the Cyclin E1 biomarker level is independent of CCNE1 gene amplification status in the subject. In an alternative embodiment, the predetermined Cyclin 1 status or the Cyclin E1 biomarker level is accompanied by a CCNE1 gene-amplified status in the subject.

In some embodiments, the CCNE1 gene amplification status is measured by a CCNE1 gene copy number.

In some embodiments, a CCNE1 gene-amplified status is a CCNE1 gene copy number of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

In some embodiments, a CCNE1 gene-amplified status is a CCNE1 gene copy number of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

In some embodiments, a CCNE1 gene-amplified status is a CCNE1 gene copy number of at least 6, 7, 8, 9, 10, 11, 12, 13, 14, 15, 16, 17, 18, 19, 20, 21, 22, 23, 24, 25, 26, 27, 28, 29, 30, 31, 32, 33, or 34.

In some embodiments, a CCNE1 gene-amplified status is a CCNE1 gene copy number of at least 3. In some embodiments, a CCNE1 gene-amplified status is a CCNE1 gene copy number of at least 4. In some embodiments, a CCNE1 gene-amplified status is a CCNE1 gene copy number of at least 5. In some embodiments, a CCNE1 gene-amplified status is a CCNE1 gene copy number of at least 6. In some embodiments, a CCNE1 gene-amplified status is a CCNE1 gene copy number of at least 7. In some embodiments, a CCNE1 gene-amplified status is a CCNE1 gene copy number of at least 8. In some embodiments, a CCNE1 gene-amplified status is a CCNE1 gene copy number of at least 9. In some embodiments, a CCNE1 gene-amplified status is a CCNE1 gene copy number of at least 10. In some embodiments, a CCNE1 gene-amplified status is a CCNE1 gene copy number of at least 11. In some embodiments, a CCNE1 gene-amplified status is a CCNE1 gene copy number of at least 12. In some embodiments, a CCNE1 gene-amplified status is a CCNE1 gene copy number of at least 14.

In some embodiments, a CCNE1 gene-amplified status is a CCNE1 gene copy number of at least 7.

In some embodiments, the subject is selected without determining level(s) and status(es) of other oncogenes.

In some embodiments, the other oncogenes are selected from BRCA1, BRCA2, TP53, PKMYT1, and PPP2R1A.

In some embodiments, the subject is selected without determining the levels of BRCA1 and/or BRCA2.

In some embodiments, the subject is selected without determining the levels of TP53.

In some embodiments, the cancer is a Cyclin E1-driven cancer.

In some embodiments, the cancer is selected from glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colon cancer, colorectal cancer, endometrial cancer, endometrium cancer, esophagus cancer, eye cancer, gallbladder cancer, gastric cancer, gastrointestinal cancer, Hodgkin lymphoma, Non-Hodgkin lymphoma, hematological tumor, head cancer, hematologic malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, uterine sarcoma, uterine serous carcinoma (USC), uterine CS, vaginal cancer, vulvar cancer, Waldenstrom macroglobulinemia, Wilms tumor, solid tumor, or liquid tumor, HGSOC, invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PC PGs), cholangiocarcinoma, acute myeloid leukemia (AML), CLL (Chronic lymphocytic leukemia), ALL (Acute Lymphocytic Leukemia), myelodysplastic syndromes (MDS), thymona, BRA F mutant metastatic colorectal cancer, uveal melanoma, high-grade serous ovarian, fallopian tube, or primary peritoneal cancer, BRAF V600E-mutated colorectal cancer, platinum-sensitive ovarian cancer, poly(ADP-ribose) polymerase inhibitor (PARPi)-resistant ovarian cancer, platinum-resistant ovarian cancer, platinum-refractory ovarian cancer, advanced pancreatic adenocarcinoma, pancreatic ductal adenocarcinoma, neuroendocrine tumor, neuroendocrine prostate cancer, pancreatic neuroendocrine tumor, small cell lung cancer (SCLC), germ cell cancer, and stromal cancer.

In some embodiments, the cancer is a cancer of an organ selected from adrenal gland, ampulla of vater, biliary tract, bladder/urinary tract, bone, bowel, breast, cervix, CNS/brain, esophagus/stomach, eye, head and neck, kidney, liver, lung, lymphoid, myeloid, ovary/fallopian tube, pancreas, penis, peripheral nervous system, peritoneum, pleura, prostate, skin, soft tissue, testis, thymus, thyroid, uterus, vulva/vagina, adenocarcinoma in situ, extra gonadal germ cell tumor (EGCT), a mixed cancer type, high-grade neuroendocrine carcinoma of the ovary, high-grade serous fallopian tube cancer (HGSFT), ovarian choriocarcinoma, and ovarian carcinoma NOS (OCNOS).

In some embodiments, the cancer is a solid tumor or a hematologic malignancy.

In some embodiments, the cancer is a solid tumor.

In some embodiments, the solid tumor is selected from endometrial cancer, gallbladder cancer, ovarian cancer (e.g., HGSOC), endometrium cancer, melanoma, colorectal cancer, bladder cancer, breast cancer (e.g., invasive, Triple Negative Breast Cancer (TNBC)), prostate cancer, Lung cancer (e.g., NSCLC, SCLC), esophagogastric cancer, gastric cancer, esophageal cancer, renal cancer (e.g., pRCC, ccRCC, chromophobe RCC), head and neck cancer, osteosarcoma cancer, pancreatic cancer, brain cancer, uterine CS, uterine cancer, adenoid cystic carcinoma (ACC), mesothelioma, cervical cancer, Diffuse large B cell lymphoma (DLBCL), non-Hodgkin lymphoma (NHL), liver cancer, glioblastoma (GBM), testicular cancer, Low-grade gliomas (LGGs), Pheochromocytoma and paraganglioma (PCPGs), cholangiocarcinoma, thyroid cancer, thymona, and uveal melanoma.

In some embodiments, the cancer is acute myeloid leukemia (AML).

In some embodiments, the tumor is selected from the group consisting of SCLC, neuroendocrine tumor, neuroendocrine prostate cancer, and pancreatic neuroendocrine tumor.

In some embodiments, the solid tumor is ovarian cancer.

In some embodiments, the ovarian cancer is epithelial ovarian cancer, germ cell cancer, or stromal cancer.

In some embodiments, the ovarian cancer is epithelial ovarian cancer,

In some embodiments, the ovarian cancer is high grade serous ovarian cancer (HGSOC). In some embodiments, the ovarian cancer is platinum-resistant ovarian cancer (PROC). In some embodiments, the ovarian cancer is PARP inhibitor-resistant. In some embodiments, the ovarian cancer is CCNE1 gene-amplified ovarian cancer. In some embodiments, the ovarian cancer is a Cyclin E1-overexpressing cancer. In some embodiments, the ovarian cancer is a Cyclin E1-overexpressing/non-CCNE1 gene-amplified cancer.

In some embodiments, the cancer is histologically and/or cytologically confirmed or the cancer is pathologically confirmed.

In some embodiments, the cancer is recurrent or persistent. In some embodiments, the cancer is metastatic. In some embodiments, the cancer is unresectable.

In some embodiments, the subject has received no more than 1, at least 1, 1, 2, 3, 4, 1 or 2, 1 to 2, 1 to 3, or 1 to 4 prior line(s) of therapy, prior line(s) of therapy in the advanced or metastatic setting, prior line(s) of chemotherapy, prior line(s) of platinum-based chemotherapy, prior regimen(s), or prior therapeutic regimen(s).

In some embodiments, the cancer is platinum-resistant, platinum-sensitive, or platinum-refractory.

In one embodiment, the method of treating comprises administering an effective dose of Azenosertib, or a pharmaceutically acceptable salt thereof, without in combination with a second chemotherapeutic agent, or a pharmaceutically acceptable salt thereof. In an alternative embodiment, the method of treating comprises administering an effective dose of Azenosertib, or a pharmaceutically acceptable salt thereof, in combination with a second chemotherapeutic agent, or a pharmaceutically acceptable salt thereof.

In some embodiments, the cancer is PARP inhibitor-resistant.

In some embodiments, the second chemotherapeutic agent is selected from carboplatin, cisplatin, paclitaxel, docetaxel, pegylated liposomal doxorubicin (PLD), doxorubicin, gemcitabine, cytarabine, fludarabine, fluorouracil (5-FU), irinotecan, topotecan, temozolomide, triapine, 5-azacytidine, capecitabine, AraC-FdUMP[10] (CF-10), cladribine, decitabine, hydroxyurea, oxaliplatin, niraparib, encorafenib, and cetuximab, or a pharmaceutically acceptable salt of any of the foregoing.

In some embodiments, the second chemotherapeutic agent is selected from azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate, and vincristine, or a pharmaceutically acceptable salt of any of the foregoing.

In some embodiments, the second chemotherapeutic agent is carboplatin, paclitaxel, gemcitabine, or pegylated liposomal doxorubicin (PLD), or a pharmaceutically acceptable salt of any of the foregoing. In some embodiments, the second chemotherapeutic agent is carboplatin, or a pharmaceutically acceptable salt thereof. In some embodiments, the second chemotherapeutic agent is paclitaxel, or a pharmaceutically acceptable salt thereof. In some embodiments, the second chemotherapeutic agent is gemcitabine, or a pharmaceutically acceptable salt thereof. In some embodiments, the second chemotherapeutic agent is pegylated liposomal doxorubicin (PLD), or a pharmaceutically acceptable salt thereof.

In some embodiments, the method comprises selecting a subject having the predetermined Cyclin E1 status, or the Cyclin E1 biomarker level above the predetermined threshold.

In some embodiments, Azenosertib, or a pharmaceutically acceptable salt thereof, and the second chemotherapeutic agent, or a pharmaceutically acceptable salt thereof, are administered concurrently.

In some embodiments, Azenosertib, or a pharmaceutically acceptable salt thereof, and the second chemotherapeutic agent, or a pharmaceutically acceptable salt thereof, are administered sequentially.

In some embodiments, Azenosertib, or a pharmaceutically acceptable salt thereof, and/or the second chemotherapeutic agent, or a pharmaceutically acceptable salt thereof, are administered intermittently.

In some embodiments, the method of treating comprises a step of selecting a subject having the predetermined Cyclin E1 status, or the Cyclin E1 biomarker level above the predetermined threshold.

In some embodiments, the method of treating comprises first determined the Cyclin E1 status or the Cyclin E1 biomarker level prior to the selecting step.

In some embodiments, the method of treating results in a subject overall response rate (ORR) at or greater than 10%, 15%, 20%, 25%, 30%, 35%, 40%, 45%, or 50%. In some embodiments, the overall response rate (ORR) is measured by complete response (CR), partial response (PR), CA-125 50% response, or a combination thereof.

In some embodiments, the method of treating results in a subject median progression-free survival (mPFS) of 5 months, 6 months, 7 months, 8 months, 9 months, 10 months, 11 months, 12 months or longer.

In a sixth aspect, the present disclosure provides a method of treating ovarian cancer comprising administering to a subject selected to have a predetermined Cyclin E1 status, or a Cyclin E1 biomarker level above a predetermined threshold, an effective dose of Azenosertib, or a pharmaceutically acceptable salt thereof, for a treatment cycle, and administering a second chemotherapeutic agent, or a pharmaceutically acceptable salt thereof, one or more times during the treatment cycle. All various embodiments described above for the first, second, third, fourth, and fifth aspects of the disclosure expressly apply to this sixth aspect.

In some embodiments, the treatment cycle is 21 days or 28 days.

In some embodiments, the treatment cycle is repeated.

Other features, objects, and advantages are apparent in the detailed description that follows. It should be understood, however, that the detailed description, while indicating embodiments, is given by way of illustration only, not limitation. Various changes and modifications within the scope of the present disclosure will become apparent to those skilled in the art from the detailed description.

### BRIEF DESCRIPTION OF THE DRAWINGS

Drawings are for illustration purposes only and not for limitation.

FIG. 1A-FIG. 1G show Cyclin E1 protein overexpression is associated with increased sensitivity to Azenosertib in ovarian cancer cell lines. FIG. 1A shows exemplary results demonstrating Azenosertib sensitivity correlates with Cyclin E1 protein expression in cancer cell lines, OV90, Kuramochi, TYK-nu and OVCAR3 assessed by CellTiter Glo after 96 hours of culture. FIG. 1B shows OV90 cells (low endogenous expression levels of Cyclin E1) transduced with a lentiviral vector expressing the CCNE1 gene increased sensitivity to Azenosertib. FIG. 1C shows Cyclin E1 protein expression by western blot of cell lines KURAMOCHI, COV362 and OV90 control (empty vector) and lentiviral induced Cyclin E1 protein overexpression. FIG. 1D shows growth rate inhibition and IC₅₀ in empty vector and Cyclin E1. FIG. 1E shows Cyclin E1 protein expression by western blot of Cyclin E1 in cell lines KURAMOCHI, COV362, JOM1, ES2, TYK-nu, CAVO3, OAW28, OVCAR4, OVCAR3 and OV90 control (empty vector). FIG. 1F shows a graph of Cyclin E1 protein expression level H-scores as a function of Cyclin E1 levels. FIG. 1G shows a graph of growth rate (GRₘₐₓ) value as a function of Cyclin E1 levels.

FIG. 2 shows exemplary growth rate inhibition (GR) of OV90, Kuramochi, OVCAR8, TYK-nu, Cov362, OVCAR3 and Caov3 cells in the presence of Azenosertib.

FIG. 3A shows downregulation of CDK2 was assessed by western blot, after treatment by siRNA. FIG. 3B shows a graph of percent viability as the concentration of Azenosertib increases. FIG. 3C shows a graph of growth rate inhibition as the concentration of Azenosertib increases.

FIGS. 4A-4D shows changes in replication stress markers in SKOV3 Cyclin E1-low cells after treatment with Azenosertib (80 mg/kg) and OVCAR3 Cyclin E1-high cells after treatment with Azenosertib (80 mg/kg). FIG. 4A shows baseline Cyclin E1 protein expression in SKOV3 Cyclin E1-low cells and OVCAR3 Cyclin E1-high cells, as examined by immunohistochemistry (IHC). FIG. 4B shows a graph depicting reduction in CDK1Y 15 level before and after treatment with Azenosertib. FIG. 4C shows the γH2AX (replication stress marker) level in SKOV3 Cyclin E1-low cells and OVCAR3 Cyclin E1-high cells. FIG. 4D shows γH2AX western blot in SKOV3 Cyclin E1-low cells and OVCAR3 Cyclin E1-high cells after treatment with Azenosertib.

FIGS. 5A-5D show exemplary reduction in tumor volume of SKOV3 (non-CCNE1-amplified, CN=2) CDX mice treated with Azenosertib compared to vehicle control (FIGS. 5A and 5C) and change in body weight (FIGS. 5B and 5D).

FIG. 6A and FIG. 6B show exemplary reduction in tumor volume of OVCAR8 (non-CCNE1-amplified) tumor induced mice treated with Azenosertib compared to vehicle control (FIG. 6A) and change in body weight (FIG. 6B).

FIG. 7A and FIG. 7B show exemplary reduction in tumor volume of HCC1806 (CCNE1 amplified, CN=7) CDX mice treated with Azenosertib compared to vehicle control (FIG. 7A) and change in body weight (FIG. 7B).

FIGS. 8A-8D show exemplary reduction in tumor volume of OVCAR3 (CCNE1 amplified, CN=14) CDX mice treated with Azenosertib compared to vehicle control (FIG. 8A and FIG. 8C) and change in body weight (FIG. 8B and FIG. 8D).

FIG. 9 shows exemplary synergy analysis of Azenosertib in combination with gemcitabine in OV90, OVCAR8, and OVCAR3 cells.

FIG. 10A and FIG. 10B show exemplary reduction in tumor volume of A 2780 (non-CCNE1-amplified, CN=2) tumor induced mice treated with Azenosertib alone or in combination with paclitaxel compared to vehicle control (FIG. 10A) and change in body weight (FIG. 10B).

FIG. 11A and FIG. 11B show exemplary reduction in tumor volume of OVCAR3 (CCNE1 amplified, CN=14) tumor induced mice treated with Azenosertib alone or in combination with paclitaxel compared to vehicle control (FIG. 11A) and change in body weight (FIG. 11B). FIG. 11C shows a heat map showing synergistic effect of chemotherapy (paclitaxel) and Azenosertib treatment in Cyclin E1^{high} cells (OVCAR3) as compared to Cyclin E1^{low} cells (OV90 and TY L-nu).

FIGS. 12A-12C show exemplary Cyclin E1 IHC H-score correlation with response in human subjects treated with Azenosertib in combination with carboplatin, paclitaxel, PLD, or Gemcitabine.

FIGS. 13A-13E show exemplary Cyclin E1 IHC H-score correlation with tumor response (FIG. 13A and FIG. 13C), progression free survival (FIG. 13B), CA 125 response (FIG. 13D) in human subjects. Subjects were grouped by low Cyclin E1 IHC H-score (<70), intermediate Cyclin E1 IHC H-score (70-130) and high Cyclin E1 IHC H-score (>130) (FIG. 13E).

FIGS. 14A-14C show CCNE1 gene amplification status and Cyclin E1 IHC H-score of subjects administered Azenosertib. FIG. 14D is an exemplary scatter plot demonstrating Cyclin E1 IHC H-score was highly correlated with CCNE1 transcript level. FIG. 14E is an exemplary image of tumor cells being designated with a Cyclin E1-positive status while FIG. 14F is an exemplary image of tumor cells being designated with a Cyclin E1-negative status. FIGS. 14G and 14H show exemplary Cyclin E1 IHC H-score correlation with response in human subjects treated with Azenosertib in combination with carboplatin, gemcitabine, paclitaxel, or PLD, and that subjects with either partial or complete clinical response (PR or CR, respectively) were associated with Cyclin E1 IHC protein expression levels and H-score of >50. FIGS. 14I and 14J show exemplary percentage of viable tumor cells having a Cyclin E1 IHC staining intensity of 2+ correlation with response in human subjects treated with Azenosertib in combination with carboplatin, gemcitabine, paclitaxel, or PLD, and that subjects with either partial or complete clinical response (PR or CR, respectively) were associated with Cyclin E1 IHC protein expression levels and a percentage of viable tumor cells of >10%. FIG. 14K is a graph mapping a percentage of viable tumor cells having a staining intensity of 2+ to an H-score cut-offs for Cyclin E1 immunohistochemistry (IHC).

FIG. 15A shows systemic therapy and responses prior to Azenosertib study enrollment. Subjects were classified as “Platinum Sensitive” or “Platinum Resistant” at the time of specimen collection. For each subject (y-axis), the timeline (x-axis) is centered at the time of specimen collection. Each treatment line (data segments) and events (data points) are indicated. Treatment class+responses and event types are coded by color (platinum=platinum) and shape respectively. CR: complete response, PR: partial response, SD: stable disease; PD: progressive disease; NE: not evaluable; and NA: not available.

FIG. 15B shows relative distribution of the Cyclin E1 protein expression status (i.e., Cyclin E1-negative, Cyclin E1-positive (low), Cyclin E1-positive (high)) (y-axis) across key subsets (x-axis) for clinical variables: platinum response, prior platinum exposure, HRR status and subject age at collection (panels). This data demonstrates that platinum exposure, response, or HRR mutational status did not significantly impact Cyclin E1 protein expression implying that Cyclin E1 positivity is frequent in High Grade Serous Ovarian Cancer and independent of prior platinum treatment.

FIGS. 16A and 16B are exemplary pathological scans of a subject with CCNE1-amplified Platinum Resistant Ovarian Cancer at screening (FIG. 16A) and after treatment with Azenosertib (FIG. 16B).

### DEFINITIONS

Unless defined otherwise, all technical and scientific terms used herein have the same meaning as is commonly understood by one of ordinary skill in the art. All patents, applications, published applications and other non-patent publications referenced herein are incorporated by reference in their entirety unless stated otherwise. In the event that there are a plurality of definitions for a term herein, those in this section prevail unless stated otherwise.

As used herein, the term “about” has its usual meaning as understood by those skilled in the art and thus indicates that a value includes the inherent variation of error for the method being employed to determine a value, or the variation that exists among multiple determinations.

As used herein, the terms “modify” or “alter”, or any forms thereof, mean to modify, alter, replace, delete, substitute, remove, vary, or transform.

As used herein, the terms “function” and “functional” have their usual meaning as understood by those skilled in the art and thus refer to a biological, enzymatic, or therapeutic function.

As used herein, the term “endogenous” has its usual meaning as understood by those skilled in the art and thus refers to the native, or wild type property of a gene, protein, or cell. In some embodiments, the endogenous gene is the wild type sequence of said gene. In some embodiment, the endogenous protein is the wild type sequence of said protein. In some embodiments, the endogenous protein function is the wild type function and activity level of said protein. In some embodiments, the endogenous cell is the wild type cell.

The term “mutation” has its usual meaning as understood by those skilled in the art and refers to an alteration of genetic sequence. In some embodiments, cells have multiple mutations. In some embodiments, mutations are in coding regions of the genome. Mutations can range in size from a single nucleotide, to a large segment of the chromosome that includes multiple genes. In some embodiments, at least one mutation is silent, having no significant impact on gene expression or function. In some embodiments, at least one mutation has an impact on gene expression or function, such as gene amplification, overexpression, or enhanced copy number. In some embodiments, at least one mutation is silent, having no significant impact on protein expression or function. In some embodiments, at least one mutation has a small impact on protein expression or function. In some embodiments, at least one mutation has a moderate impact on protein expression or function. In some embodiments, at least one mutation has a large impact on protein expression or function. In some embodiments, at least one mutation prevents protein expression or function. Non-limiting examples of mutations include insertions, deletions, truncations, substitutions, duplications, translocations, and inversions. In some embodiments, mutations are “somatic,” or occurring in body cells and are not inheritable. In some embodiments, a subset of somatic cells in an organism have at least one mutation that other somatic cells do not have. In some embodiments, mutations are “germline,” or occurring in germ cells and are inheritable.

As disclosed herein, mutations can be monitored through a variety of sequencing, expression, or functional assays. Non-limiting examples include DNA sequencing, RNA sequencing, DNA hybridization, protein sequencing, targeted genomic sequencing, whole exome sequencing, whole genome sequencing, ATAC-sequencing, Sanger sequencing, PCR, qPCR, RT-PCR, RT-qPCR, Next Generation Sequencing, protein truncation test, DNA microarrays, heteroduplex analysis, denaturing gradient gel electrophoresis, nucleotide sequencing, single strand conformational polymorphism, restriction enzyme digestion assay, fluorescence in situ hybridization (FISH), comparative genomic hybridization, restriction fragment length polymorphism, amplification refractory mutation system PCR, nested PCR, multiplex ligation-dependent probe amplification, single strand conformational polymorphism, and oligonucleotide ligation assay. Mutations can also be monitored through a variety of antibody-based methods using biological samples including, but are not limited to, Western blotting, fluorescence activated cell sorting, immunofluorescence, immunohistochemistry, immunocytochemistry, immunoprecipitation, enzyme-linked immunosorbent assay, radioimmunoassays, and electrochemiluminescence assays.

The term “cancer” is used herein in its usual biological sense and understood by those skilled in the art. Thus, it can include the cancer of any cell type, such as but not limited to glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, hematologic malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor, and/or liquid tumor.

As used herein, the term “tumor” has its usual meaning as understood by those skilled in the art and refers to an abnormal growth of cells or tissue. In some embodiments, the tumor is benign. In some embodiments, the tumor is malignant. A tumor becomes a cancer when it metastasizes, or spreads to other areas of the body. The term “solid tumor” as used herein has its usual meaning as understood by those skilled in the art and refers to an abnormal mass of tissue that does not contain liquid areas or cysts. Non-limiting examples of solid tumors include sarcomas, carcinomas, or lymphomas. Many cancer tissues can form solid tumors, such as but not limited to breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and/or ovarian cancer. The terms “cancer” and “tumor” may generally be used interchangeably unless the context clearly indicates that a more specific meaning is intended.

The term “cell” as used herein has its usual meaning as understood by those skilled in the art and can refer to any cell type. In some embodiments, said cells are mammalian cells. In some embodiments, said cells are human cells.

The terms “individual”, “subject”, or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea or pigs. In some embodiments, the subject can be human. In some embodiments, the subject can be a child and/or an infant. In other embodiments, the subject can be an adult.

The term “cancer treatment” as used herein has its usual meaning as understood by those skilled in the art and refers to a therapeutic modality (such as surgery and/or radiation) or an anti-cancer agent such as a small molecule, compound, protein, or other medicant that is used to treat, inhibit, or prevent cancer. Non-limiting examples of common classes of anti-cancer agents usable with any one or more of the alternatives described herein include alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators: immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-Jun-N-terminal kinase (JNK) inhibitors, mitogen-activated protein kinase (MAPK) inhibitors, MDM2 inhibitors, MEK inhibitors, ERK inhibitors, MMP inhibitors, mT or inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinon, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin. Examples of chemotherapeutic agents useful for cancer treatment include carboplatin, cisplatin, paclitaxel, docetaxel, pegylated liposomal doxorubicin, doxorubicin, gemcitabine, cytarabine, fludarabine, fluorouracil (5-FU), irinotecan, topotecan, temozolomide, triapine, 5-azacytidine, capecitabine, AraC-FdUMP[10] (CF-10), cladribine, decitabine, hydroxyurea and/or oxaliplatin, or a pharmaceutically acceptable salt of any of the foregoing. Other examples of chemotherapeutic agents useful for cancer treatment include azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and/or vincristine, or a pharmaceutically acceptable salt of any of the foregoing.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the compounds described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

W here a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

As used herein, the term “equivalent dose” refers to an effective amount as described above of the compound, e.g. Azenosertib in other salt forms.

The term “break” or “break days” refers to a time period when Azenosertib is not administered or days without dosing, days off therapy, or break days. For example, break refers to a period subsequent to a dosing cycle or an intervening period when Azenosertib dosing is paused between dosing weeks.

The terms “platinum-resistant” or “platinum-refractory” when referring to a cancer means to a cancer that responds at first to treatment with drugs that contain the metal platinum, but then comes back within a certain period. For example, ovarian cancer that comes back within 6 months after treatment is considered platinum-resistant. In one embodiment, a cancer is platinum-refractory when there is progression within 90 days of the last-administered dose of a platinum-based regimen in any line.

The term “Cyclin E1” refers to the protein that is involved in cell cycle regulation by binding to cyclin-dependent kinases, including CDK2. The protein Cyclin E1 is encoded by the “CCNE1” gene, which is an oncogene in many cancers. The term “Cyclin E1 status” refers to the expression level of the Cyclin E1 protein, which can be categorized as, for example, Cyclin E1-negative, Cyclin E1-low, Cyclin E1-positive, Cyclin E1-positive (low), Cyclin E1-positive (high), or Cyclin E1-high. Overexpression of Cyclin E1 refers to the overexpression of the protein, not the overexpression or amplification of the CCNE1 gene. Depending on the subject stratification, in one embodiment, overexpression of Cyclin E1 is indicated by a Cyclin E1 status of Cyclin E1-positive, Cyclin E1-positive (low), Cyclin E1-positive (high), and Cyclin E1-high, or by a Cyclin E1 status of Cyclin E1-high, or by a Cyclin E1 status of Cyclin E1-positive. In a similar manner, in one embodiment, lack of expression or overexpression of Cyclin E1 is indicated by a Cyclin E1 status of Cyclin E1-negative or Cyclin E1-low. In the clinical setting, Cyclin E1 protein expression levels can be determined by, for example, immunohistochemistry (IHC) or Western Blot, whereby a sample (e.g., subject tumor tissue, subject tumor cells) is contacted with an anti-Cyclin E1 antibody, stained, and is subject to histological evaluation by a board-certified pathologist, who scores the intensity of nuclear staining of the viable tumor cells in the sample as 0 (negative), 1+ (negative, weak, or low), 2+ (positive, weakly positive, equivocal, weak to moderate, or moderate), or 3+ (positive, strongly positive, or high). In some embodiments, percentages of viable tumor cells having 0, 1+, 2+, or 3+ staining intensity are obtained. In some embodiments, said percentages are used to obtain histological scores or H-scores. As used herein, the terms “H-score,” “IHC H-score,” and “Cyclin E1 IHC H-score” and their unabbreviated and/or plural forms are used interchangeably.

Various aspects are described in detail in the following sections. The use of sections is not meant to limit the present disclosure. Each section can apply to any aspect of present disclosure. In this application, the use of “or” means “and/or” unless stated otherwise.

### DETAILED DESCRIPTION

The present disclosure provides, among other things, methods for treating cancer in a subject selected to have a predetermined Cyclin E1 status, or a Cyclin E1 biomarker level above a predetermined threshold. In particular, the present disclosure provides methods for treating conditions characterized by excessive cellular proliferation, such as cancer, by administering to a subject Azenosertib, a WEE1 inhibitor. In some aspects, the present disclosure provides methods for treating conditions characterized by excessive cellular proliferation, such as cancer, by administering to a subject Azenosertib (a WEE1 inhibitor), or a pharmaceutically acceptable salt thereof, whether as a monotherapy or in combination with one or more chemotherapeutic agents at an effective dose. It also relates to treating cancer in a subject identifying subjects having a Cyclin E1 biomarker level above a predetermined threshold without determining the levels of other cancer biomarkers in the subject.

The present disclosure further provides methods of treating ovarian cancer comprising administering to a subject selected to have a predetermined Cyclin E1 status, or a Cyclin E1 biomarker level above a predetermined threshold, an effective dose of Azenosertib, or a pharmaceutically acceptable salt thereof. The present disclosure further provides methods of treating ovarian cancer comprising administering to a subject selected to have a predetermined Cyclin E1 status, or a Cyclin E1 biomarker level above a predetermined threshold, an effective dose of Azenosertib, or a pharmaceutically acceptable salt thereof, for a treatment cycle, and administering a second chemotherapeutic agent, or a pharmaceutically acceptable salt thereof, one or more times during the treatment cycle.

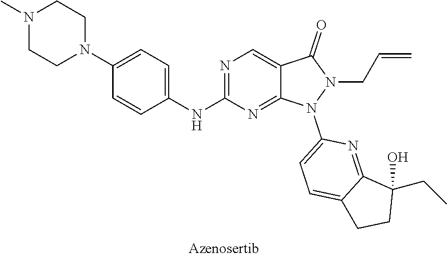 [SMILES: C=CCn1c(=O)c2cnc(Nc3ccc(N4CCN(C)CC4)cc3)nc2n1-c1ccc2c(n1)[C@@](O)(CC)CC2]

The compound Azenosertib and pharmaceutically acceptable salts thereof are WEE1 inhibitors. The chemical structure of the compound Azenosertib is depicted above. The compound Azenosertib and pharmaceutically acceptable salts thereof can be prepared in various ways. See, e.g., WO 2019/173082. WO 2019/173082 and WO 2021/231653 describe the compound Azenosertib and methods of using it to treat cancer.

Cyclin E1 (encoded by the CCNE1 gene) is involved in cell cycle regulation by binding to Cyclin-dependent kinases (CDKs), including CDK2, thereby promoting cell cycle progression. Cyclin E1 and cyclin E2 are encoded by CCNE1 gene at 19q12, and CCNE2 gene at 8q22.1, respectively. Cyclin E1 has crucial roles in cell proliferation and oncogenesis and Cyclin E2 is largely regarded as functionally redundant with cyclin E1. Cyclin E accumulates at the G1-S phase boundary and is degraded as cells progress through S phase. Cyclin E has multiple functions in cell cycle progression, both CDK2-dependent and CDK2-independent. Cyclin E/CDK2 complex controls G1/S phase transition and S phase progression by phosphorylating numerous proteins, regulates the apoptotic response to DNA damage via FOX 01 phosphorylation and plays a role in epigenetic regulation via EZH2 phosphorylation.

WEE1 is a tyrosine kinase that is a critical component of the ATR-mediated G2 cell cycle checkpoint control that prevents entry into mitosis in response to cellular DNA damage. WEE1 activation can lead to the selective phosphorylation of CDK2, thereby regulating CDK2-cyclin A/E complexes which control the G1/S phase progression. Inhibition of WEE1 can result in excessive replication activity, thereby leading to replication catastrophe. WEE1 inhibition has the potential to sensitize tumors to induce tumor cell death.

### Subject History and Selection

The CCNE1 gene is overexpressed and/or amplified in various cancers. Altered CCNE1 levels (e.g., gene amplification and/or gene overexpression) dysregulate cell cycle progression making cells more vulnerable to WEE1 inhibition. As CCNE1 levels increase, sensitivity to WEE1 inhibitors increase leading to improved efficacy in treating cancer with WEE1 inhibitors (e.g., Azenosertib).

Accordingly, the methods described herein use CCNE1 gene amplification status, predetermined Cyclin E1 status, and/or Cyclin E1 biomarker levels for selecting subjects for treating cancer with Azenosertib, or a pharmaceutically acceptable salt thereof. In some embodiments, the methods described herein comprise a step of selecting a subject having a predetermined Cyclin E1 status, or a Cyclin E1 biomarker level above the predetermined threshold.

In some embodiments, the method further comprises first determining the Cyclin E1 biomarker level prior to the selecting step.

In some embodiments, the subject has received one or more prior lines of therapy. In some embodiments, the subject has received 2 prior lines of therapy. In some embodiments, the subject has received 3 prior lines of therapy. In some embodiments, the subject has received 3 or more prior lines of therapy.

In some embodiments, the subject has a cancer that is relapsed or refractory. In some embodiments, the cancer is platinum-resistant. In some embodiments, the cancer is platinum-refractory. In some embodiments, the cancer is PARP inhibitor-resistant.

### Predetermined Cyclin E1 Statuses and Predetermined Cut-Offs Cyclin E1 Biomarker Levels and Predetermined Thresholds

The Cyclin E1 status and its predetermined cut-offs and the Cyclin E1 biomarker predetermined threshold can be determined by a variety of methods. In some embodiments, the predetermined threshold is an absolute value or standard. In some embodiments, the predetermined threshold is obtained from literature sources. In some embodiments, the predetermined threshold is obtained from the subject's own historical Cyclin E1 statuses Cyclin E1 biomarker levels. In some embodiments, the predetermined threshold is obtained from Cyclin E1 statuses and Cyclin E1 biomarker levels in a subject without cancer.

In some embodiments, the predetermined threshold is expressed by comparison to a reference or control. In some embodiments, the reference or control is tested and/or determined substantially simultaneously with the testing Cyclin E1 biomarker levels in the subject. In embodiments, the reference or control is a historical reference or control. In some embodiments, the reference or control may be based on the subject's Cyclin E1 levels prior to treatment with Azenosertib, or a pharmaceutically acceptable salt thereof.

### Cyclin E1 Overexpression

In some embodiments, the predetermined Cyclin E1 status, or the Cyclin E1 biomarker predetermined threshold is measured by a Cyclin E1 protein expression level.

In some embodiments, the Cyclin E1 protein expression level is determined by detecting the amount of CCNE1 mRNA (transcript) or Cyclin E1 protein. In some embodiments, the Cyclin E1 protein overexpression level is determined by mRNA or transcript levels. In some embodiments, the Cyclin E1 protein expression level is determined by protein levels. In some embodiments, the Cyclin E1 protein expression level is a Cyclin E1 protein expression level above a predetermined cut-off. In one embodiment, the predetermined cut-off or the predetermined threshold I is measured by a percentage of viable tumor cells having a Cyclin E1 immunohistochemistry (IHC) staining intensity of 2+. In some embodiments, the Cyclin E1 protein expression level is measured by a Cyclin E1 IHC H-score.

CCNE1 mRNA levels or Cyclin E1 protein expression levels can be measured any methods known in the art including but not limited to reporter gene, Northern blot, Western blot, Fluorescent in situ hybridization (FISH), Reverse transcription PCR, or RNA-Seq based assays.

In some embodiments, Cyclin E1 protein expression is determined using RNA detection of CCNE1 mRNA or transcript. In some embodiments, Cyclin E1 protein expression is determined using an RNA sequencing method.

In some embodiments, Cyclin E1 protein expression is measured by a quantitative readout. In some embodiments, Cyclin E1 protein expression is measured by a qualitative readout.

In some embodiments, Cyclin E1 protein expression is measured by signal intensity. In some embodiments, signal intensity is determined using a Western blot. In some embodiments, relative signal intensity is quantified.

### IHC Percentage of Viable Tumor Cells Having a Staining Intensity of 0, 1+, 2+, or 3+, and H-Scores

In some embodiments, Cyclin E1 protein expression level is measured by a percentage of viable tumor cells having a certain Cyclin E1 immunohistochemistry (IHC) staining intensity, i.e., an IHC staining intensity of 0, 1+, 2+, or 3+. In some embodiments, Cyclin E1 protein expression level is measured by a Cyclin E1 IHC H-score. In some embodiments, Cyclin E1 protein expression level is measured by a percentage of viable tumor cells having a certain Cyclin E1 IHC staining intensity in combination with a Cyclin E1 IHC H-score In some embodiments, H-scores are calculated using the method described in Diar Aziz, et. al., Gynecologic Oncology 151, 327-336 (2018). Briefly, an H-score is a semi-quantitative measurement derived by immunohistochemical staining of a subject's tumor cells. A subject's tumor cells are stained with Cyclin E1 antibodies using the Vetana Bench Mark ULTRA™ automated staining platform and the Optiview™ Detection Kit. The expression of each protein is assessed using a 0, 1+, 2+, or 3+ staining intensity by a trained and qualified observer with categorization of ambiguous cases confirmed by a pathologist and, in the case of Cyclin E1, the percentage of tumor cells having a Cyclin E1 IHC staining intensity (SI) of 0, 1+, 2+, or 3+ is obtained and used to determine Cyclin E1 protein expression level. Cyclin E1 is assessed based on nuclear staining while URI1 expression is assessed based on cytoplasmic staining.

A subject's H-score is determined by adding 3× the percentage of strongly staining cells (SI=3+)+2× the percentage of moderately staining cells (SI=2+)+1× the percentage of weekly staining cells (SI=1+). The H-score of a subject thus can range from 0 to 300. The formula to calculate the histological score (H-score) is <score=a*f1+b*f2+c*f3>, where fi is the fraction of cells at staining intensity of i, and i=1 (for 1+), 2 (for 2+) or 3 (for 3+). In some embodiments a=1, b=2, c=3. In some embodiments a=0, b=1, c=1 and the score is the fraction of cells at a staining intensity of 2+. In some embodiments a=0, b=0, c=1 and the score is the fraction of cells at a staining intensity of 3+.

In some embodiments, the Cyclin E1 status predetermined cut-off and the Cyclin E1 biomarker level predetermined threshold is measured by a percentage of viable tumor cells having a Cyclin E1 immunohistochemistry (IHC) staining intensity of 2+. In some embodiments, the predetermined cut-off or the predetermined threshold is a percentage of viable tumor cells having a Cyclin E1 IHC staining intensity of 2+ of above 7%, 8%, 9%, 10%, 11%, 12%, 13%, 14%, 15%, 16%, 17%, 18%, 19%, 20%, 21%, 22%, 23%, 24%, 25%, 26%, 27%, 28%, 28%, 29%, 30%, 31%, 32%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, or 62%. In some embodiments, the predetermined cut-off or the predetermined threshold is a percentage of viable tumor cells having a Cyclin E1 IHC staining intensity of 2+ of above 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 28%, 29%, 30%, 31%, 32%, 32%, 33%, 34%, 35%, 36%, 37%, 38%, 39%, or 40%.

In some embodiments, the predetermined cut-off or the predetermined threshold is a percentage of viable tumor cells having a Cyclin E1 IHC staining intensity of 2+ of above 10%. In some embodiments, the predetermined cut-off or the predetermined threshold is a percentage of viable tumor cells having a Cyclin E1 IHC staining intensity of 2+ of above 8-12%, or above 8-11%, or above 8-10%, or above 8-9%, or above 9-12%, or above 9-11%, or above 9-10%, or above 10-12%, or above 10-11%, or above 11-12%.

In some embodiments, the predetermined cut-off or the predetermined threshold is a percentage of viable tumor cells having a Cyclin E1 IHC staining intensity of 2+ of above 15%. In some embodiments, the predetermined cut-off or the predetermined threshold is a percentage of viable tumor cells having a Cyclin E1 IHC staining intensity of 2+ of above 13-17%, or above 13-16%, or above 13-15%, or above 13-14%, or above 14-16%, or above 14-16%, or above 14-15%, or above 15-17%, or above 15-16%, or above 16-17%.

In some embodiments, the predetermined cut-off or the predetermined threshold is a percentage of viable tumor cells having a Cyclin E1 IHC staining intensity of 2+ of above 20%. In some embodiments, the predetermined cut-off or the predetermined threshold is a percentage of viable tumor cells having a Cyclin E1 IHC staining intensity of 2+ of above 18-22%, or above 18-21%, or above 18-20%, or above 18-19%, or above 19-22%, or above 19-21%, or above 19-20%, or above 20-22%, or above 20-21%, or above 21-22%.

In some embodiments, the predetermined cut-off or the predetermined threshold is a percentage of viable tumor cells having a Cyclin E1 IHC staining intensity of 2+ of above 30%. In some embodiments, the predetermined cut-off or the predetermined threshold is a percentage of viable tumor cells having a Cyclin E1 IHC staining intensity of 2+ of above 28-32%, or above 28-31%, or above 28-30%, or above 28-29%, or above 29-32%, or above 29-31%, or above 29-30%, or above 30-32%, or above 30-31%, or above 31-32%.

In some embodiments, the predetermined cut-off or the predetermined threshold is a percentage of viable tumor cells having a Cyclin E1 IHC staining intensity of 2+ of above 35%. In some embodiments, the predetermined cut-off or the predetermined threshold is a percentage of viable tumor cells having a Cyclin E1 IHC staining intensity of 2+ of above 33-37%, or above 33-36%, or above 33-35%, or above 33-34%, or above 34-36%, or above 34-36%, or above 34-35%, or above 35-37%, or above 35-36%, or above 36-37%.

In some embodiments, the predetermined cut-off or the predetermined threshold is a percentage of viable tumor cells having a Cyclin E1 IHC staining intensity of 2+ of above 40%. In some embodiments, the predetermined cut-off or the predetermined threshold is a percentage of viable tumor cells having a Cyclin E1 IHC staining intensity of 2+ of above 38-42%, or above 38-41%, or above 38-40%, or above 38-39%, or above 39-42%, or above 39-41%, or above 39-40%, or above 40-42%, or above 40-41%, or above 41-42%.

In some embodiments, the predetermined cut-off or the predetermined threshold is a percentage of viable tumor cells having a Cyclin E1 IHC staining intensity of 2+ of above 45%. In some embodiments, the predetermined cut-off or the predetermined threshold is a percentage of viable tumor cells having a Cyclin E1 IHC staining intensity of 2+ of above 43-47%, or above 43-46%, or above 43-45%, or above 43-44%, or above 44-46%, or above 44-46%, or above 44-35%, or above 45-47%, or above 45-46%, or above 46-47%.

In some embodiments, the predetermined cut-off or the predetermined threshold is a percentage of viable tumor cells having a Cyclin E1 IHC staining intensity of 2+ of above 50%. In some embodiments, the predetermined cut-off or the predetermined threshold is a percentage of viable tumor cells having a Cyclin E1 IHC staining intensity of 2+ of above 48-52%, or above 48-51%, or above 48-50%, or above 48-49%, or above 49-52%, or above 49-51%, or above 49-50%, or above 50-52%, or above 50-51%, or above 51-52%.

In some embodiments, the predetermined cut-off or the predetermined threshold is a percentage of viable tumor cells having a Cyclin E1 IHC staining intensity of 2+ of above 55%. In some embodiments, the predetermined cut-off or the predetermined threshold is a percentage of viable tumor cells having a Cyclin E1 IHC staining intensity of 2+ of above 53-57%, or above 53-56%, or above 53-55%, or above 53-54%, or above 54-56%, or above 54-56%, or above 54-55%, or above 55-57%, or above 55-56%, or above 56-57%.

In some embodiments, the predetermined cut-off or the predetermined threshold is a percentage of viable tumor cells having a Cyclin E1 IHC staining intensity of 2+ of above 60%. In some embodiments, the predetermined cut-off or the predetermined threshold is a percentage of viable tumor cells having a Cyclin E1 IHC staining intensity of 2+ of above 58-62%, or above 58-61%, or above 58-60%, or above 58-69%, or above 59-62%, or above 59-61%, or above 59-60%, or above 60-62%, or above 60-61%, or above 61-62%.

In some embodiments, the Cyclin E1 status predetermined cut-off and the Cyclin E1 biomarker level predetermined threshold is measured by a percentage of viable tumor cells having a Cyclin E1 immunohistochemistry (IHC) staining intensity of 3+.

In some embodiments, the predetermined cut-off or the predetermined threshold is a Cyclin E1 IHC H-score of above 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300. In some embodiments, the threshold for the Cyclin E1 IHC H-score is above 40, 50, 60, 65, 70, 75, 80, 90, 95, 130 or 180.

In some embodiments, the predetermined cut-off or the predetermined threshold is a Cyclin E1 IHC H-score of above 45, 50, 55, 60, 65, 70, 75, 80, 85, 90, 95, 100, 105, 110, 115, 120, 125, 130, 135, 140, 145, 150, 155, or 160.

In some embodiments, the predetermined cut-off or the predetermined threshold is a Cyclin E1 IHC H-score of above 40. In some embodiments, the predetermined cut-off or the predetermined threshold is a Cyclin E1 IHC H-score of above 50. In some embodiments, the threshold for the predetermined cut-off or the predetermined threshold is a Cyclin E1 IHC H-score of above 60. In some embodiments, the predetermined cut-off or the predetermined threshold is a Cyclin E1 IHC H-score of above 65. In some embodiments, the predetermined cut-off or the predetermined threshold is a Cyclin E1 IHC H-score of above 70. In some embodiments, the predetermined cut-off or the predetermined threshold is a Cyclin E1 IHC H-score of above 75. In some embodiments, the threshold for the Cyclin E1 IHC H-score is above 80. In some embodiments, the predetermined cut-off or the predetermined threshold is a Cyclin E1 IHC H-score of above 90. In some embodiments, the predetermined cut-off or the predetermined threshold is a Cyclin E1 IHC H-score of above 95. In some embodiments, the threshold for the Cyclin E1 IHC H-score is above 125. In some embodiments, the predetermined cut-off or the predetermined threshold is a Cyclin E1 IHC H-score of above 130. In some embodiments, the predetermined cut-off or the predetermined threshold is a Cyclin E1 IHC H-score of above 135. In some embodiments, the threshold for the Cyclin E1 IHC H-score is above 150.

In some embodiments, the predetermined cut-off or the predetermined threshold is a Cyclin E1 IHC H-score of above 25. In some embodiments, the predetermined cut-off or the predetermined threshold is a Cyclin E1 IHC H-score of above 70. In some embodiments, the predetermined cut-off or the predetermined threshold is a Cyclin E1 IHC H-score of above 130.

In some embodiments, the percentage of viable tumor cells having a Cyclin E1 IHC staining intensity of 2+ predetermined cut-off or predetermined threshold and/or the Cyclin E1 IHC H-score predetermined cut-off or predetermined threshold is a predictive biomarker for treating a subject with a WEE1 inhibitor, such as Azenosertib (including pharmaceutically acceptable salts thereof), whether as a monotherapy, or in combination with one or more second chemotherapeutic agents, or a pharmaceutically acceptable salt thereof, e.g., to predict the subject's sensitivity, responsiveness to the treatment, including predicting the subject's tumor response, overall response rate (ORR) and/or median progression free survival (mPFS).

It is to be understood that, in both the Cyclin E1 IHC H-score and the percentage of viable tumor cells having a Cyclin E1 IHC staining intensity of 2+ scoring systems, The comparison to the score threshold or cut-off, X, to determine a positive status (e.g., Cyclin E1-positive) vs a negative status (e.g., Cyclin E1-negative) is generally expressed using strict inequality. Since scores are typically rounded to the nearest integer, calling positivity using a score strictly greater than X is equivalent to calling positivity using as a score greater or equal to X+1.

### CCNE1 Gene Amplification

CCNE1 gene amplification is the differential increase in the CCNE1 portion of the genome in relative to with the genome as a whole. In some embodiments, “gene amplification” or “increased CCNE1 gene amplification level” refers to any increase in gene copies relative to endogenous copies.

CCNE1 gene amplification level can be determined by methods known in the art. In some embodiments, CCNE1 gene amplification level is determined using an in situ hybridization (ISH) assay. In some embodiments, CCNE1 gene amplification level is determined using amplification by fluorescence in situ hybridization (FISH). In some embodiments, CCNE1 gene amplification level is determined using a quantitative polymerase chain reaction method. In some embodiments, CCNE1 gene amplification level is determined using next generation sequencing method.

### Copy Number

In some embodiments, the CCNE1 gene amplification level or a subject's CCNE1 gene amplification status is measured by CCNE1 gene copy number. In some embodiments, CCNE1 gene copy number is determined using the ISH assay described in Aziz et al., supra. Briefly, a pre-diluted ready to use 19q12 DNP ISH probe covering the coding sequence of CCNE1 and URI1 is used to measure CCNE1 gene amplification in conjuncture with an ISNR DIG ISH probe which serves as a surrogate reference for diploid copy number located at 19p13.2 in an ISH assay of the subject's tumor cells optimized on the Vetana ULTRA™ platform. Copy number can be determined when there is interpretable black (19q12) and red (INSR) signals in normal and malignant cells with at least 50 malignant cells and minimal background staining and is the average number of interpretable black signals per cell. In some embodiments, CCNE1 gene copy number is determined using whole genome or whole exsome sequencing method.

In some embodiments, a CCNE1 gene-amplified status is a CCNE1 gene copy number of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In some embodiments, a CCNE1 gene-amplified status is a CCNE1 gene copy number of is at least 7. In some embodiments a CCNE1 gene-amplified status is a CCNE1 gene copy number of at least 8. In some embodiments, a CCNE1 gene-amplified status is a CCNE1 gene copy number of at least 14.

In some embodiments, a CCNE1 gene-amplified status is a CCNE1 gene copy number of at least 3. In some embodiments, a CCNE1 gene-amplified status is a CCNE1 gene copy number of at least 4. In some embodiments, a CCNE1 gene-amplified status is a CCNE1 gene copy number of at least 5. In some embodiments, a CCNE1 gene-amplified status is a CCNE1 gene copy number of at least 6. In some embodiments, a CCNE1 gene-amplified status is a CCNE1 gene copy number of at least 7. In some embodiments, a CCNE1 gene-amplified status is a CCNE1 gene copy number of at least 8. In some embodiments, a CCNE1 gene-amplified status is a CCNE1 gene copy number of at least 9. In some embodiments, a CCNE1 gene-amplified status is a CCNE1 gene copy number of at least 10. In some embodiments, a CCNE1 gene-amplified status is a CCNE1 gene copy number of at least 11. In some embodiments, a CCNE1 gene-amplified status is a CCNE1 gene copy number of at least 12. In some embodiments, a CCNE1 gene-amplified status is a CCNE1 gene copy number of at least 13. In some embodiments, a CCNE1 gene-amplified status is a CCNE1 gene copy number of at least 14.

In some embodiments, a CCNE1 copy number of 2 (CN=2) is non-amplified. In some embodiments, a CCNE1 copy number between 2 and 5 (CN=2-5) is copy number gain. In some embodiments, a CCNE1 copy number of greater than 5 (CN>5) is amplified.

In some embodiments, a CCNE1 gene-amplified status based on a CCNE1 gene copy number cut-off is a predictive biomarker. In some embodiments, the CCNE1 gene copy number cut-off is a predictive biomarker for treating cancer with Azenosertib, or a pharmaceutically acceptable salt thereof. In some embodiments, the CCNE1 gene copy number cut-off is selected based on progression free survival. In some embodiments, the CCNE1 gene copy number cut-off is selected based on tumor response. In some embodiments, the CCNE1 gene copy number cut-off is selected based on clinical benefit rate (CBR). In some embodiments, the CCNE1 gene copy number cut-off is selected based on disease control rate (DCR). In some embodiments, the CCNE1 gene copy number cut-off is selected based on overall survival (OS).

### Additional Biomarkers

In some embodiments, subjects are selected based on a Cyclin E1 predetermined status or Cyclin E1 biomarker levels and levels of one or more additional biomarkers. In some embodiments, subjects have already been identified as having one or more additional biomarkers. In some embodiments, additional biomarkers are included in the selection criteria. In some embodiments, additional biomarkers are not included in the selection criteria.

In some embodiments, the subject is selected without determining levels of other cancer biomarkers. In some embodiments, subjects are selected based on Cyclin E1 biomarker levels only. In some embodiments, the subject is selected without determining levels of BRCA1 and/or BRCA2. In some embodiments, the subject is selected without determining the levels of TP53. In some embodiments, the subject is selected without determining the levels of CA 125.

In other embodiments, the subject is selected without determining the levels of other cancer biomarkers. In some embodiments, the subject is selected to have a predetermined level of a cancer biomarker other than Cyclin E1. In some embodiments, the subject is selected to have a BCRA1 and/or BRCA2 biomarker level below a predetermined threshold. In some embodiments, the subject is selected to have a TP53 biomarker level below a predetermined cut-off or threshold. In some embodiments, the subject is selected to have a CA 125 biomarker level below a predetermined cut-off threshold. In some embodiments, the subject is selected to have a BCRA1 and/or BRCA2 biomarker level above a predetermined cut-off or threshold. In some embodiments, the subject is selected to have a TP53 biomarker level above a predetermined cut-off or threshold. In some embodiments, the subject is selected to have a CA 125 biomarker level above a predetermined cut-off or threshold.

### Methods of Treatment

The present disclosure provides methods of treating cancer using the compound known as Azenosertib, or a pharmaceutically acceptable salt thereof, wherein subjects are selected to have a predetermined Cyclin E1 status, or a Cyclin E1 biomarker level above a predetermined threshold. Azenosertib is a WEE1 inhibitor of the formula:

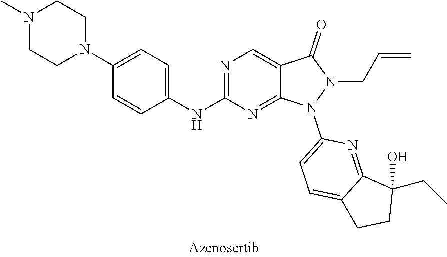 [SMILES: C=CCn1c(=O)c2cnc(Nc3ccc(N4CCN(C)CC4)cc3)nc2n1-c1ccc2c(n1)[C@@](O)(CC)CC2]

WO 2019/173082 and WO 2021/231653 describe the compound Azenosertib, both of which are hereby incorporated by reference in their entirety. Azenosertib is also known as ZN-c3 and these terms are used interchangeably.

In embodiments, a method described herein results in a therapeutic effect (e.g., a desired pharmacologic and/or physiologic effect). A therapeutic effect can encompass partially or completely curing a disease, relieving one or more adverse symptoms attributable to the disease, and/or delaying progression of the disease. To this end, the inventive method comprises administering a therapeutically effective amount of a therapeutic agent (e.g., Azenosertib, or a pharmaceutically acceptable salt thereof, and/or a second chemotherapeutic agent, or a pharmaceutically acceptable salt thereof). A therapeutically effective amount can be an amount effective, at dosages and for periods of time necessary, to achieve a desired therapeutic result (e.g., tumor growth inhibition, progression free survival, complete response, partial response etc.). A therapeutically effective amount may vary according to factors such as the disease state, age, sex, and weight of the individual, and the ability of the binding agent to elicit a desired response in the individual.

### Administration Routes

In some embodiments, the effective dose of Azenosertib, or a pharmaceutically acceptable salt thereof, is administered orally, intravenously, or subcutaneously. In some embodiments, the effective dose of Azenosertib, or a pharmaceutically acceptable salt thereof, is administered orally. One may also use alternative suitable techniques of administering the effective dose of Azenosertib, or a pharmaceutically acceptable salt thereof, that are known to those skilled in the art including, but not limited to, oral, rectal, pulmonary topical, aerosol, injection, infusion and parenteral delivery, including intramuscular, subcutaneous, intravenous, intramedullary injections, intrathecal, direct intraventricular, intraperitoneal, intranasal and intraocular injections. In other embodiments, Azenosertib, or a pharmaceutically acceptable salt thereof, and/or chemotherapeutic, can be administered orally.

In some embodiments, the effective dose of Azenosertib, or a pharmaceutically acceptable salt thereof, is administered orally, intravenously, subcutaneously, intrathecally, intramuscularly, intracavitary, intrapleural, intralesional, or intra-arterial. In some embodiments, the effective dose of Azenosertib, or a pharmaceutically acceptable salt thereof, is administered orally, intravenously, or subcutaneously. In some embodiments, the effective dose of Azenosertib, or a pharmaceutically acceptable salt thereof, is administered intrathecally, intramuscularly, intracavitary, intrapleural, intralesional, or intra-arterial.

In some embodiments, the effective dose of Azenosertib, or a pharmaceutically acceptable salt thereof, is administered orally.

### Azenosertib Dosage and Schedule

In some embodiments, methods described herein comprises intermittent dosing, i.e. comprises consecutive days of dosing followed by break days, in one or more dosing cycles comprising intervening break weeks. In some embodiments, methods described herein comprise continuous dosing. In some embodiments, methods described herein comprise combination therapy comprises continuous dosing of one of the agents. In some embodiments, methods described herein comprise combination therapy comprises continuous dosing of one of the agents and intermittent dosing of Azenosertib, or a pharmaceutically acceptable salt thereof.

In some embodiments, the Azenosertib, or a pharmaceutically acceptable salt thereof, is administered based on body weight of the subject. In some embodiments, the effective dose of Azenosertib, or a pharmaceutically acceptable salt thereof, is between 2 mg/kg and 20 mg/kg. In some embodiments, the effective dose Azenosertib, or a pharmaceutically acceptable salt thereof, is between 2-18 mg/kg, 2-16 mg/kg, 2-14 mg/kg, 2-12 mg/kg, 2-10 mg/kg, 2-8 mg/kg, 2-6 mg/kg, 3-4 mg/kg, 3-5 mg/kg, or 4-6 mg/kg. In some embodiments, the effective dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, at least 15 mg/kg, at least 16 mg/kg, at least 17 mg/kg, at least 18 mg/kg, or at least 19 mg/kg.

In some embodiments, Azenosertib may also be in the form of equivalent dose (e.g., the compound in other salt forms). In some embodiments, the effective dose is a flat dose. In some embodiments, the effective dose of Azenosertib, or a pharmaceutically acceptable salt thereof, ranges from 200-800 mg, or equivalents thereof, once a day. In some embodiments, the effective dose of Azenosertib, or a pharmaceutically acceptable salt thereof, ranges from 200-600 mg, or equivalents thereof, once a day. In some embodiments, the effective dose of Azenosertib, or a pharmaceutically acceptable salt thereof, ranges from 300-600 mg, or equivalents thereof, once a day. In some embodiments, the effective dose of Azenosertib, or a pharmaceutically acceptable salt thereof, ranges from 400-600 mg, or equivalents thereof, once a day. In some embodiments, the effective dose of Azenosertib, or a pharmaceutically acceptable salt thereof, ranges from 400-800 mg, or equivalents thereof, once a day. In some embodiments, the effective dose of Azenosertib, or a pharmaceutically acceptable salt thereof, ranges from 50-350 mg, 50-290 mg, 100-290 mg, 100-250 mg, 150-250 mg, or 180-220 mg once a day, or equivalents thereof. In some embodiments, the effective dose of Azenosertib, or a pharmaceutically acceptable salt thereof, ranges from 50-400 mg, 100-400 mg, 150-400 mg, 200-400 mg, 200-375 mg, 200-350 mg, 200-300 mg, 200-400 mg, or 400-600 mg, or equivalents thereof, once a day.

In some embodiments, the effective dose of Azenosertib, or a pharmaceutically acceptable salt thereof, is 50 mg, 100 mg, 150 mg, 200 mg, 250 mg, 300 mg, 325 mg, 350 mg, 375 mg, 400 mg, 450 mg, 500 mg, 550 mg, or 600 mg, or equivalents thereof, once a day.

In some embodiments, the effective dose of Azenosertib, or a pharmaceutically acceptable salt thereof, is 200 mg, or equivalents thereof, once a day. In some embodiments, the effective dose of Azenosertib, or a pharmaceutically acceptable salt thereof, is 300 mg, or equivalents thereof, once a day. In some embodiments, the effective dose of Azenosertib, or a pharmaceutically acceptable salt thereof, is 350 mg, or equivalents thereof, once a day. In some embodiments, the effective dose of Azenosertib, or a pharmaceutically acceptable salt thereof, is 400 mg, or equivalents thereof, once a day. In some embodiments, the effective dose of Azenosertib, or a pharmaceutically acceptable salt thereof, is 450 mg, or equivalents thereof, once a day. In some embodiments, the effective dose of Azenosertib, or a pharmaceutically acceptable salt thereof, is 500 mg, or equivalents thereof, once a day. In some embodiments, the effective dose of Azenosertib, or a pharmaceutically acceptable salt thereof, is 600 mg, or equivalents thereof, once a day. In some embodiments, the effective dose of Azenosertib, or a pharmaceutically acceptable salt thereof, is 700 mg, or equivalents thereof, once a day. In some embodiments, the effective dose of Azenosertib, or a pharmaceutically acceptable salt thereof, is 800 mg, or equivalents thereof, once a day.

In some embodiments, the daily dose of Azenosertib, or a pharmaceutically acceptable salt thereof, is at or greater than 375 mg, 400 mg, 425 mg, 450 mg, 475 mg, 500 mg, 550 mg, 600 mg, 625 mg, 650 mg, 675 mg, 700 mg, 725 mg, 750 mg, 775 mg, 800 mg, or an equivalent thereof. In some embodiments, the present disclosure provides administration of a high dose of Azenosertib, or a pharmaceutically acceptable salt thereof, e.g. wherein the dose is or greater than 375 mg.

### Treatment Cycle

Methods of the present disclosure include administering Azenosertib, or a pharmaceutically acceptable salt thereof, and/or a second chemotherapeutic agent, or a pharmaceutically acceptable salt thereof, in a suitable dosing schedule. For example, the Azenosertib, or a pharmaceutically acceptable salt thereof, and/or a second chemotherapeutic agent, or a pharmaceutically acceptable salt thereof, described herein may be administered one or more times per day (for example once, twice or three times a day) for a certain number of days, followed by a period of days where no dose is given. This treatment cycle (including dosing days and no-dosing days) may then be repeated.

In some embodiments, a treatment cycle is a period of 3-28 days. In some embodiments, a treatment cycle is 5, 7, 10 or 14 days. In some embodiments, the treatment cycle is 21 days or 28 days. In some embodiments, the treatment cycle is repeated.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises one or more dosing weeks with each dosing week comprising at least three consecutive dosing days and at least one day without dosing. In some embodiments, the daily dose of Azenosertib, or a pharmaceutically acceptable salt thereof, is 400 mg. In some embodiments, the daily dose of Azenosertib, or a pharmaceutically acceptable salt thereof, is 450 mg.

In some aspects, the present disclosure provides administration of a high dose of Azenosertib, or a pharmaceutically acceptable salt thereof, e.g. between about 350 mg to about 800 mg once daily, or between about 175 mg to about 400 mg twice daily at an intermittent dosing regimen, e.g. 5 days administration (“on” days) followed by 2 days break (“off” days) i.e. 5/2, 4 days administration followed by 3 days break i.e. 4/3, or 3 days administration followed by 4 days off, i.e. 3/4, or 6 days administration followed by 1 day off i.e. 6/1. Alternatively, the intermittent dosing regimen of Azenosertib, or a pharmaceutically acceptable salt thereof, is also expressed as administering between about 350 mg to about 800 mg once daily, or between about 175 mg to about 400 mg twice daily at an intermittent frequency, e.g., 5 on/2 off, 4 on/2 off, 3 on/4 off, among others.

In some embodiments, the one or more dosing weeks are separated by at least one week of break. In some embodiments, the intermittent dosing regimen described herein (for example, of 7/0, 6/1, 5/2, 4/3 or 3/4) is carried out for 2 weeks followed by one week of break, or one week followed by one week of break, thereby achieving a high efficacy while increasing safety and tolerability in treating a cancer. In some embodiments, the intermittent dosing regimen described herein (for example, of 7/0, 5/2, 6/1, 4/3 or 3/4) is carried out for 3 weeks followed by one week of break, or one week followed by one week of break, thereby achieving a high efficacy while increasing safety and tolerability in treating a cancer. In some embodiments, the intermittent dosing regimen described herein (for example, of 7/0, 6/1, 5/2, 4/3 or 3/4) is carried out for greater than 3 weeks followed by one week of break, or one week followed by one week of break, thereby achieving a high efficacy while increasing safety and tolerability in treating a cancer.

In some aspects, provided herein is a method of treating cancer comprising administering to a subject in need thereof, a daily dose of Azenosertib, or a pharmaceutically acceptable salt thereof, at or greater than 100 mg, or an equivalent thereof, in accordance with an intermittent dosing cycle, wherein the intermittent dosing cycle comprises one or more dosing weeks with each dosing week comprises at least three consecutive dosing days and at least one day without dosing, followed by at least one week of break. In some embodiments, the daily dose of Azenosertib, or a pharmaceutically acceptable salt thereof, is at or greater than 100 mg, 125 mg, 150 mg, 175 mg, 200 mg, 225 mg, 250 mg, 275 mg, 300 mg, 325 mg, 350 mg, or an equivalent thereof. In some embodiments, Azenosertib, or a pharmaceutically acceptable salt thereof, is administered at a dose of about 200 mg once daily in an intermittent dosing regimen. In some embodiments, Azenosertib, or a pharmaceutically acceptable salt thereof, is administered at a dose of about 225 mg once daily in an intermittent dosing regimen. In some embodiments, Azenosertib, or a pharmaceutically acceptable salt thereof, is administered at a dose of about 250 mg once daily in an intermittent dosing regimen. In some embodiments, Azenosertib, or a pharmaceutically acceptable salt thereof, is administered at a dose of about 275 mg once daily in an intermittent dosing regimen. In some embodiments, Azenosertib, or a pharmaceutically acceptable salt thereof, is administered at a dose of greater than about 300 mg once daily in an intermittent dosing regimen. In some embodiments, Azenosertib, or a pharmaceutically acceptable salt thereof, is administered at a dose of about 300 mg once daily in an intermittent dosing regimen. In some embodiments, Azenosertib, or a pharmaceutically acceptable salt thereof, is administered at a dose of about 350 mg once daily in an intermittent dosing regimen.

In some embodiments, the daily dose of Azenosertib, or a pharmaceutically acceptable salt thereof, is at or greater than 375 mg, 400 mg, 425 mg, 450 mg, 475 mg, 500 mg, 550 mg, 600 mg, 625 mg, 650 mg, 675 mg, 700 mg, 725 mg, 750 mg, 775 mg, 800 mg, or an equivalent thereof. In some embodiments, the daily dose of Azenosertib, or a pharmaceutically acceptable salt thereof, is at or greater than 375 mg. In some embodiments, the daily dose of Azenosertib, or a pharmaceutically acceptable salt thereof, is at about 400 mg. In some embodiments, the daily dose of Azenosertib, or a pharmaceutically acceptable salt thereof, is at about 425 mg. In some embodiments, the daily dose of Azenosertib, or a pharmaceutically acceptable salt thereof, is at about 450 mg. In some embodiments, the daily dose of Azenosertib is at about 475 mg. In some embodiments, the daily dose of Azenosertib, or a pharmaceutically acceptable salt thereof, is at about 500 mg. In some embodiments, the daily dose of Azenosertib, or a pharmaceutically acceptable salt thereof, is at about 550 mg. In some embodiments, the daily dose of Azenosertib, or a pharmaceutically acceptable salt thereof, is at about 600 mg. In some embodiments, the daily dose of Azenosertib, or a pharmaceutically acceptable salt thereof, is at about 625 mg. In some embodiments, the daily dose of Azenosertib, or a pharmaceutically acceptable salt thereof, is at about 650 mg. In some embodiments, the daily dose of Azenosertib, or a pharmaceutically acceptable salt thereof, is at about 675 mg. In some embodiments, the daily dose of Azenosertib, or a pharmaceutically acceptable salt thereof, is at about 700 mg. In some embodiments, the daily dose of Azenosertib, or a pharmaceutically acceptable salt thereof, is at about 725 mg. In some embodiments, the daily dose of Azenosertib, or a pharmaceutically acceptable salt thereof, is at about 750 mg. In some embodiments, the daily dose of Azenosertib, or a pharmaceutically acceptable salt thereof, is at about 775 mg. In some embodiments, the daily dose of Azenosertib, or a pharmaceutically acceptable salt thereof, is at about 800 mg, or an equivalent thereof.

In some embodiments, the daily dose of Azenosertib, or a pharmaceutically acceptable salt thereof, is administered once per day.

In some embodiments, the daily dose of Azenosertib, or a pharmaceutically acceptable salt thereof, is divided into twice per day.

In some embodiments, each dosing week comprises at least four, five or six consecutive dosing days.

In some embodiments, each dosing week comprises five consecutive dosing days and two days without dosing.

In some embodiments, each dosing week comprises four consecutive dosing days and three days without dosing.

In some embodiments, each dosing week comprises three consecutive dosing days and four days without dosing.

In some embodiments, each dosing week comprises seven consecutive dosing days and seven days without dosing.

In some embodiments, each intermittent dosing cycle comprises between about 7 days to about 10 consecutive dosing days. In some embodiments, each intermittent dosing cycle comprises between about 8 consecutive dosing days. In some embodiments, each intermittent dosing cycle comprises between about 9 consecutive dosing days. In some embodiments, each intermittent dosing cycle comprises between about 10 consecutive dosing days.

In some embodiments, the intermittent dosing cycle comprises twenty-one consecutive dosing days and seven days without dosing.

In some embodiments, the intermittent dosing cycle comprises two consecutive dosing weeks.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises at least two consecutive dosing days and at least one day without dosing.

In some embodiments, the intermittent dosing cycle comprises at least three, four, five, six, seven, eight, nine, ten, eleven, twelve, thirteen or fourteen consecutive dosing days. In some embodiments, the intermittent dosing cycle comprises greater than fourteen consecutive dosing days. In some embodiments, the intermittent dosing cycle comprises twenty-one consecutive dosing days. In some embodiments, the intermittent dosing cycle comprises twenty-eight consecutive dosing days. In some embodiments, the intermittent dosing cycle comprises thirty-two consecutive dosing days. In some embodiments, the intermittent dosing cycle comprises forty-two consecutive dosing days.

In some embodiments, the intermittent dosing cycle comprises at least one two, three, four, five, six, or seven days without dosing. In some embodiments, the intermittent dosing cycle comprises one day without dosing. In some embodiments, the intermittent dosing cycle comprises between about two to seven days without dosing. In some embodiments, the intermittent dosing cycle comprises two days without dosing. In some embodiments, the intermittent dosing cycle comprises three days without dosing. In some embodiments, the intermittent dosing cycle comprises four days without dosing. In some embodiments, the intermittent dosing cycle comprises five days without dosing. In some embodiments, the intermittent dosing cycle comprises six days without dosing. In some embodiments, the intermittent dosing cycle comprises seven days with dosing.

In some embodiments, the intermittent dosing cycle includes consecutive dosing days of between about two to seven days (“on” days), followed by a period of break of between about one to seven days (“off” days).

In some embodiments, the intermittent dosing cycle comprises five consecutive dosing days and two days without dosing.

In some embodiments, the intermittent dosing cycle comprises four consecutive dosing days and three days without dosing.

In some embodiments, the intermittent dosing cycle comprises three consecutive dosing days and four days without dosing.

In some embodiments, the intermittent dosing cycle comprises six consecutive dosing days and one day without dosing.

In some embodiments, the intermittent dosing cycle comprises seven consecutive dosing days and seven days without dosing.

In some embodiments, the intermittent dosing cycle comprises fourteen consecutive dosing days and seven days without dosing.

In some embodiments, the intermittent dosing cycle comprises twenty-one consecutive dosing days and seven days without dosing.

In some embodiments, the daily dose of Azenosertib, or a pharmaceutically acceptable salt thereof, is at or greater than 375 mg, 400 mg, 425 mg, 450 mg, 475 mg, 500 mg, 550 mg, 600 mg, 625 mg, 650 mg, 675 mg, 700 mg, 725 mg, 750 mg, 775 mg, 800 mg, or an equivalent thereof. In some embodiments, the present disclosure provides administration of a high dose of Azenosertib, or a pharmaceutically acceptable salt thereof, e.g., wherein the dose is or greater than 375 mg. In some embodiments, Azenosertib, or a pharmaceutically acceptable salt thereof, is administered at a dose of about 400 mg once daily in an intermittent dosing regimen. In some embodiments, Azenosertib, or a pharmaceutically acceptable salt thereof, is administered at a dose of about 450 mg once daily in an intermittent dosing regimen. In some embodiments, Azenosertib, or a pharmaceutically acceptable salt thereof, is administered at a dose of about 500 mg once daily in an intermittent dosing regimen. In some embodiments, Azenosertib, or a pharmaceutically acceptable salt thereof, is administered at a dose of about 550 mg once daily in an intermittent dosing regimen. In some embodiments, Azenosertib, or a pharmaceutically acceptable salt thereof, is administered at a dose of about 600 mg once daily in an intermittent dosing regimen. In some embodiments, Azenosertib, or a pharmaceutically acceptable salt thereof, is administered at a dose of greater than about 600 mg once daily in an intermittent dosing regimen. In some embodiments, Azenosertib, or a pharmaceutically acceptable salt thereof, is administered at a dose of about 650 mg once daily in an intermittent dosing regimen. In some embodiments, Azenosertib, or a pharmaceutically acceptable salt thereof, is administered at a dose of about 700 mg once daily in an intermittent dosing regimen. In some embodiments, Azenosertib, or a pharmaceutically acceptable salt thereof, is administered at a dose of about 750 mg once daily in an intermittent dosing regimen. In some embodiments, Azenosertib, or a pharmaceutically acceptable salt thereof, is administered at a dose of about 775 mg once daily in an intermittent dosing regimen. In some embodiments, Azenosertib, or a pharmaceutically acceptable salt thereof, is administered at a dose of about 800 mg once daily in an intermittent dosing regimen.

In some embodiments, the daily dose of Azenosertib, or a pharmaceutically acceptable salt thereof, is administered once per day.

In some embodiments, the daily dose of Azenosertib, or a pharmaceutically acceptable salt thereof, is divided equally into twice per day.

In some embodiments, the daily dose of Azenosertib, or a pharmaceutically acceptable salt thereof, is divided equally into three doses per day. In some embodiments, the daily dose of Azenosertib, or a pharmaceutically acceptable salt thereof, is divided equally into four doses per day.

In some embodiments, the twice per day of Azenosertib, or a pharmaceutically acceptable salt thereof, is at or greater than 175 mg, 200 mg, 225 mg, 250 mg, 275 mg, 300 mg, 325 mg, 350 mg, 375 mg, 400 mg or an equivalent thereof.

In some aspects, provided herein is a method of treating cancer comprising administering to a subject in need thereof, a daily dose of Azenosertib, or a pharmaceutically acceptable salt thereof, at or greater than 400 mg, or an equivalent thereof, in accordance with an intermittent dosing cycle, wherein the intermittent dosing cycle comprises five consecutive dosing days and two consecutive days without dosing.

In some aspects, provided herein is a method of treating cancer comprising administering to a subject in need thereof, a daily dose of Azenosertib, or a pharmaceutically acceptable salt thereof, at or greater than 450 mg, or an equivalent thereof, in accordance with an intermittent dosing cycle, wherein the intermittent dosing cycle comprises five consecutive dosing days and two consecutive days without dosing.

In some aspects, provided herein is a method of treating cancer comprising administering to a subject in need thereof, a daily dose of Azenosertib, or a pharmaceutically acceptable salt thereof, at or greater than 400 mg, or an equivalent thereof, in accordance with an intermittent dosing cycle, wherein the intermittent dosing cycle comprises four consecutive dosing days and three consecutive days without dosing.

In some aspects, provided herein is a method of treating cancer comprising administering to a subject in need thereof, a daily dose of Azenosertib, or a pharmaceutically acceptable salt thereof, at or greater than 450 mg, or an equivalent thereof, in accordance with an intermittent dosing cycle, wherein the intermittent dosing cycle comprises four consecutive dosing days and three consecutive days without dosing.

In some embodiments, the intermittent dosing cycle is repeated.

In some embodiments, the method further comprises administering a second chemotherapeutic agent, or a pharmaceutically acceptable salt thereof, during the intermittent dosing cycle. Without wishing to be bound by any particular theory, administration of Azenosertib, or a pharmaceutically acceptable salt thereof, in combination with a second chemotherapeutic agent, or a pharmaceutically acceptable salt thereof, renders responsive a subject resistant to treatment by the second chemotherapeutic agent, or a pharmaceutically acceptable salt thereof, alone, or prevents or reduces toxicity by the agent, and/or improves efficacy of treatment as compared to monotherapy. Combination therapy using intermittent dosing cycle further benefits dosing by requiring, for example, a lower effective dose of the second chemotherapeutic agent, or a pharmaceutically acceptable salt thereof, and/or Azenosertib, or a pharmaceutically acceptable salt thereof.

In some embodiments, Azenosertib, or a pharmaceutically acceptable salt thereof, is administered in combination with one or more second chemotherapeutic agents (including pharmaceutically acceptable salts thereof) in an intermittent dosing cycle.

### Cancer Types

Methods of the present disclosure can be used to treat cancers.

In some embodiments, the cancer is glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colon cancer, colorectal cancer, endometrial cancer, endometrium cancer, esophagus cancer, eye cancer, gallbladder cancer, gastric cancer, gastrointestinal cancer, Hodgkin lymphoma, Non-Hodgkin lymphoma, hematological tumor, head cancer, hematologic malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, uterine sarcoma, uterine serous carcinoma (USC), uterine CS, vaginal cancer, vulvar cancer, Waldenstrom macroglobulinemia, Wilms tumor, solid tumor, or liquid tumor, HGSOC, invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymona, BRAF mutant metastatic colorectal cancer, uveal melanoma, high-grade serous ovarian, fallopian tube, or primary peritoneal cancer, BRAF V600E-mutated colorectal cancer, platinum-sensitive ovarian cancer, poly(ADP-ribose) polymerase inhibitor (PARPi)-resistant ovarian cancer, platinum-resistant ovarian cancer, platinum-refractory ovarian cancer, advanced pancreatic adenocarcinoma, pancreatic ductal adenocarcinoma, neuroendocrine tumor, neuroendocrine prostate cancer, pancreatic neuroendocrine tumor, and small cell lung cancer (SCLC), germ cell cancer, and stromal cancer.

In some embodiments, the subject has a cancer. In some embodiments, the cancer is breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer.

In some embodiments, the cancer is glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, hematologic malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma (USC), vaginal cancer, vulvar cancer, Waldenstrom macroglobulinemia, Wilms tumor, solid tumor, or liquid tumor.

In some embodiments, the cancer is a solid tumor or a hematologic malignancy. In some embodiments, the cancer is a solid tumor. In some embodiments, the solid tumor is selected from endometrial cancer, gallbladder cancer, ovarian cancer, HGSOC, endometrium cancer, melanoma, colorectal cancer, bladder cancer, breast cancer, invasive breast cancer, Triple Negative Breast Cancer (TNBC), prostate cancer, Lung cancer, NSCLC, SCLC esophagogastric cancer, gastric cancer, esophageal cancer, renal cancer, pRCC, ccRCC, chromophobe RCC, head and neck cancer, osteosarcoma cancer, pancreatic cancer, brain cancer, uterine CS, uterine cancer, adenoid cystic carcinoma (ACC), mesothelioma, cervical cancer, Diffuse large B cell lymphoma (DLBCL), non-Hodgkin lymphoma (NHL), liver cancer, glioblastoma (GBM), testicular cancer, Low-grade gliomas (LGGs), Pheochromocytoma and paraganglioma (PCPGs), cholangiocarcinoma, thyroid cancer, thymona, and uveal melanoma.

In some embodiments, the solid tumor is ovarian cancer. In some embodiments, the ovarian cancer is epithelial ovarian cancer, germ cell cancer, or stromal cancer. In some embodiments, the ovarian cancer is epithelial ovarian cancer. In some embodiments, the epithelial ovarian cancer is high grade serous ovarian cancer (HGSOC).

In some embodiments, the cancer is associated with a “homologous recombination deficiency,” “homologous recombination repair deficiency”, “homologous repair deficiency” or “HRD” which refers to a reduction or impairment of the homologous recombination process. In some embodiments, the cancer is associated with increased Cyclin E1 or Cyclin E1 biomarker levels (e.g., a CCNE1 gene-amplified cancer, a Cyclin E1 overexpressing/not CCNE1 gene-amplified cancer, a Cyclin E1-driven cancer).

In some embodiments, the cancer has homologous recombination deficiency (HRD) positive status. In some embodiments, the cancer is an HRD positive cancer selected from an ovarian cancer (including recurrent ovarian cancer), a breast cancer (such as triple-negative breast cancer and/or metastatic breast cancer), a prostate cancer (for example, a metastatic castration-resistant prostate cancer), a fallopian tube cancer, and a primary peritoneal cancer.

In some embodiments, the cancer is associated with an organ selected from adrenal gland, ampulla of vater, biliary tract, bladder/urinary tract, bone, bowel, breast, cervix, cns/brain, esophagus/stomach, eye, head and neck, kidney, liver, lung, lymphoid, myeloid, ovary/fallopian tube, pancreas, penis, peripheral nervous system, peritoneum, pleura, prostate, skin, soft tissue, testis, thymus, thyroid, uterus, vulva/vagina, adenocarcinoma in situ, extra gonadal germ cell tumor (EGCT), a mixed cancer type, high-grade neuroendocrine carcinoma of the ovary, high-grade serous fallopian tube cancer (HGSFT), ovarian choriocarcinoma, and ovarian carcinoma NOS (OCNOS).

In some embodiments, the cancer is ovarian cancer. In some embodiments, the cancer is uterine cancer. In some embodiments the cancer is breast cancer. In some embodiments, the cancer is prostate cancer.

In some embodiments, the cancer is a primary cancer that originates in the associated organ. In some embodiments, the cancer is primary peritoneal cancer.

In some embodiments, the cancer has metastasized to the associated organ.

In some embodiments, the cancer is a solid tumor or a hematologic malignancy.

In some embodiments, the cancer is a solid tumor.

In some embodiments, the solid tumor the solid tumor is selected from endometrial cancer, gallbladder cancer, ovarian cancer (e.g., HGSOC), endometrium cancer, melanoma, colorectal cancer, bladder cancer, breast cancer (e.g., invasive, Triple negative breast cancer (TNBC)), prostate cancer, Lung cancer (e.g., NSCLC, SCLC), esophagogastric cancer, gastric cancer, esophageal cancer, renal cancer (e.g., pRCC, ccRCC, chromophobe RCC), head and neck cancer, osteosarcoma cancer, pancreatic cancer, brain cancer, uterine CS, uterine cancer, adenoid cystic carcinoma (ACC), mesothelioma, cervical cancer, Diffuse large B cell lymphoma (DLBCL), non-Hodgkin lymphoma (NHL), liver cancer, glioblastoma (GBM), testicular cancer, Low-grade gliomas (LGGs), Pheochromocytoma and paraganglioma (PCPGs), cholangiocarcinoma, thyroid cancer, thymona, and uveal melanoma.

In some embodiments, the cancer is acute myeloid leukemia (AML).

In some embodiments, the tumor is neuroendocrine tumor, neuroendocrine prostate cancer and pancreatic neuroendocrine tumor.

In some embodiments, the solid tumor is ovarian cancer.

In some embodiments, the ovarian cancer is epithelial ovarian cancer, germ cell cancer, or stromal cancer.

In some embodiments, the ovarian cancer is epithelial ovarian cancer,

In some embodiments, the ovarian cancer is high grade serous ovarian cancer (HGSOC). In some embodiments, the ovarian cancer is platinum resistant ovarian cancer (PROC). In some embodiments, the ovarian cancer is cyclin E amplified ovarian cancer. In some embodiments, the ovarian cancer is a Cyclin E1-overexpressing cancer. In some embodiments, the ovarian cancer is a Cyclin E1-overexpressing/non amplified cancer.

In some embodiments, the cancer is platinum resistant. In some embodiments, the cancer is resistant to one or more chemotherapy regimens. In some embodiments, the cancer is refractory to one or more chemotherapy regimens.

In some embodiments, the cancer is uterine serous carcinoma (USC).

In some embodiments, the cancer is osteosarcoma.

In some embodiments, the solid tumor is a uterine serous carcinoma, ovarian cancer, peritoneal cancer, fallopian tube cancer, osteosarcoma, pancreatic cancer or BRAF mutant metastatic colorectal cancer.

In some embodiments, the cancer is acute myeloid leukemia (AML), acute lymphocytic leukemia (ALL), chronic myeloid leukemia (CML), chronic lymphocytic leukemia (CLL), chronic myelomonocytic leukemia (CM ML), cutaneous B-cell lymphoma, cutaneous T-cell lymphoma, Hodgkin's lymphoma, Non-Hodgkin's lymphoma, Waldenstrom macroglobulinemia, or multiple myeloma (MM).

In some embodiments, the cancer is a platinum refractory cancer or a platinum resistant cancer. In some embodiments, the cancer is a platinum resistant cancer. In some embodiments, the cancer is platinum resistant. In some embodiments, the cancer is resistant to one or more chemotherapy regimens. In some embodiments, the cancer is refractory to one or more chemotherapy regimens.

### Combination Therapies

The present disclosure provides methods of using Azenosertib, or a pharmaceutically acceptable salt thereof, in combination with one or more additional agents (e.g., combination therapy with chemotherapeutic agents). In one aspect, the present disclosure provides a method of treating cancer comprising, administering to a subject selected to have a predetermined Cyclin E1 status, or a Cyclin E1 biomarker level above a predetermined threshold, an effective dose of Azenosertib, or a pharmaceutically acceptable salt thereof, and a second chemotherapeutic agent, or a pharmaceutically acceptable salt thereof.

Combination therapy refers to a clinical intervention in which a subject is simultaneously exposed to two or more therapeutic regimens (e.g. Azenosertib, or a pharmaceutically acceptable salt thereof, and a second chemotherapeutic agent, or a pharmaceutically acceptable salt thereof). In some embodiments, the two or more chemotherapeutic regimens may be administered simultaneously. In some embodiments, the two or more chemotherapeutic regimens may be administered sequentially (e.g., a first regimen administered prior to administration of any doses of a second regimen). In some embodiments, the two or more chemotherapeutic regimens are administered in overlapping dosing regimens.

In some embodiments, combination therapy does not necessarily require that individual agents be administered together in a single composition (or even necessarily at the same time). In some embodiments, two or more therapeutic regimens (e.g. Azenosertib, or a pharmaceutically acceptable salt thereof, and a second chemotherapeutic agent, or a pharmaceutically acceptable salt thereof) of a combination therapy are administered to a subject separately, e.g., in separate compositions, via separate administration routes (e.g., one agent orally and another agent intravenously), and/or at different time points. In some embodiments, two or more chemotherapeutic agents may be administered together in a combination composition, or even in a combination compound (e.g., as part of a single chemical complex or covalent entity), via the same administration route, and/or at the same time.

In some embodiments, Azenosertib, or a pharmaceutically acceptable salt thereof, and the second chemotherapeutic, or a pharmaceutically acceptable salt thereof, are administered concurrently. In some embodiments, Azenosertib, or a pharmaceutically acceptable salt thereof, and the second chemotherapeutic, or a pharmaceutically acceptable salt thereof, are administered sequentially. In some embodiments, Azenosertib, or a pharmaceutically acceptable salt thereof, is administered prior to the second chemotherapeutic, or a pharmaceutically acceptable salt thereof. In other embodiments, Azenosertib, or a pharmaceutically acceptable salt thereof, is administered after the second chemotherapeutic, or a pharmaceutically acceptable salt thereof. In some embodiments, Azenosertib, or a pharmaceutically acceptable salt thereof, and the second chemotherapeutic, or a pharmaceutically acceptable salt thereof, are administered intermittently.

In some embodiments, the second chemotherapeutic agent is selected from bendamustine, bortezomib, carfilzomib, ixazomib, busulfan, carboplatin, cisplatin, cyclophosphamide, cladribine, paclitaxel, docetaxel, pegylated liposomal doxorubicin (PLD), dexamethasone, doxorubicin, gemcitabine, cytarabine, fludarabine, fluorouracil (5-FU), irinotecan, topotecan, temozolomide, triapine, azacitidine, 5-azacytidine, capecitabine, AraC-FdUMP[10] (CF-10), cladribine, etoposide, decitabine, daunorubicin, doxorubicin, ifosfamide, methotrexate, vincristine, hydroxyurea, and oxaliplatin, or a pharmaceutically acceptable salt of any of the foregoing.

In some embodiments, the cancer treatment is alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators, immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-Jun-N-terminal kinase (JNK) inhibitors, mitogen-activated protein kinase (MAPK) inhibitors, MDM2 inhibitors, MEK inhibitors, ERK inhibitors, MMP inhibitors, mT or inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinon, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin.

In some embodiments, the chemotherapeutic agents is carboplatin, cisplatin, paclitaxel, docetaxel, pegylated liposomal doxorubicin, doxorubicin, gemcitabine, cytarabine, fludarabine, fluorouracil (5-FU), irinotecan, topotecan, temozolomide, triapine, 5-azacytidine, capecitabine, AraC-FdUMP[10] (CF-10), cladribine, decitabine, hydroxyurea and oxaliplatin, or a pharmaceutically acceptable salt of any of the foregoing. In other embodiments, the chemotherapeutic agent is azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and vincristine, or a pharmaceutically acceptable salt of any of the foregoing.

In some embodiments, the second chemotherapeutic agent is carboplatin, paclitaxel, gemcitabine, or pegylated liposomal doxorubicin (PLD), or a pharmaceutically acceptable salt of any of the foregoing.

In some embodiments, the second chemotherapeutic agent is encorafenib, or a pharmaceutically acceptable salt thereof. In some embodiments, the second chemotherapeutic agent is cetuximab, or a pharmaceutically acceptable salt thereof. In some embodiments, the second chemotherapeutic agent consists of a combination of encorafenib and cetuximab, or a pharmaceutically acceptable salt of any of the foregoing.

### Dosing for a Second Chemotherapeutic Agent

In some embodiments, the second chemotherapeutic agent is carboplatin, paclitaxel, gemcitabine, or pegylated liposomal doxorubicin (PLD), or a pharmaceutically acceptable salt of any of the foregoing.

In some embodiments, the second chemotherapeutic agent is carboplatin, or a pharmaceutically acceptable salt thereof, and wherein carboplatin is administered intravenously at a dose ranging from 1-10 mg/mL*min for 15 minutes or longer once during the treatment cycle. In some embodiments, the second chemotherapeutic agent is carboplatin, or a pharmaceutically acceptable salt thereof, and wherein carboplatin, or a pharmaceutically acceptable salt thereof, is administered intravenously at a dose ranging from 3-6 mg/ml*min for 15 minutes or longer once during the treatment cycle. In some embodiments, the second chemotherapeutic agent is carboplatin, or a pharmaceutically acceptable salt thereof, and wherein carboplatin, or a pharmaceutically acceptable salt thereof, is administered intravenously at a dose ranging from 1-10 mg/ml*min, 2-10 mg/mL*min, 3-10 mg/mL*min, 4-10 mg/mL*min, 5-10 mg/mL*min, 6-10 mg/mL*min, 7-10 mg/ml*min, 8-10 mg/mL*min, 9-10 mg/ml*min, 2-8 mg/ml*min, 2-7 mg/mL*min, 3-7 mg/mL*min, 4-7 mg/ml*min, 5-7 mg/mL*min, 4-6 mg/ml*min, 2-6 mg/mL*min, 3-8 mg/ml*min, 9-10 mg/mL*min for 15 minutes or longer once during the treatment cycle.

In some embodiments, the second chemotherapeutic agent is PLD, or a pharmaceutically acceptable salt thereof, and wherein PLD, or a pharmaceutically acceptable salt thereof, is administered intravenously at a dose ranging from 10-100 mg/m² over 60 minutes once during the treatment cycle. In some embodiments, the second chemotherapeutic agent is PLD, or a pharmaceutically acceptable salt thereof, and wherein PLD, or a pharmaceutically acceptable salt thereof, is administered intravenously at a dose ranging from 5-50 mg/m² over 60 minutes once during the treatment cycle. In some embodiments, the second chemotherapeutic agent is PLD, or a pharmaceutically acceptable salt thereof, and wherein PLD, or a pharmaceutically acceptable salt thereof, is administered intravenously at a dose ranging from 10-40 mg/m² over 60 minutes once during the treatment cycle.

In some embodiments, the second chemotherapeutic agent is PLD, or a pharmaceutically acceptable salt thereof, and wherein PLD, or a pharmaceutically acceptable salt thereof, is administered intravenously at a dose ranging from 10-100 mg/m², 10-90 mg/m², 10-80 mg/m², 10-70 mg/m², 10-60 mg/m², 10-50 mg/m², 10-40 mg/m², 10-30 mg/m², 10-20 mg/m², 20-90 mg/m², 30-90 mg/m², 40-90 mg/m², 50-90 mg/m², 60-90 mg/m², 70-90 mg/m², 20-80 mg/m², 20-70 mg/m², 20-60 mg/m², 20-50 mg/m², 20-40 mg/m², or 30-40 mg/m² over 60 minutes once during the treatment cycle.

In some embodiments, the second chemotherapeutic agent is paclitaxel, or a pharmaceutically acceptable salt thereof, and wherein paclitaxel, or a pharmaceutically acceptable salt thereof, is administered intravenously at a dose ranging from 10-120 mg/m² over 60 minutes (+10 minutes) three times during the treatment cycle. In some embodiments, the second chemotherapeutic agent is paclitaxel, or a pharmaceutically acceptable salt thereof, and wherein paclitaxel, or a pharmaceutically acceptable salt thereof, is administered intravenously at a dose ranging from 10-100 mg/m², 20-100 mg/m², 30-100 mg/m², 40-100 mg/m², 50-100 mg/m², 60-100 mg/m², 70-100 mg/m², 80-100 mg/m², 90-100 mg/m², 10-90 mg/m², 10-80 mg/m², 10-70 mg/m², 10-60 mg/m², 10-50 mg/m², 10-40 mg/m², 10-30 mg/m², 30-70 mg/m², 40-70 mg/m², 50-70 mg/m², 60-70 mg/m², 30-90 mg/m², 30-80 mg/m² administered up to 3 hours, three times during the treatment cycle.

In some embodiments, the second chemotherapeutic agent is paclitaxel, or a pharmaceutically acceptable salt thereof, and wherein paclitaxel, or a pharmaceutically acceptable salt thereof, is administered intravenously at a dose ranging from 40-100 mg/m² administered up to 3 hours up to three times during treatment cycles.

In some embodiments, the second chemotherapeutic agent is gemcitabine, or a pharmaceutically acceptable salt thereof, and wherein gemcitabine, or a pharmaceutically acceptable salt thereof, is administered intravenously at a dose ranging from 500-1500 mg/m² over 15 minutes longer once during the treatment cycle.

In some embodiments, the second chemotherapeutic agent is gemcitabine, or a pharmaceutically acceptable salt thereof, and wherein gemcitabine, or a pharmaceutically acceptable salt thereof, is administered intravenously at a dose ranging from 100 to 1000 mg/m², 100 to 1000 mg/m², 100 to 900 mg/m², 100 to 800 mg/m², 100 to 700 mg/m², 100 to 600 mg/m², 100 to 500 mg/m², 100 to 400 mg/m², 100 to 300 mg/m², 100 to 200 mg/m², 200 to 1000 mg/m², 300 to 1000 mg/m², 400 to 1000 mg/m², 500 to 1000 mg/m², 600 to 1000 mg/m², 700 to 1000 mg/m², 800 to 1000 mg/m², 200 to 800 mg/m², 200 to 700 mg/m², 200 to 600 mg/m², 200 to 500 mg/m², 300 to 900 mg/m², 300 to 800 mg/m², 400 to 700 mg/m², 500 to 700 mg/m², 500 to 800 mg/m², 600 to 900 mg/m² over 15 min or longer up to 3 times during the treatment cycle.

In some embodiments, the second chemotherapeutic agent is gemcitabine, or a pharmaceutically acceptable salt thereof, and wherein gemcitabine, or a pharmaceutically acceptable salt thereof, is administered intravenously at a dose ranging from 100 to 1000 mg/m² over 15 min or longer up to 3 times during the treatment cycle.

### Responsiveness

In some embodiments, the treatment methods described herein result in a response rate at or greater than 10%, 15%, 20%, 25%, 30%, 35%, 40%, 45%, or 50%. In some embodiments, the response rate is measured by complete response (CR), partial response (PR), CA-125 50% response, or combination thereof. In some embodiments, response is determined based on progression free survival. In some embodiments, response is determined based on tumor response. In some embodiments, response is determined based on clinical benefit rate (CBR). In some embodiments, In some embodiments, response is determined based on disease control rate (DCR). In some embodiments, response is determined based on overall survival (OS).

Progression free survival (PFS) refers to the time period for which a subject having a disease (e.g. cancer) survives, without a significant worsening of the disease state. Progression free survival may be assessed as a period of time in which there is no progression of tumor growth and/or wherein the disease status of a subject is not determined to be a progressive disease. In embodiments, progression free survival of a subject having cancer is assessed by evaluating tumor size, tumor number, and/or metastasis.

In some embodiments, the treatment results in progression-free survival (PFS) of 5 months, 6 months, 7 months, 8 months, 9 months, 10 months, 11 months, 12 months or longer. In some embodiments, the treatment results in progression-free survival (PFS) of 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or longer. In some embodiments, the treatment results in median progression-free survival (mPFS) of 1 year, 1.5 years, 2 years, 2.5 years, or longer.

As used herein, the term “progression” of tumor growth or a “progressive disease” (PD) as used herein in reference to cancer status indicates an increase in the sum of the diameters of the target tumors. Progression for the purposes of determining progression free survival may also be determined if at least one of the following criteria is met: 1) tumor assessment by CT/MRI unequivocally shows progressive disease according to RECIST 1.1 criteria; or 2) additional diagnostic tests (e.g. histology/cytology, ultrasound techniques, endoscopy, positron emission tomography) identify new tumors or determine existing tumors qualify for unequivocal progressive disease and/or CA-125-progression according to Gynecologic Cancer Intergroup (GCIG)-criteria (see Rustin et al., Int J Gynecol Cancer 2011; 21:419-423 which is incorporated herein in its entirety); 3) definitive clinical signs and symptoms of PD unrelated to non-malignant or iatrogenic causes ([i] intractable cancer-related pain; [ii] malignant bowel obstruction/worsening dysfunction; or [iii] unequivocal symptomatic worsening of ascites or pleural effusion) and/or CA-125-progression according to GCIG-criteria.

As used herein, the term “partial response” or “PR” refers to a decrease in tumor progression in a subject as indicated by a decrease in the sum of the diameters of the target tumors, taking as reference the baseline sum diameters. In embodiments, PR refers to at least a 30% decrease in the sum of diameters, taking as reference the baseline sum diameters. Exemplary methods for evaluating partial response are identified by RECIST guidelines. See E. A. Eisenhauer, et al., “New response evaluation criteria in solid tumors: Revised RECIST guideline (version 1.1.),” Eur. J. of Cancer, 45:228-247 (2009).

As used herein, “stabilization” of tumor growth or a “stable disease” (SD) refers to neither sufficient shrinkage to qualify for PR nor sufficient increase to qualify for PD. In embodiments, stabilization refers to a less than 30%, 25%, 20%, 15%, 10% or 5% change (increase or decrease) in the sum of the diameters of the target tumors, taking as reference the baseline sum diameters. Exemplary methods for evaluating stabilization of tumor growth or a stable disease are identified by RECIST guidelines. See E. A. Eisenhauer, et al., “New response evaluation criteria in solid tumors: Revised RECIST guideline (version 1.1.),” Eur. J. of Cancer, 45:228-247 (2009).

As used herein, the term “complete response” or “CR” is used to mean the disappearance of all or substantially all target lesions. In embodiments, CR refers to an 80%, 85%, 90%, 91%, 92%, 93%, 94%, 95%, 96%, 97%, 98%, 99% or 100% decrease in the sum of the diameters of the target tumors (i.e. loss of tumors), taking as reference the baseline sum diameters. In embodiments, CR indicates that less than 10%, 9%, 8%, 7%, 6%, 5%, 4%, 3%, 2%, 1% or less of the total lesion diameter remains after treatment. Exemplary methods for evaluating complete response are identified by RECIST guidelines. See E. A. Eisenhauer, et al., “New response evaluation criteria in solid tumors: Revised RECIST guideline (version 1.1.),” Eur. J. of Cancer, 45:228-247 (2009).

### EXAMPLES

Additional embodiments are disclosed in further detail in the following examples, which are not in any way intended to limit the scope of the claims.

### Example 1. Cancer Cell Lines with Cyclin E1 Overexpression are Sensitive to Azenosertib

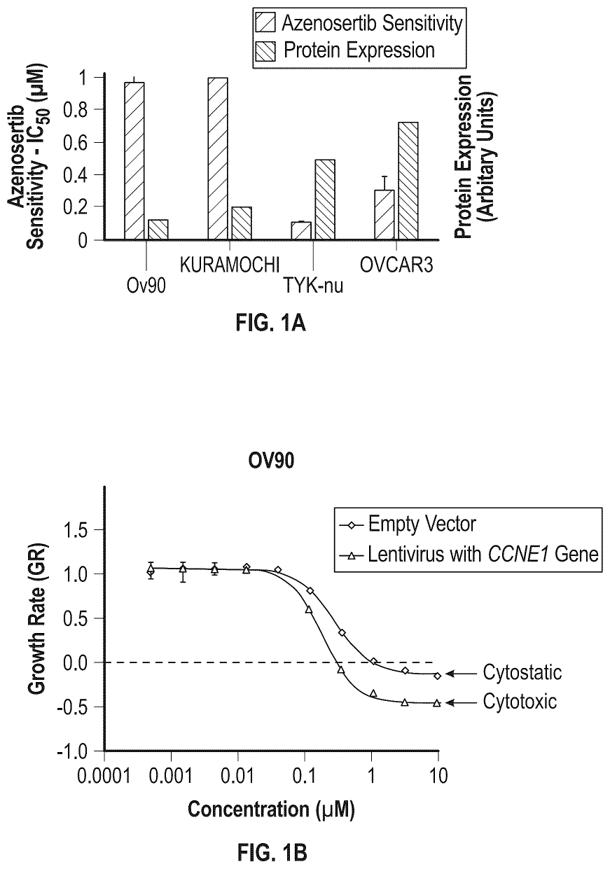

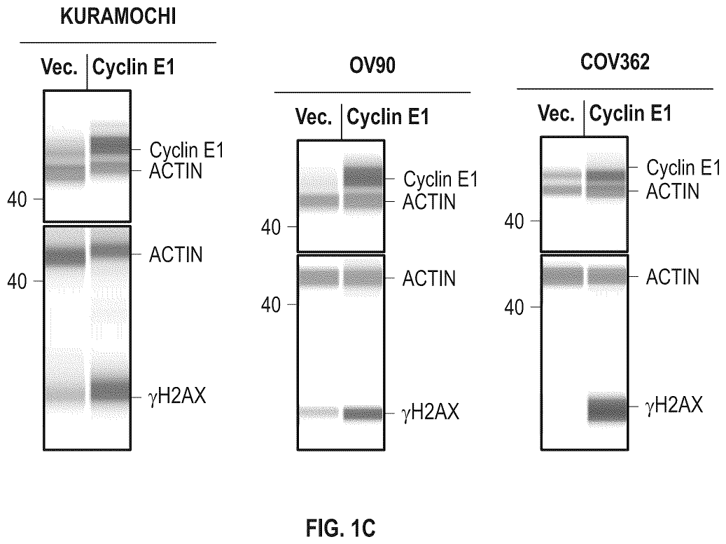

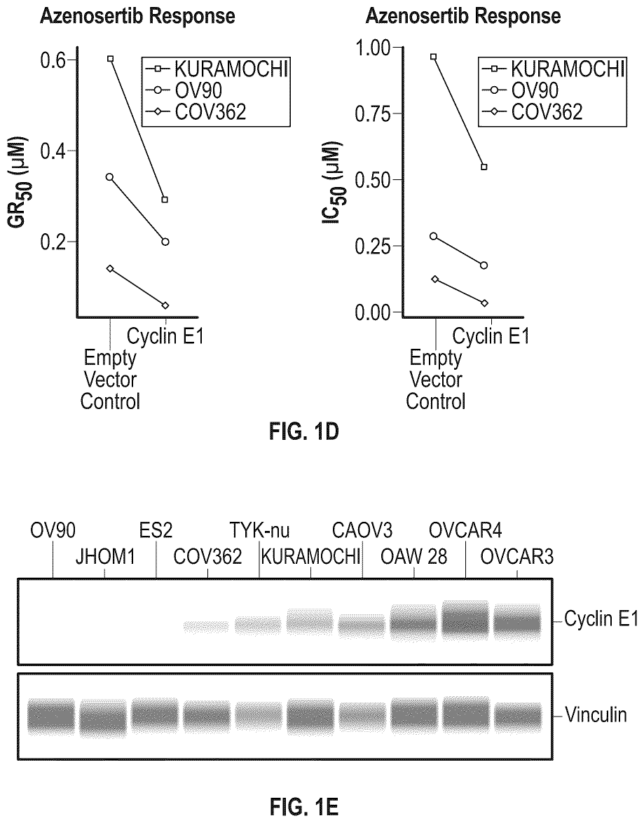

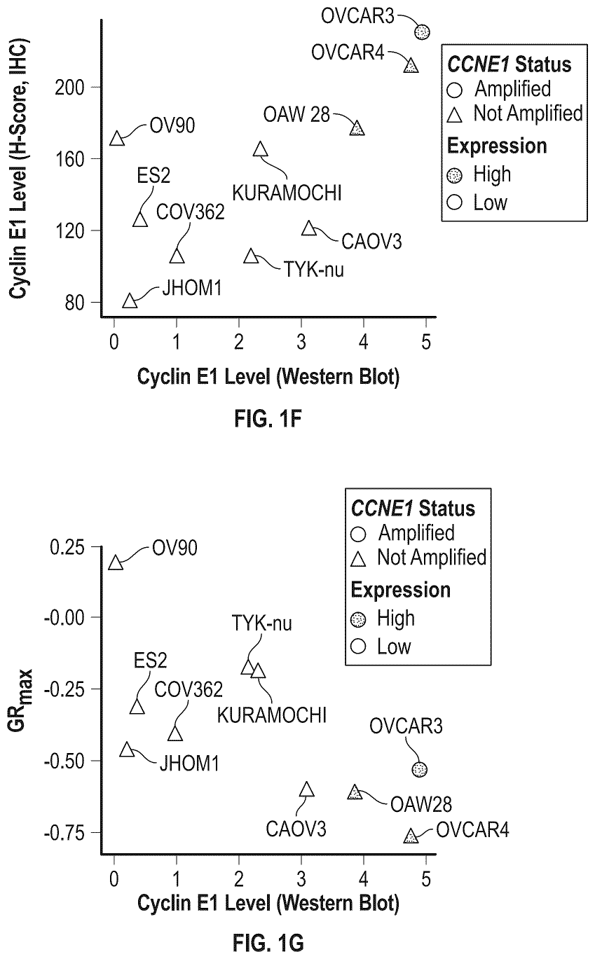

Cyclin E1 (encoded by the CCNE1 gene) protein overexpression is associated with increased sensitivity to Wee1 inhibitor Azenosertib in ovarian cancer cell lines. As shown in FIG. 1A, Azenosertib sensitivity correlates with Cyclin E1 protein expression in OV90, Kuramochi, TYK-nu and OVCAR3 cells. Cells were assessed for protein expression and Azenosertib sensitivity by CellTiter Glo after 96 hours of culture.

To confirm whether Cyclin E1 expression levels were responsible for increased sensitivity to Azenosertib, OV90, Kuramochi and COV362 cells (low endogenous expression levels of Cyclin E1) were transduced with a lentiviral vector carrying the CCNE1 gene or with an empty vector control. Stable OV90, Kuramochi and COV362 cell lines were established by puromycin selection. Cyclin E1 overexpression in the stable OV90 cell line compared to empty vector control cells was confirmed by Western blotting (FIG. 1C). As shown in FIG. 1B, overexpression increased sensitivity to Azenosertib. Sensitization of HGSOC cell lines by overexpression of Cyclin E1 to Azenosertib was assessed by plotting the growth-rate (GR) inhibition in FIG. 1D. It was observed that Cyclin E1 overexpression sensitized HGSOC cell lines to Azenosertib by further decreasing growth rate and cell viability compared to control empty vector cell lines (FIG. 1D). GR₅₀ and IC₅₀ values of Azenosertib were determined by CellTiter-Glo (CTG) assay and the GR calculator.

Similar assays were performed in other cell lines—for example, JOM1, ES2, TYK-nu, CAOV3, OAW28, OVCAR4, OVCAR3. OVCAR3 (CCNE1-amplified, CN=12), OAW28 and OVCAR4 cells express high levels of Cyclin E1 which was determined by Western blot (WB) and immunohistochemistry (IHC). Cyclin E1 expression was normalized to Vinculin. FIG. 1E shows the Cyclin E1 protein overexpression state by Western blot. These values are plotted by using H-score as a function of Cyclin E1 levels (FIG. 1F). FIG. 1G shows a graph of GR value as a function of Cyclin E1 levels.

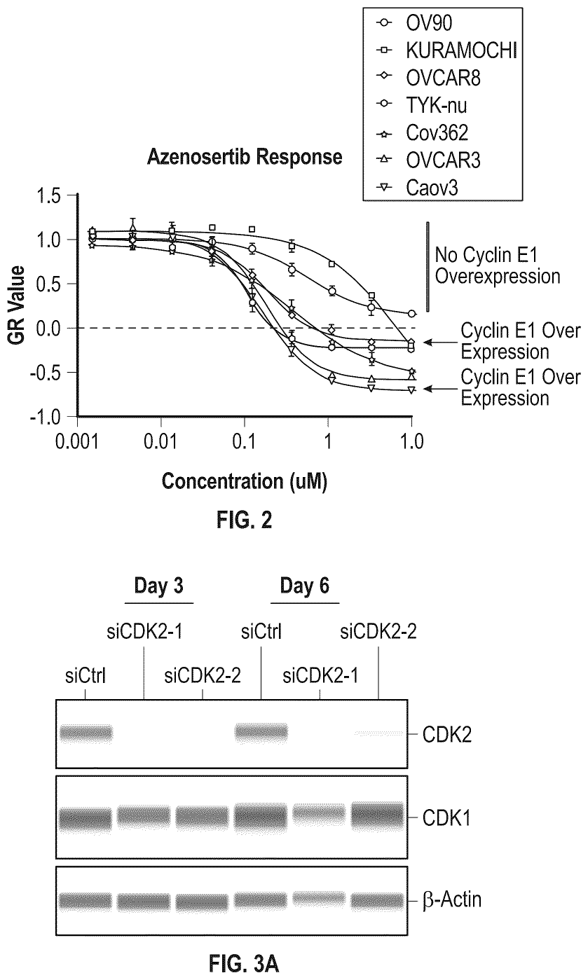

HGSOC cells were assessed for growth rate inhibition (GR) in the presence of Azenosertib (relative to an untreated control). GR value is independent of cell division rate and was determined as described in Hafner et al. (Curr. Protoc. Chem. Biol. 9, 96-116 (2017)). HGSOC cells with high Cyclin E1 protein expression were amongst the most sensitive to Azenosertib. Low or no Cyclin E1-expressing cells demonstrate reduced sensitivity or resistance to Azenosertib. (FIG. 2). An inhibitory effect was observed in OV90 and Kuramochi cells, a cytotoxic effect was observed in ES2 and TYK-nu cells, and a cytotoxic effect was observed in COV362, JHOMI, OAW28, OVCAR3, CAOV3, and OVCAR4 cells treated with Azenosertib. Ovarian cancer cell lines overexpressing Cyclin E1 protein (Cyclin E1-high) were more sensitive to Azenosertib than ovarian cancer cell lines with lower levels of Cyclin E1 protein (Cyclin E1-low) inducing significant cytotoxic effects (GRₘₐₓ<−0.5). In Cyclin E1-low ovarian cancer cell lines, overexpression of Cyclin E1 by lentivirus increased sensitivity to Azenosertib.

Taken together, these data suggest that high Cyclin E1 protein expression is associated with sensitivity to Azenosertib, and artificial overexpression of Cyclin E1 in cell lines with low endogenous Cyclin E1 expression sensitize the cells to Azenosertib.

### Example 2. CDK2-Dependent Sensitivity of Cyclin E1-High HGSOC to Azenosertib

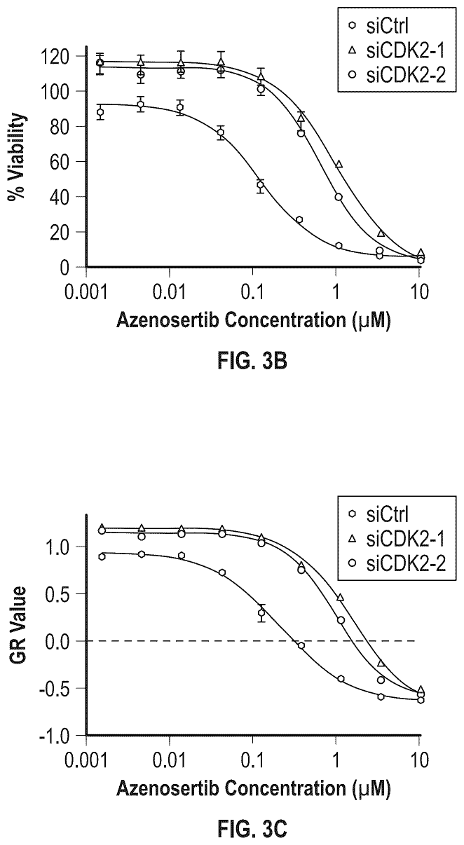

The effect of CDK2 in sensitization of Cyclin E1-high HGSOC to Azenosertib was assessed by using two different siRNA to knock down the CDK2 protein expression. The down regulation of CDK2 was assessed by Western blot (FIG. 3A), 3 and 6 days after transfection. After 3 days of CDK2 siRNA s transfection, OVCAR4 cells were treated with Azenosertib for 3 days. Percent viability and growth rate values were determined by CellTiter-Glo assay and the growth rate calculator. FIG. 3B and FIG. 3C show the percent viability and growth-rate reduction after treatment with Azenosertib after siRNA treatments. It was observed that the sensitivity of Cyclin E1-high HGSOC to Azenosertib is dependent on CDK2.

These data provide foundational details on the mechanistic basis of Cyclin E1 sensitization to Wee1 inhibition, including that Cyclin E1 protein overexpression results in accumulation of replication stress biomarkers and that Azenosertib sensitivity is mediated by CDK2 activity.

### Example 3. Effects of Azenosertib in a Cyclin E1-High Tumor Mouse Model with Increased Replication Stress

This Example demonstrates that greater anti-tumor effects of Azenosertib in a Cyclin E1-high tumor model are associated with increased replication stress.

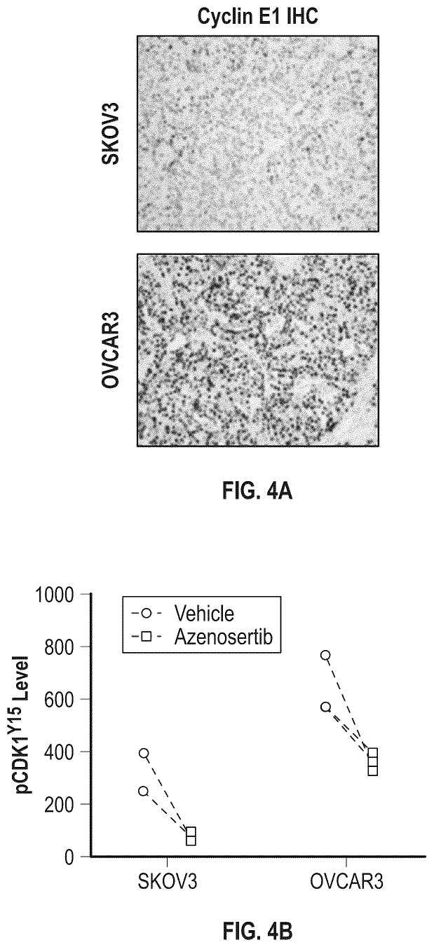

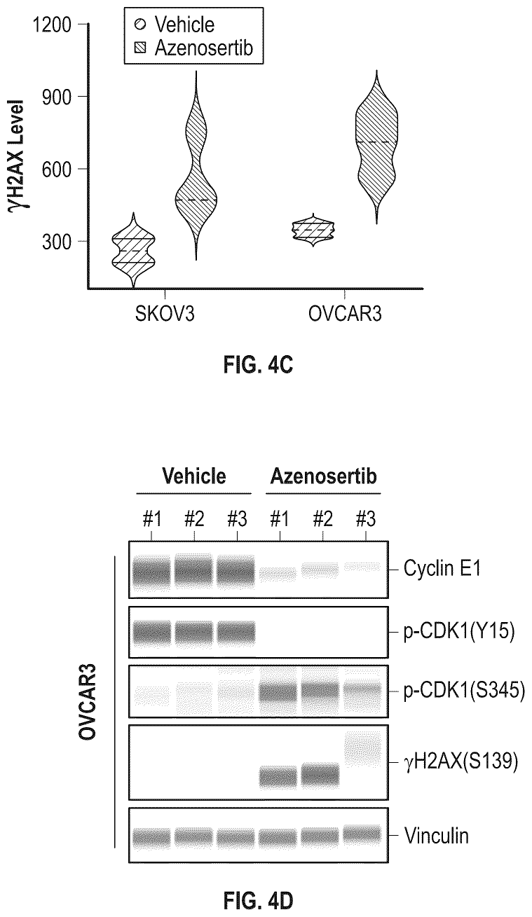

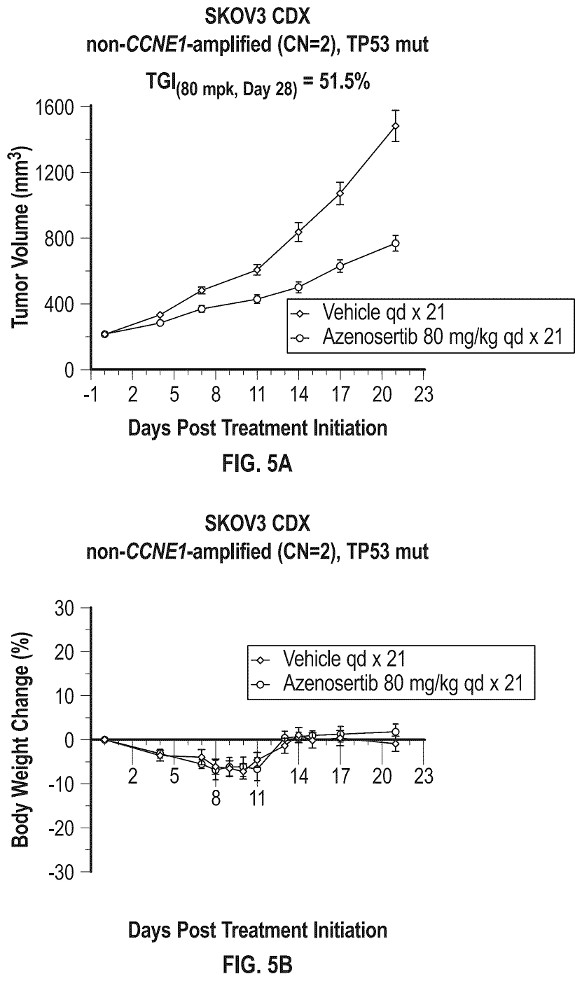

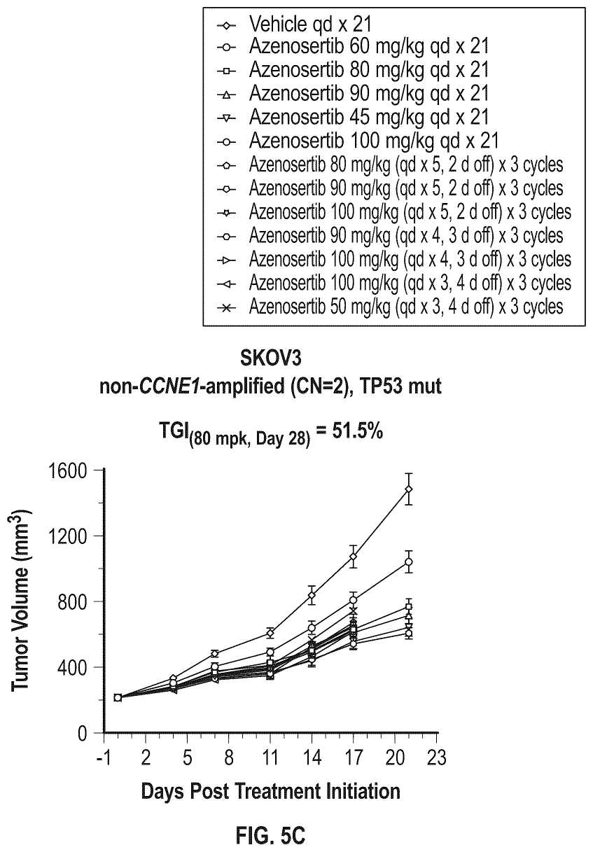

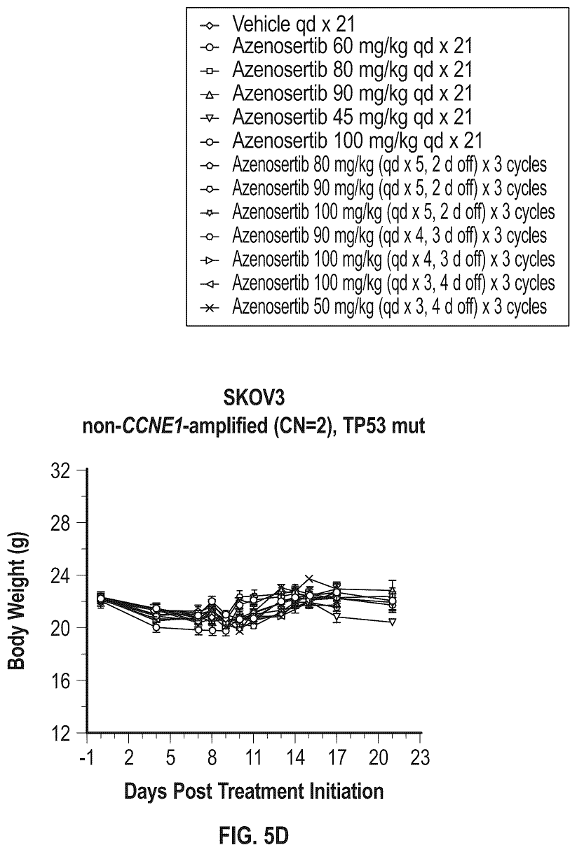

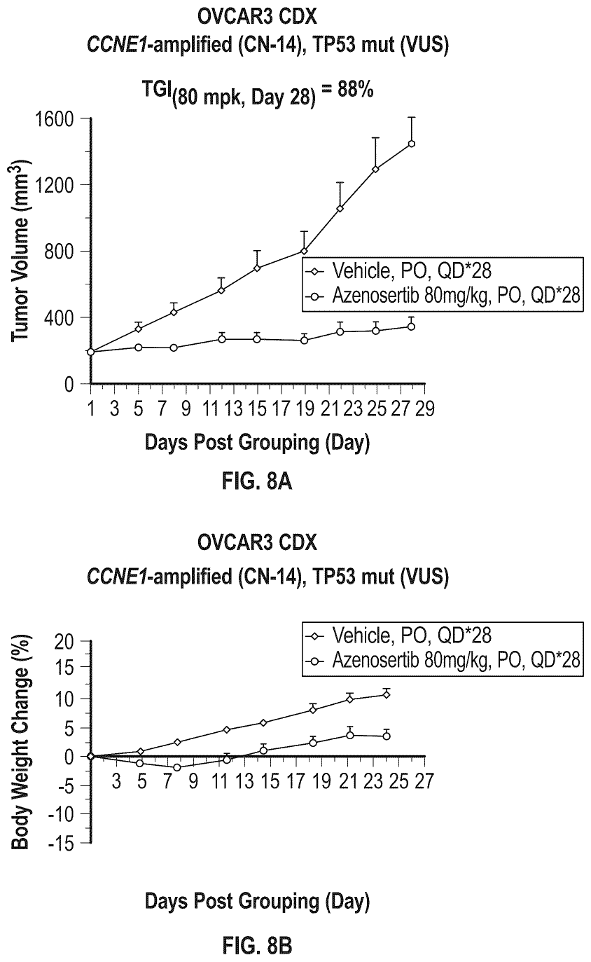

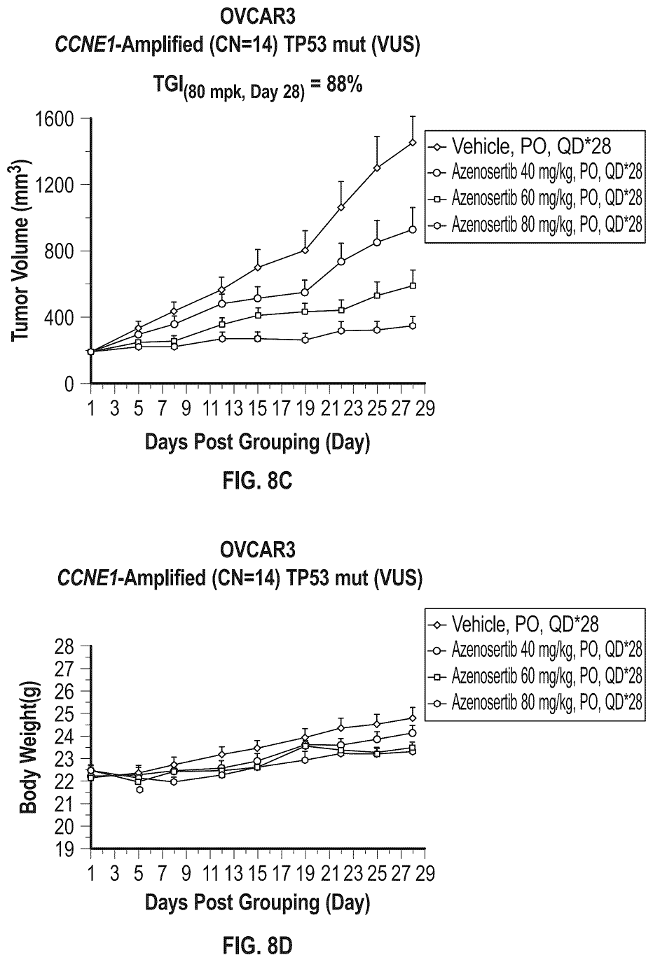

NOD/SCID mice (SKOV3-Cyclin E1-low and OVCAR3-Cyclin E1-high) or BALB/c nude mice (HCC1806) bearing corresponding tumors were dosed orally every day for the time as shown in FIGS. 5A and 8A. Treatment was well-tolerated and the change in tumor volume was plotted against time of administration. Baseline Cyclin E1 protein expression of each model was examined by IHC and shown in FIG. 4A. Target engagement of Azenosertib (decreased pCDK1) and γH2AX were examined by IHC in tumors after 12 hrs of Azenosertib treatment in SKOV3-Cyclin E1-low and OVCAR3-Cyclin E1-high cells. Y-axis represents the sum of H-score evaluated by 3 independent pathologists (FIGS. 4B and 4C). Replication stress markers pCHK1 and γH2AX were determined by Western Blot in OVCAR3 tumor samples with or without Azenosertib treatment for 12 hrs (FIG. 4D).

### Example 4. Mice Models with CCNE1-Amplified Cell Line-Derived Xenografts (CDX) Demonstrate Increased Sensitivity to Azenosertib

This example demonstrates that treatment with Azenosertib results in reduction of tumor volume and increased CCNE1 gene amplification correlates with increased sensitivity in CDX models.

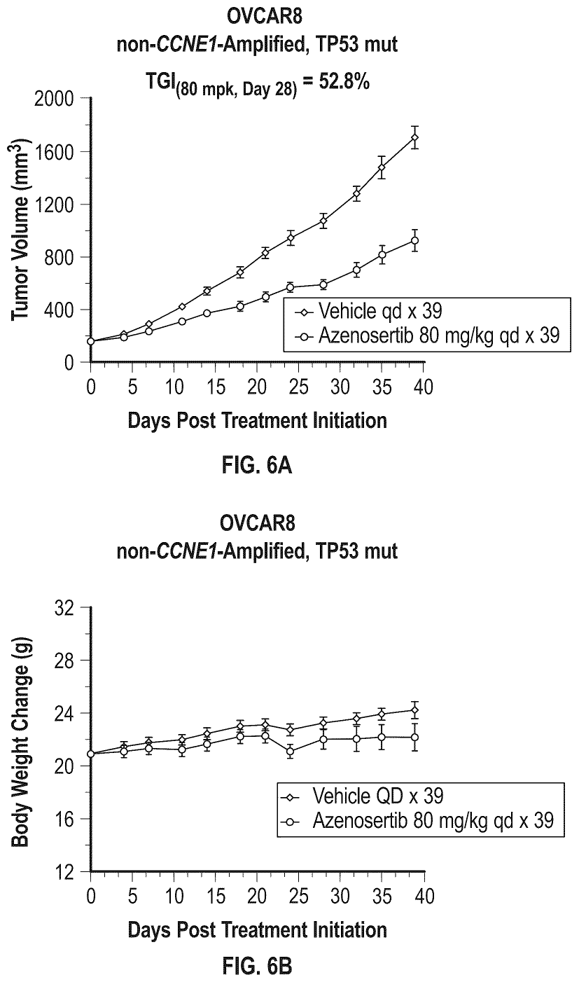

Mice were inoculated with SKOV3 cells (non-CCNE1-amplified, wild-type CCNE1 CN=2) on the right flank with the single cell suspension of 95% viable tumor cells (1×10⁷) in 100 μL McCoy's 5a and Matrigel mixture (1:1 ratio) without serum for the tumor development. Animals were randomized and treated with vehicle (20% HP-β-CD) or 80 mg/kg Azenosertib daily for 21 days when the mean tumor volume reached 215 mm³. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. Azenosertib treated mice showed reduction in tumor growth compared to vehicle treated control animals (FIGS. 5A and 5B). Additional dosages and schedules were included as summarized below in Table 1 and shown in FIGS. 5C and 5D. Treatment with 80 mg/kg Azenosertib daily resulted in an average tumor growth inhibition of TGI_{(80 mpk, Day 28)} of 51.5% at Day 28. Similarly, OVCAR8 (non=CCNE1-amplified) CDX models were administered were treated with 80 mg/kg Azenosertib daily for 39 days and demonstrated an average tumor growth inhibition of TGI_{(80 mpk, Day 28)} of 52.8%. (FIGS. 6A and 6B). These results demonstrate that in non-CCNE1-amplified ovarian models, Azenosertib moderately inhibits tumor growth.

TABLE 1
Azenosertib dose and schedule

| Azenosertib Dose | Schedule |
|---|---|
| 60 mg/kg | QD × 21 |
| 80 mg/kg | QD × 21 |
| 90 mg/kg | QD × 21 |
| 45 mg/kg | BID × 21 |
| 100 mg/kg | QD × 21 |
| 80 mg/kg | QD × 5, 2 days off × 3 cycles |
| 90 mg/kg | QD × 5, 2 days off × 3 cycles |
| 100 mg/kg | QD × 5, 2 days off × 3 cycles |
| 90 mg/kg | QD × 5, 2 days off × 3 cycles |
| 100 mg/kg | QD × 4, 3 days off × 3 cycles |
| 100 mg/kg | QD × 3, 4 days off × 3 cycles |
| 50 mg/kg | BID × 3, 4 days off × 3 cycles |

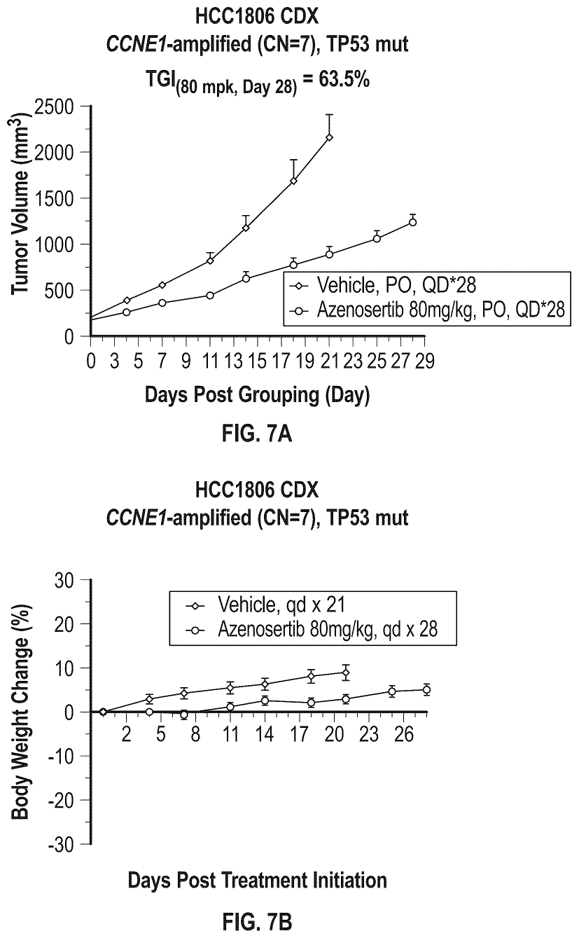

1×10⁶ HCC1806 human triple negative breast cancer cells (CCNE1-amplified, CN=7) were inoculated into the right flank of ten 6-8-week-old female BALB/c nude mice. Animals were randomized and treatment started when the mean tumor volume reached 155 mm³. Animals were treated with vehicle (20% HP-β-CD) or 80 mg/kg Azenosertib daily and tumor volumes and body weight were measured twice weekly. Compared against mice treated with vehicle, Azenosertib-treated mice showed significantly reduced tumor growth (63.5%) after 28 days (FIGS. 7A and 7B).

6-8 weeks old female NOD/SCID mice were inoculated with OVCAR-3 cells (CCNE1-amplified, CN=14) into the right flank. Animals were randomized and treated with 80 mg/kg Azenosertib vehicle (20% HP-β-CD) or 80 mg/kg Azenosertib daily once the mean tumor volume reached 111 mm³. Tumor volumes and body weight were measured twice weekly. Azenosertib treated t mice showed significantly reduced tumor growth (TGI_{(80 mpk, Day 28)}) of 88% after 28 days compared to vehicle control (FIGS. 8A and 8B). Reduction in tumor growth was also observed in animals treated with 40 mg/kg Azenosertib daily, 60 mg/kg Azenosertib daily, 80 mg/kg Azenosertib daily. (FIGS. 8C and 8D).

Taken together, these results indicate that CCNE1 gene amplification (e.g., high copy number) leads to increased efficacy of Azenosertib treatment (e.g., improved tumor growth inhibition).

### Example 5. CCNE1 Gene Amplification Increases Sensitivity to Azenosertib in Combination with a Second Chemotherapeutic Agent

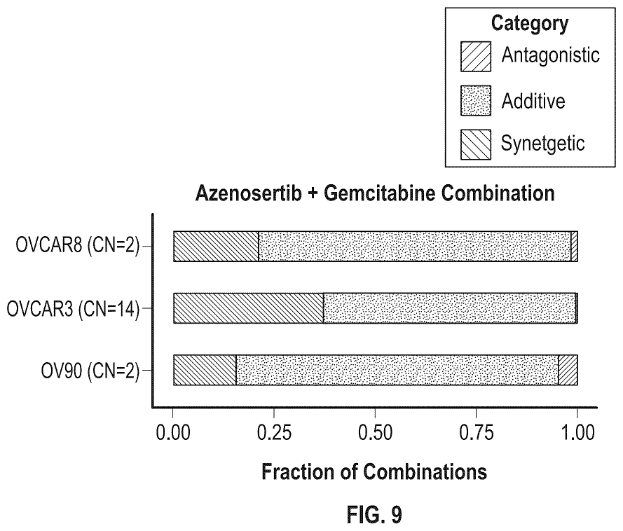

Ovarian cancer cell lines were evaluated for sensitivity to Azenosertib in combination with gemcitabine. As shown in FIG. 9, 37% of the tested combination conditions were synergetic in OVCAR3 (CCNE1-amplified, CN=14), compared to 16% and 21% in OV90 (non-CCNE1-amplified, CN=2) and OVCAR8 (non-CCNE1-amplified, CN=2), respectively.

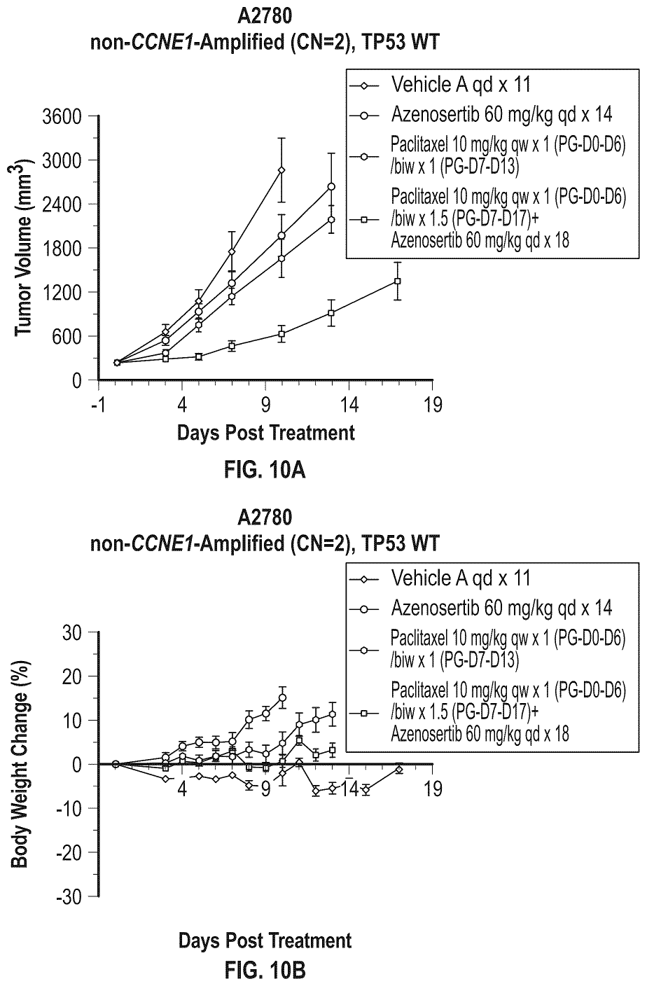

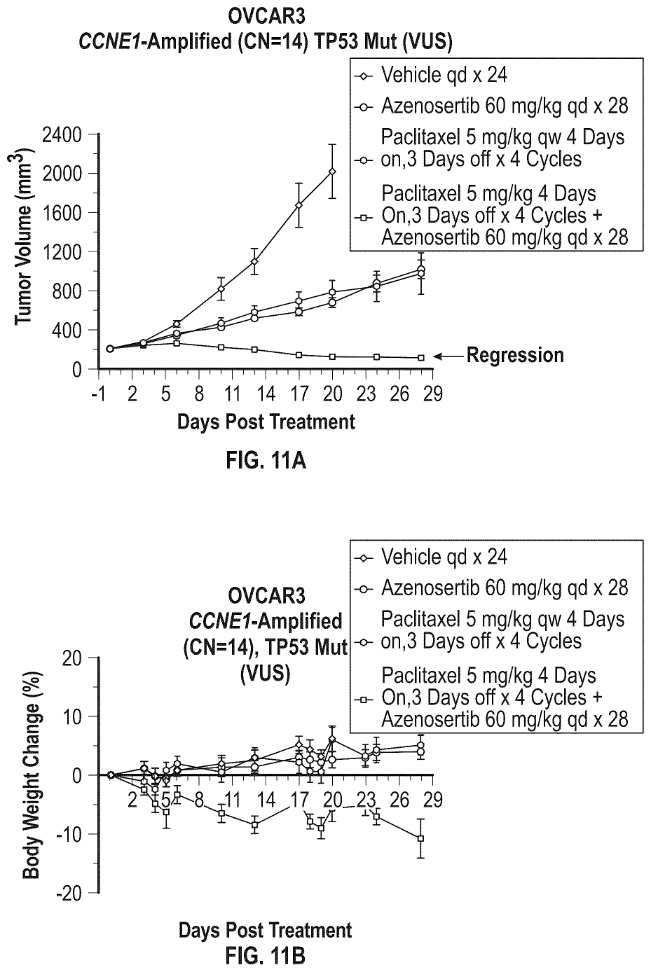

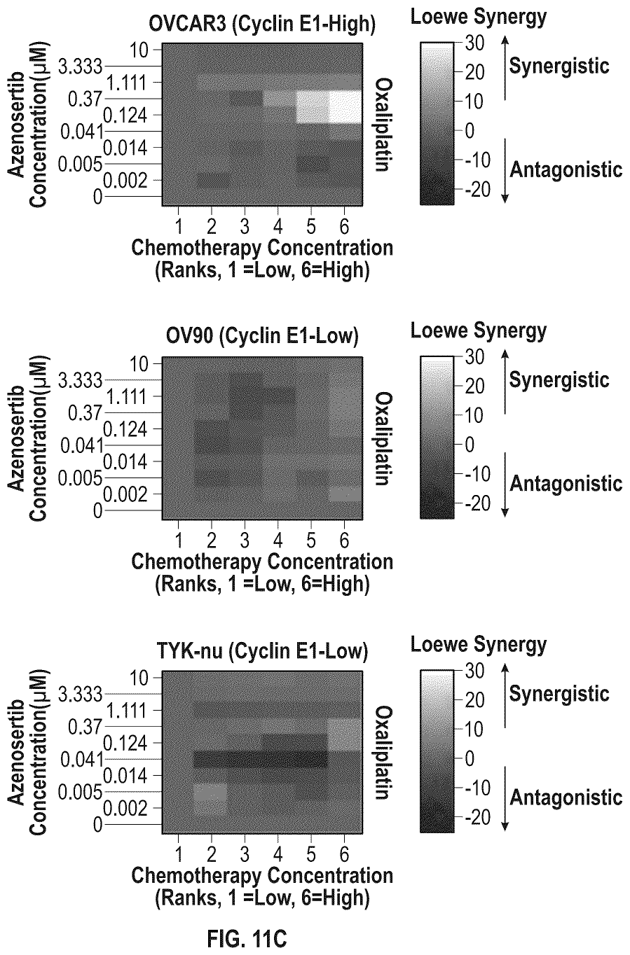

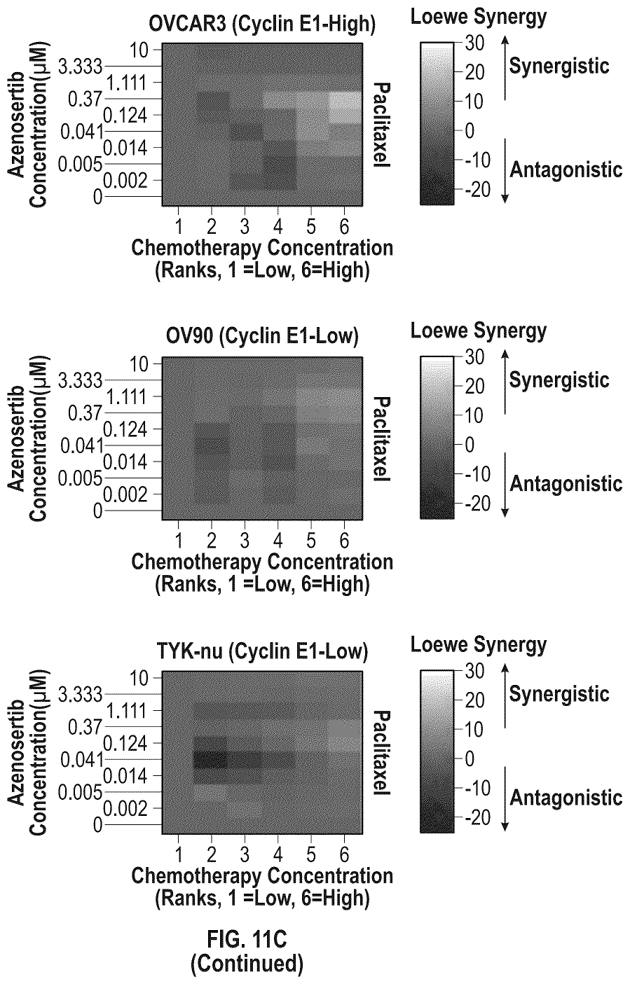

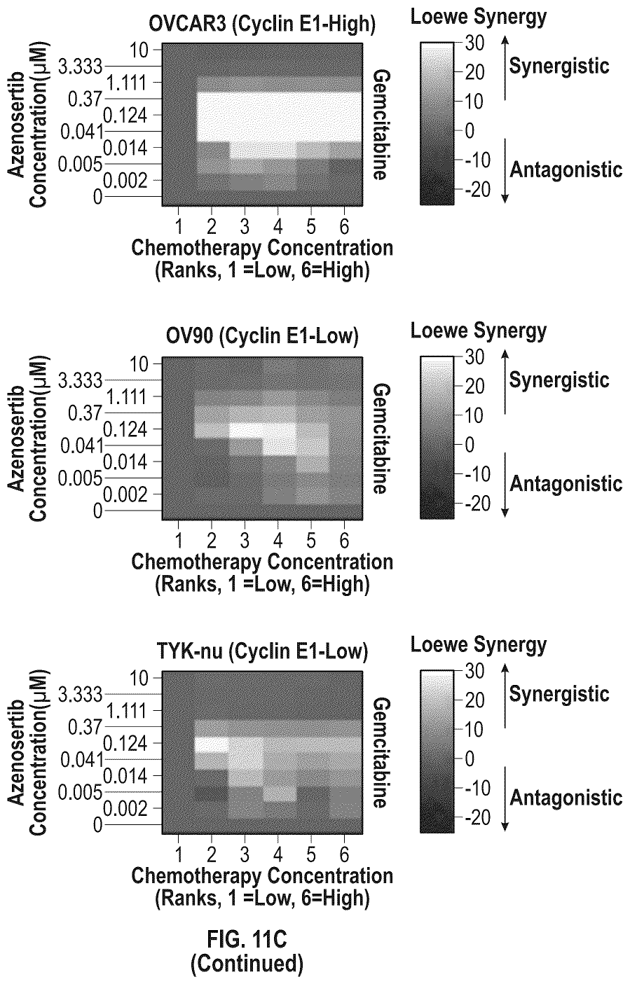

To evaluate the effects of Azenosertib in combination with paclitaxel, mice were inoculated with A 2780 (non-CCNE1-amplified, CN=2) (FIGS. 10A and 10B) or OVCAR3 (CCNE1 amplified, CN=14) (FIG. 11A and FIG. 11B) cells. Animals were randomized and treated as summarized in Table 2 once the mean tumor volume reached a specified size. M ice treated with Azenosertib and paclitaxel demonstrated reduction in tumor growth compared to vehicle treated control animals. Tumor regression was observed in animals with CCNE1 amplified tumors as shown in FIGS. 11A and 11B. The Cyclin E1-high OVCAR3 model (46% reduction of initial tumor volume/104% TGI) and the Cyclin E1-low A 2780 model (85% TGI). (Table 2). These results demonstrate that CCNE1-amplified tumors are more sensitive to combination therapy (e.g., Azenosertib and paclitaxel combination) compared to non-CCNE1-amplified tumors.

TABLE 2
Dosing Schedule for Azenosertib and Paclitaxel Combination Studies

| | | | |
|---|---|---|---|
| Treatment | Azenosertib | Paclitaxel | Tumor Growth |
| Group | Dose | Dose | Inhibition |

A2780 non-CCNE1-amplified (CN = 2) (Cyclin E1-low)

| | | | |
|---|---|---|---|
| Vehicle Control | — | — | — |
| 1 | 60 mg/kg QD × 14 | — | 34% |
| 2 | — | 10 mg/kg qw × 1 | 46% |
| | | (PG-D 0-D 6) | |
| | | biw × 1 | |
| | | (PG D 7-D 13) | |
| 3 | 60 mg/kg QD × 18 | 10 mg/kg qw × 1 | 85% |
| | | (PG-D 0-D 6) | |
| | | biw × 1.5 | |
| | | (PG D 7-D 17) | |

OVCAR3 CCNE1-amplified (CN = 14) (Cyclin E1-high)

| | | | |
|---|---|---|---|
| Vehicle Control | — | — | — |
| 1 | 60 mg/kg QD × 28 | — | 68% |
| 2 | — | 5 mg/kg 4 days on, | 74% |
| | | 3 days off × | |
| | | 4 cycles | |
| 3 | 60 mg/kg QD × 28 | 5 mg/kg 4 days on, | 104% |
| | | 3 days off × | Tumor |
| | | 4 cycles | Regression |

Cyclin E1-high OVCAR3 cells showed greater synergistic effects (Loewe synergy score >10 is synergistic, <−10 is antagonistic) in all chemotherapy and Azenosertib combinations than Cyclin E1-low OV90 and TYK-nu cells (FIG. 11C). The drug combination effect was evaluated by measurement of cell viability and 4 calculation methods (ZIP, Bliss, Loewe and HSA) following a SynergyFinder guidelines. The Loewe score was consistent with other methods of calculating synergy. Scores were capped at 30 for visualization purposes. Each panel represents the summary of 3 to 6 replicates. The concentration of chemotherapy was rank-transformed and its ranges were cell line and chemotherapy specific: oxaliplatin: 0-10 μM (OVCAR3, OV90), 0-3.3 UM (TYK-nu); paclitaxel: 0-0.005 UM (OVCAR3), 0-0.02 UM (OV90, TYK-nu); gemcitabine: 0-1 μM (OVCAR3), 0-0.02 μM (OV90), 0-0.01 μM (TYK-nu).

### Example 6. Clinical Trial to Treat Cancer in Subjects with CCNE1 Gene Amplification

The effectiveness of Azenosertib in combination with a second chemotherapeutic agent in human subjects having CCNE1 gene amplification was assessed through clinical trials. Selected subjects have high-grade serous ovarian cancer (HGSOC), platinum-resistant or refractory, 1 or 2 prior chemotherapies and measurable disease. Subjects were administered Azenosertib in combination with PLD, carboplatin, paclitaxel, and gemcitabine.

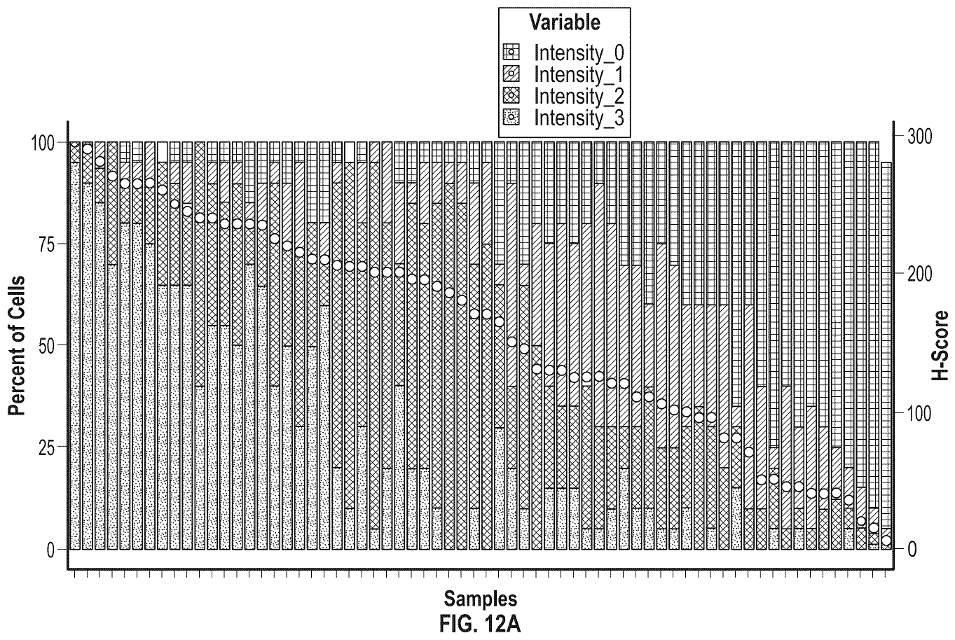

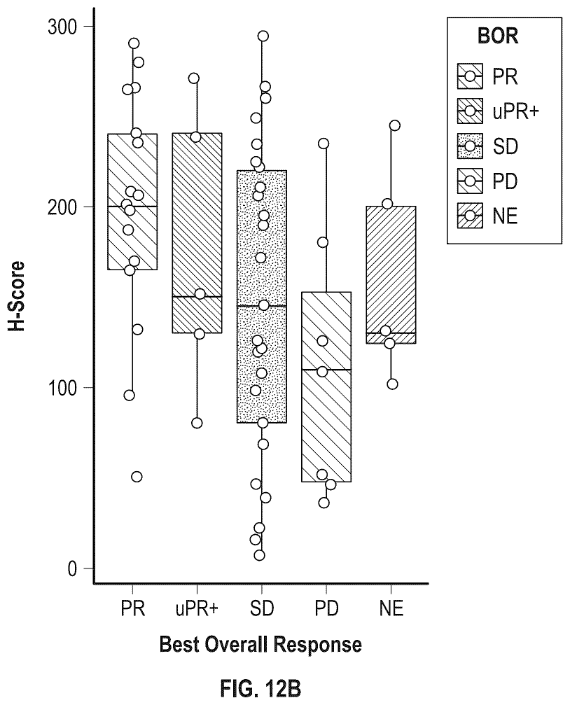

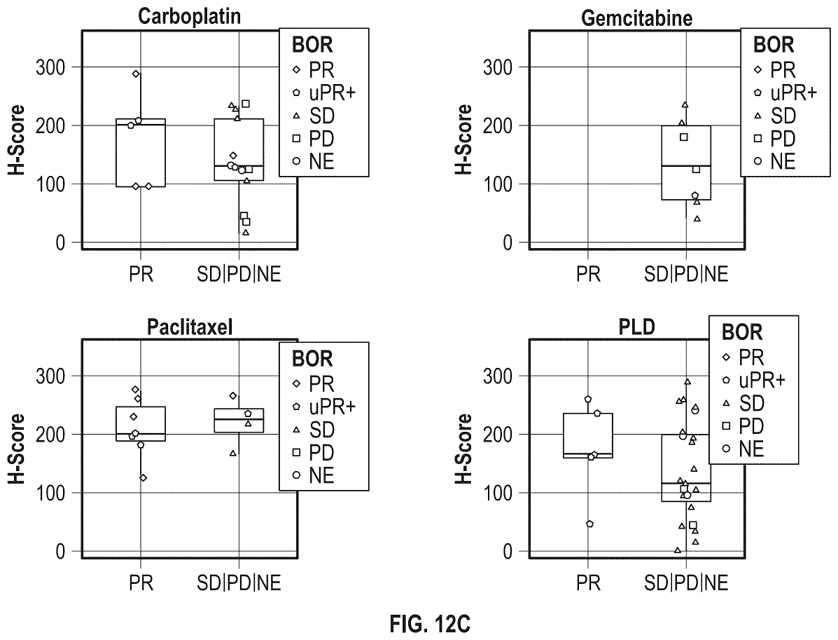

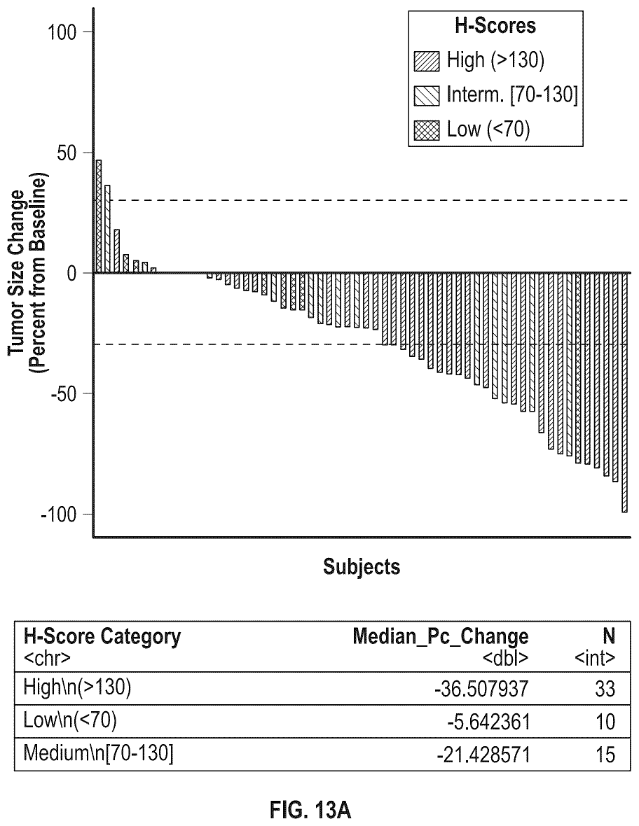

Cyclin E1 expression levels were determined using immunohistochemistry (IHC) and summarized as an H-score for each tumor specimen from 63 subjects enrolled in the clinical study. Of the 63 subjects included in IHC testing, a total of 58 were evaluated for best overall response (BOR), including Partial Response or PR (N=17), Stable Disease or SD (N=34), Progressive Disease or PD (N=7), and Not Evaluable NE (N=5). These included subjects from all four arms of the study: carboplatin (N=18), paclitaxel (N=11), PLD (N=28), and gemcitabine (N=6). Each IHC microscopy image was reviewed by a board-certified pathologist and used to evaluate the Cyclin E1 expression level. The epithelial cells from the tumor area were classified by Cyclin E1 staining intensity (SI): absent (SI=0), low (SI=1+), medium (SI=2+) and high (SI=3+). (FIG. 12A) A summary H-score was calculated from the proportion of cells in each class weighted by their SI as

$$H - Score = f 1 + 2 ⋆ f 2 + 3 ⋆ f 3$$

with fi the fraction of cells with SI=i (where i=1+, 2+, or 3+)

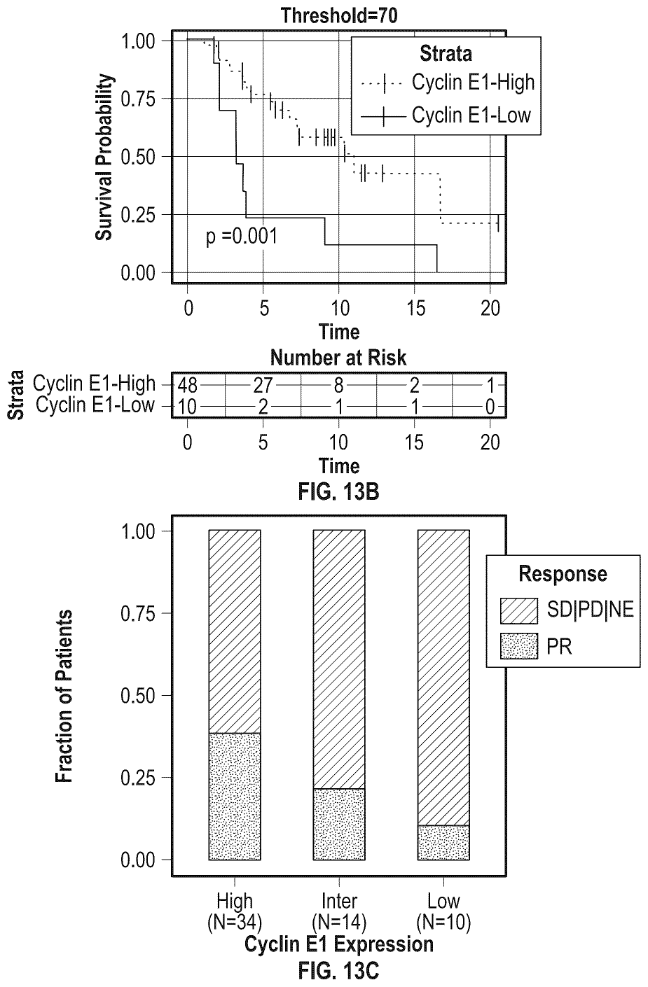

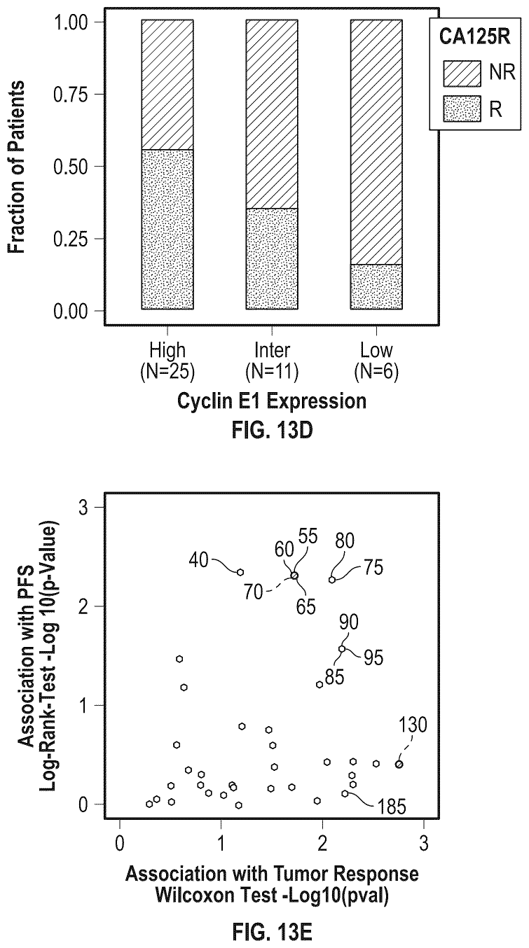

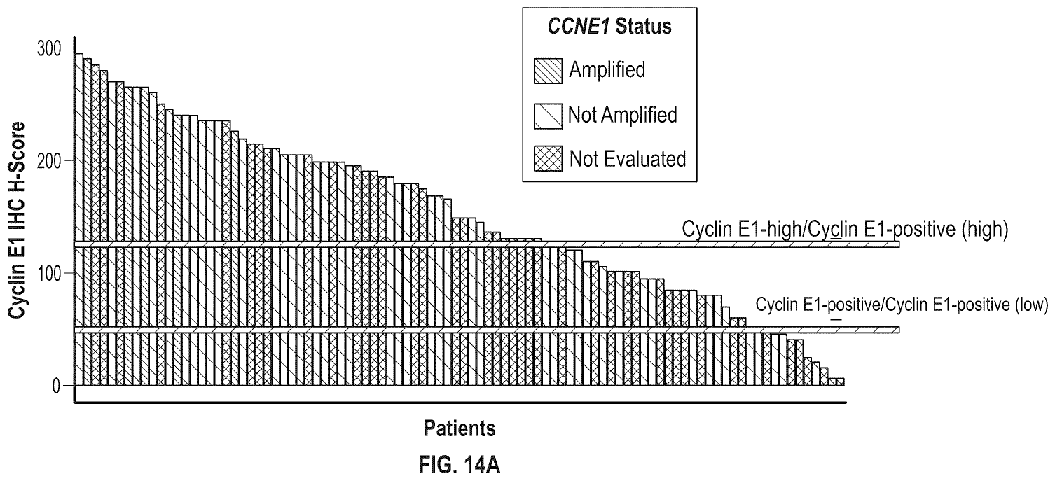

Best Overall Response was evaluated using (PR) vs (PD+SD+NE+uPR+) criteria. Subjects with Cyclin E1 H-score greater than 130 were shown to be more likely to respond (ORR=47% vs 16%, p=0.01 Fisher exact test) and Cyclin E1 H-scores were higher in PR subjects compared to PD subjects (FIGS. 12B and 12C). Subjects were evaluated based on change in tumor size from baseline. As shown in FIG. 13A, subjects with intermediate (70-130) and high (>130) Cyclin E1 H-scores demonstrated superior tumor response compared to subjects with low Cyclin E1 H-scores (<70). The subjects with highest expression (H-score >130) have larger tumor responses (−34% vs −12%, p=0.001 Wilcoxon test). Low expression subjects (H<70, N=10) had significantly shorter progression free survival (PFS) (3.25 vs 10.35 months, p=0.0027 log rank test (FIG. 13B). The fraction of overall responders (FIG. 13C) or CA 125 responders (FIG. 13D) decreases with increasing H-scores. Groups were defined based on optimal association with tumor response (High Group) and PFS (Low Group) using H-score of 130 as a threshold associated with tumor response and H-score of 70 associated with PFS. (FIG. 13E).

### Example 7. Clinical Trial to Treat Cancer in Subjects with CCNE1 Gene Amplification

This example describes a clinical trial carried out to assess safety and efficacy of treatment with Azenosertib in combination with a second exemplary chemotherapeutic agent in subjects with metastatic high-grade epithelial ovarian, peritoneal, or fallopian tube cancer (EOC) after two or fewer lines of chemotherapy, including, in some embodiments, platinum chemotherapy.

Azenosertib was administered continuously or intermittently once a day in 21- or 28-day cycles together with a second chemotherapeutic agent. In some embodiments, the second chemotherapeutic agent is selected from pegylated liposomal doxorubicin, carboplatin, paclitaxel, and gemcitabine. The study was designed to assess safety and establish maximum tolerated dose of each combination, besides clinical activity. In one embodiment, Azenosertib (Azenosertib) was tested in combination with paclitaxel. Azenosertib was administered orally once daily in 28-day treatment cycles in two doses in an intermittent dosing regimen starting at 200 mg QD 5 days on/2 days off (5/2) and subsequently 300 mg QD 5/2. Paclitaxel was administered intravenously over 60 minutes (+10 minutes) at a dose of 80 mg/m² on D1, D8 and D15 of each 28-day cycle.

In one embodiment, Azenosertib (Azenosertib) was tested in combination with carboplatin. Azenosertib was administered orally once daily in 28-day treatment cycles in four doses in an intermittent dosing regimen starting at two doses of 300 mg QD 5/2 and subsequently two doses at 200 mg QD 5/2. Carboplatin was administered intravenously at 5 mg/mL*min over 15 minutes or longer, on Day 1 of each 21-day cycle (+3 days).

In one embodiment, Azenosertib (Azenosertib) was tested in combination with gemcitabine. Azenosertib was administered orally once daily in 28-day treatment cycles in four doses in an intermittent dosing regimen starting at three doses of 200 mg QD and subsequently 200 mg QD 5/2. Gemcitabine was administered intravenously at two doses of 1000 mg/m² and 600 mg/m² intravenously over 30 minutes or longer, on Day 1 and Day 8 of each 21-day cycle.

In one embodiment, Azenosertib (Azenosertib) was tested in combination with pegylated liposomal doxorubicin (PLD). Azenosertib was administered orally once daily in 28-day treatment cycles in three doses in an intermittent dosing regimen starting at 200 mg QD followed by 400 mg QD 5/2. PLD was administered at a dose of 40 mg/m² intravenously over 60 minutes every 4 weeks, on Day 1 of each 28-day cycle.

The endpoints were to determine a Recommended Phase 2 Dose (RP2D), safety, and preliminary clinical activity. From these clinical studies, the RP2D was determined to be: (a) Azenosertib 300 mg QD 5/2 in combination with paclitaxel 80 mg/m² on D1, D8, D15 (28-day cycles); (b) Azenosertib 200 mg QD 5/2 in combination with carboplatin AUC5 mg/mL*min on D1 (21-day cycles); (c) Azenosertib 400 mg QD 5/2 in combination with PLD 40 mg/m² D1 (28-day cycles). Azenosertib in combination with gemcitabine has durable activity and dose cohorts are ongoing for determination of maximum tolerated dose (MTD).

The Overall Response Rate and Median Progression Free Survival from the study is shown in Table 3 below. Overall response rate (ORR) refers to the proportion of subjects in the trial whose tumor is significantly reduced or destroyed upon treatment. Median Progression Free Survival (mPFS) refers to the length of time from either the date of diagnosis or the start of treatment such that half of the subjects diagnosed with the disease or tumor are still alive. It provides an indication of the success of a treatment.

TABLE 3
Combination therapy with Azenosertib and chemotherapeutic agent

| | Azenosertib + | | | | |
|---|---|---|---|---|---|
| | pegylated | | | | |
| | liposomal | Azenosertib + | Azenosertib + | Azenosertib + | |
| | doxorubicin | carboplatin | paclitaxel | gemcitabine | Total |
| | (n = 35) | (n = 27) | (n = 18) | (n = 14) | (N = 94) |
| Overall Response Rate | 14.3 | 33.3 | 50.0 | 14.3 | 26.6 |
| (ORR) % | | | | | |
| Subjects with | 29 | 23 | 15 | 13 | 80 |
| immunohistochemistry | | | | | |
| (IHC) available (N) | | | | | |
| Overall Response Rate | | | | | |
| by Cyclin E1 (n/N; %) | | | | | |
| High | 4/23; 17.4 | 7/18; 38.9 | 8/15; 53.3 | 2/11; 18.2 | 21/67; 31.3 |
| Low | 1/6; 16.7 | 0/5; 0 | 0/0; 0 | 0/2; 0 | 1/13; 7.7 |
| Median Progression | 9.03 | 4.24 | 7.36 | NE | 9.03 |
| Free Survival (PFS), | | | | | |
| months | | | | | |
| Median Progression | | | | | |
| Free Survival (PFS) by | | | | | |
| Cyclin E1, months | | | | | |
| High | 11.01 | 10.35 | 7.36 | NE | 10.35 |
| Low | 3.75 | 2.14 | NE | NE | 3.25 |
| Hazard Ratio (HR) | 0.4 | 0.1 | NE | NE | 0.03 |

Of the 103 subjects enrolled, 26.6% had a partial response and median progression free survival of 9.03 months (95% CI: 5.52-11.01). The results showed that Azenosertib and paclitaxel demonstrated the highest Overall Response Rate (ORR) (9/18 (50%)), followed by carboplatin (9/27 (33.3%)).

Overall Response Rate for Azenosertib when administered in combination with pegylated liposomal doxorubicin or when administered in combination with gemcitabine was 14.3% (5/35, 2/14 respectively).

Of 80 subjects for which Cyclin E1 expression data by immunohistochemistry (IHC) was evaluated, higher Cyclin E1 (using a threshold of H-score >50) correlated with higher Overall Response Rate (ORR=31.3% vs 7.7%) and longer progression free survival (PFS=10.35 vs 3.25 months, HR=0.3). A hazard ratio (HR) of 0.5, for example, means that half as many subjects in the active group have an adverse event at any point in time compared with placebo. Frequent grade ≥3 treatment emergent adverse events (TEAEs) (%) observed were neutropenia (44.4), thrombocytopenia (30.3), anemia (12.1), leukopenia (11.1), fatigue (10.1), diarrhea (6.1), nausea (5.1), and vomiting (5.1).

At another timepoint, 115 subjects were enrolled in the study and 94 were efficacy evaluable with a median progression free survival (mPFS)=9.0 months (95% CI: 5.8-13.7). Azenosertib in combination with Paclitaxel demonstrated the highest confirmed ORR of 50% (mPFS of 7.4 m), followed by Gemcitabine 38.5% (mPFS of 10.4 m), Carboplatin 35.7% (mPFS of 8.3 m), and PLD 19.4% (mPFS of 6.3). A total of 82 response-evaluable subjects had available Cyclin E1 expression data by IHC. Cyclin E1-positive status (H-score >50) correlated with higher ORR and longer PFS (ORR=40.0% vs 8.3%; PFS=9.86 vs 3.25 months, HR=0.37; P-value of 0.0078). Frequent Grade ≥3 related TEAEs (%) in the intermittent Azenosertib treatment groups were thrombocytopenia (12.2), neutropenia (11.3), anemia (7.0), fatigue (4.3), nausea (1.7), vomiting (1.7), and diarrhea (0.9).

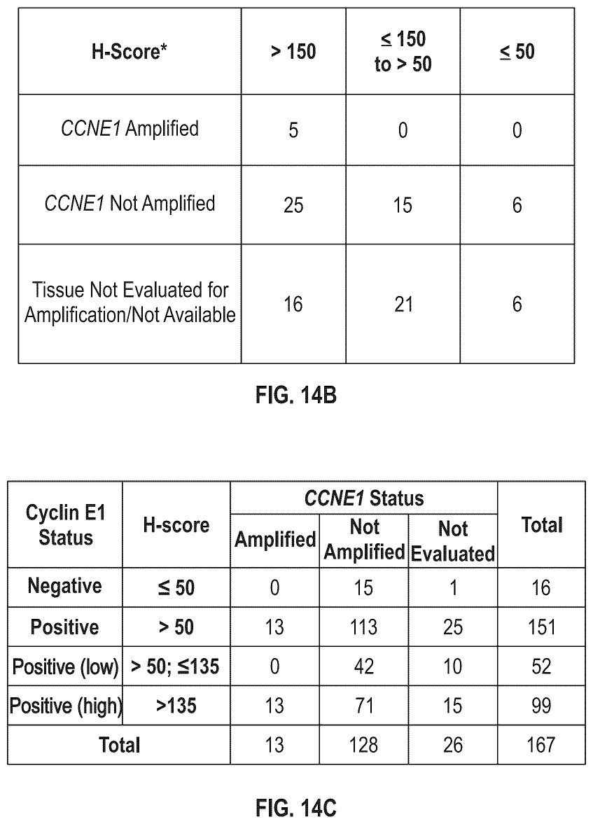

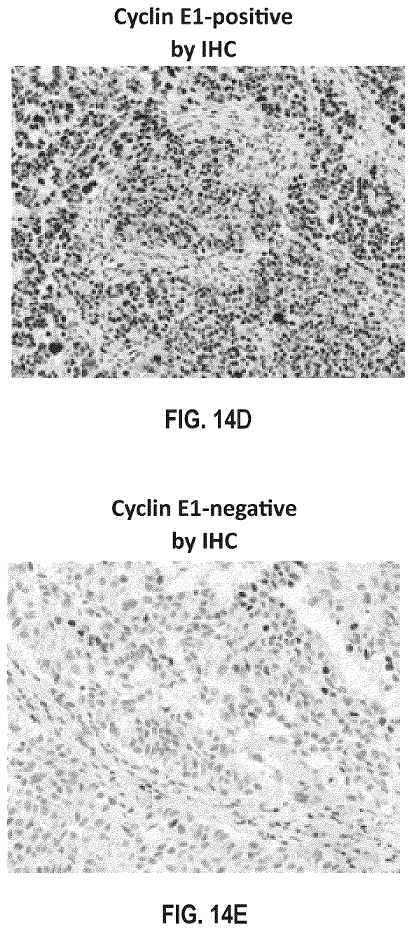

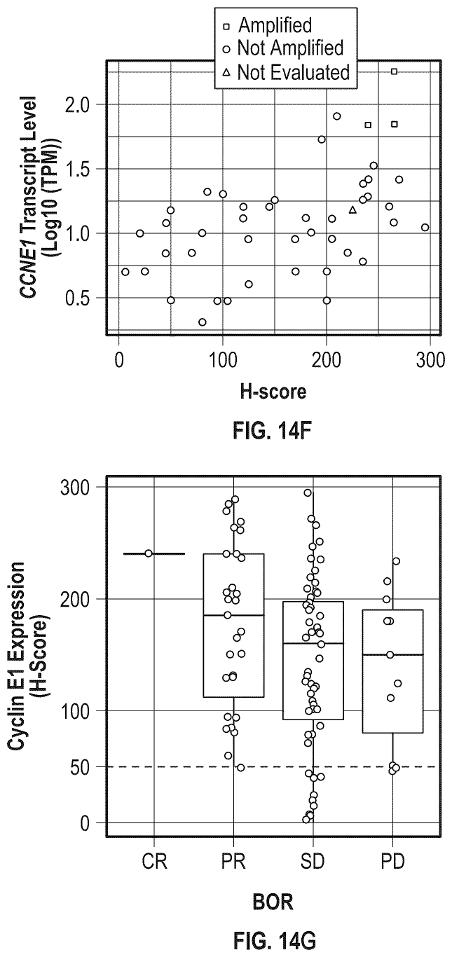

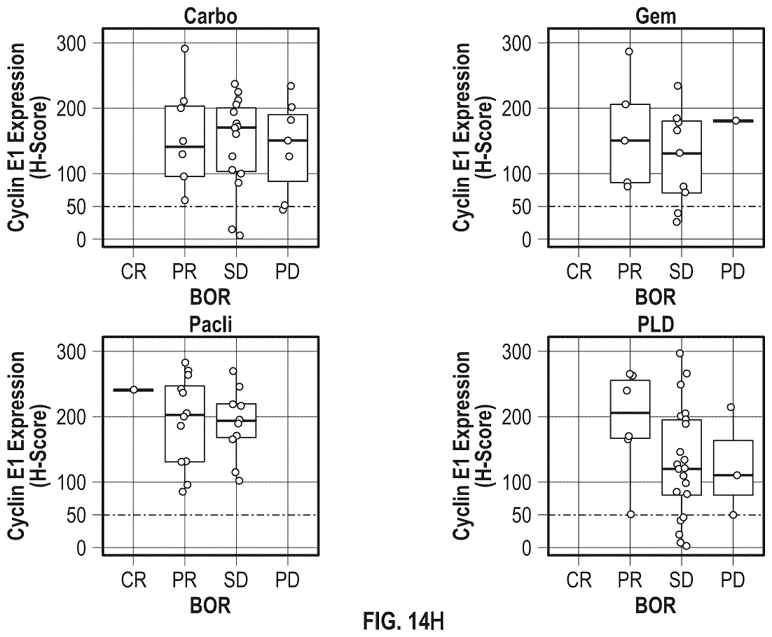

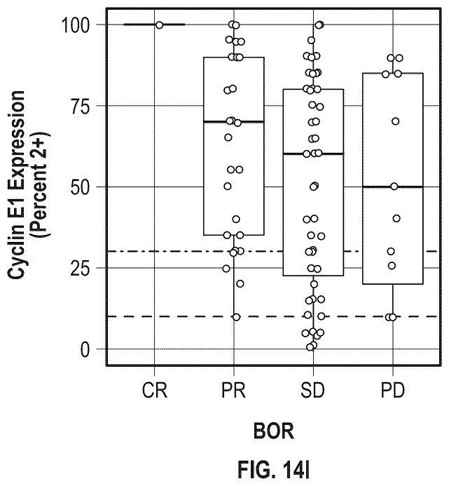

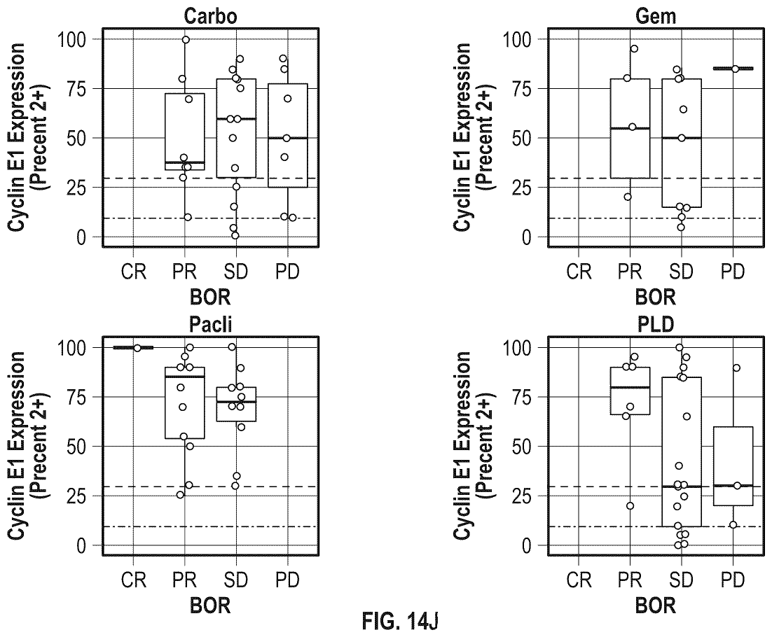

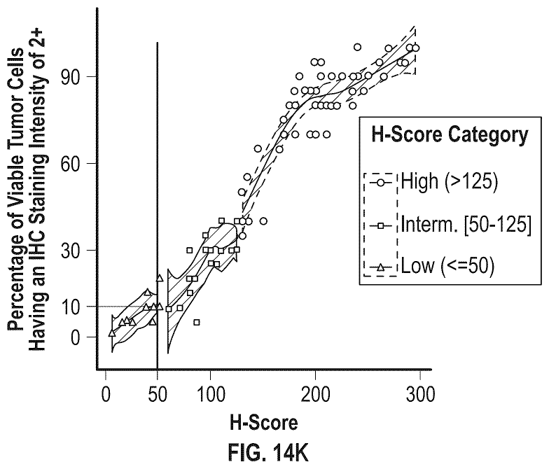

Subject samples were also evaluated for CCNE1 gene amplification. As shown in FIGS. 14A-14C, an H-score >50 includes all CCNE1-amplified tumors. H-scores were calculated by multiplying the percentage of cells (0 to 100%) with intensity of Cyclin E1 expression (0, 1, 2+, 3+).

Subjects were classified as Cyclin E1-positive (H>50) or Cyclin E1-high (H>135, the lowest score observed in samples with CCNE1 gene amplification). 90% (151/167) were Cyclin E1-positive, 59% (99/167) were Cyclin E1-high, 9% (13/141) of evaluable samples were CCNE1-amplified, and 85% (71/84) of Cyclin E1-high evaluable samples were not CCNE1-amplified. Cyclin E1-positive expression is highly prevalent including in subjects with no CCNE1 gene amplification. CCNE1 transcript levels were evaluated in subject samples and highly correlated with Cyclin E1 H-score (rho=0.5, p<0.001) (FIG. 14D).

Cyclin E1 status, including CCNE1 gene amplification predicts the benefit of Azenosertib in addition to chemotherapy, suggesting that Azenosertib restores chemotherapy sensitivity in heavily pre-treated platinum resistant ovarian cancer.

These results showed that administration of Azenosertib in combination with chemotherapeutic agents was well tolerated and has clinical activity, with durable responses in subjects with platinum resistant or refractory metastatic high-grade epithelial ovarian, peritoneal, or fallopian tube cancer. Subjects with Cyclin E1 overexpressing tumors (Cyclin E1-positive), a subgroup known for suboptimal benefits from chemotherapy, demonstrated significant improvements in ORR and PFS, compared to subjects with tumors having low Cyclin E1 expression (Cyclin E1-negative). FIG. 14E is an exemplary image of tumor cells being designated with a Cyclin E1-positive status while FIG. 14F is an exemplary image of tumor cells being designated with a Cyclin E1-negative status.

Azenosertib in combination with chemotherapy demonstrated strong anti-tumor activity in a heavily pretreated population, with an ORR of 50% in combination with paclitaxel, 35.7% with carboplatin, and 38.5% with gemcitabine. Azenosertib confers higher objective response rates than historical chemotherapy alone rates or chemotherapy combined with other WEE1 inhibitors. Increases in responses for the Azenosertib combinations independent of the type of chemotherapy utilized. Subjects with Cyclin E1-positive tumors (e.g., H-score greater than 50) benefited in chemotherapy combination arms illustrating synergy between Azenosertib and chemotherapy in this subject population. As shown in FIG. 14G to FIG. 14J, subjects with an IHC H-score greater than 50 or a percentage of viable tumor cells having a Cyclin E1 IHC staining intensity of 2+ of above 10% achieved partial responses following combination therapy with Azenosertib. FIG. 14K is a graph mapping a percentage of viable tumor cells having a staining intensity of 2+ to an H-score cut-offs for Cyclin E1 immunohistochemistry (IHC), showing that an H-score of >50 subject population is correlated with >10% of viable tumor cells having a staining intensity of 2+ and an H-score of >125 subject population is correlated with >30% of viable tumor cells having a staining intensity of 2+.

Azenosertib was well-tolerated in combination with multiple types of chemotherapy and demonstrated encouraging clinical activity, including durable objective responses in subjects with platinum-resistant ovarian cancer. The addition of Azenosertib increased objective response rates (ORRs) and median progression free survival (mPFS) over those observed historically with chemotherapy alone, or with chemotherapy in combination with adavosertib. Particularly promising are the improvements in ORR and mPFS observed in subjects with Cyclin E1-positive tumors, a subgroup recognized to have a poor prognosis and be refractory to chemotherapy. Moreover, the tolerability and durable efficacy of Azenosertib in combination with paclitaxel or carboplatin compares favorably to historical data from chemotherapy doublets of either paclitaxel-carboplatin or PLD-carboplatin.

### Example 8. Cyclin E1-Positive Status is Independent of Prior Platinum Treatment in High Grade Serous Ovarian Cancer

HGSOC tumor samples from 167 subjects were obtained from an ongoing Azenosertib clinical trial (N=111) (NCT04516447) evaluating Azenosertib in Combination with Chemotherapy in Subjects With Platinum-Resistant Ovarian, Peritoneal or Fallopian Tube Cancer. Cyclin E1 protein expression levels were measured through immunohistochemistry assay using anti-Cyclin E1 mouse monoclonal antibody (Abcam Cyclin E1/2460). H-score was defined as the weighed sum of the percent (pc) or fraction of cells stained at increasing intensity (1*pc1+2*pc2+3*pc3). Subjects were classified as Cyclin E1-positive (H>50, the previously reported predictive threshold in this study) or Cyclin E1-high (H>135, the lowest score observed in samples with CCNE1 gene amplification).

CCNE1 gene copy number (N=141) and Homologous Recombination and Repair (HRR) genes mutational status (N=86) was obtained from tissue-based genomic profiling. The CCNE1 gene amplification status (amplified, not amplified or non-amplified) was obtained from clinical assays or using a minimum of 6 copies to call gene amplification (research assays). Transcript abundance (N=49) was obtained using central Caris MI Profile assay and measured as Transcripts per Million Reads (TPM). Clinical and pathological variables, including treatment modalities and outcomes, were obtained primarily from the clinical data in this study and supplemented by data associated with procured specimen when available (age, collection method, etc.). There was no statistically significant association between histological variables (anatomical location, site of collection, collection method, tumor cellularity, format received or tissue age at IHC assay) examined and Cyclin E1 IHC H-score (data not shown). The distribution of Cyclin E1 expression measured by IHC was independent of pre-analytical variables.

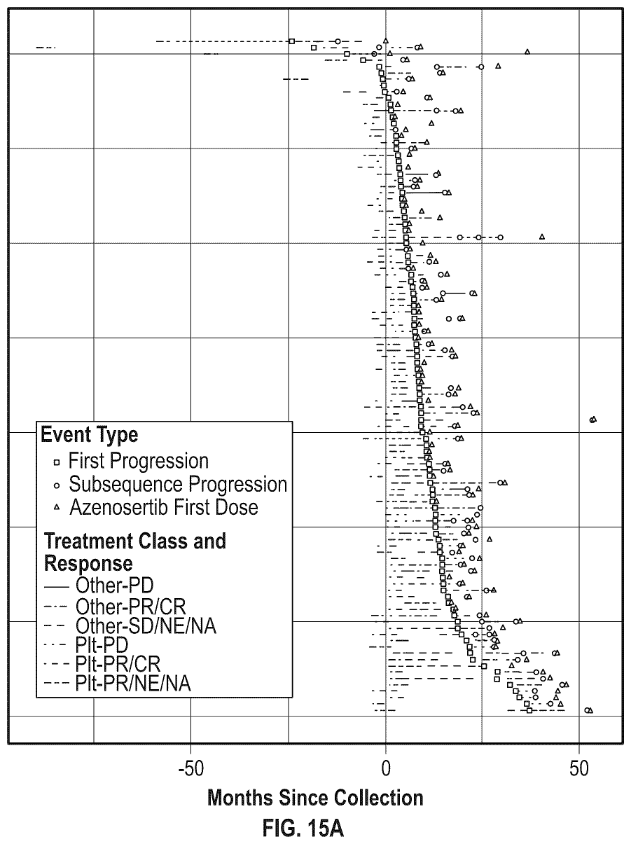

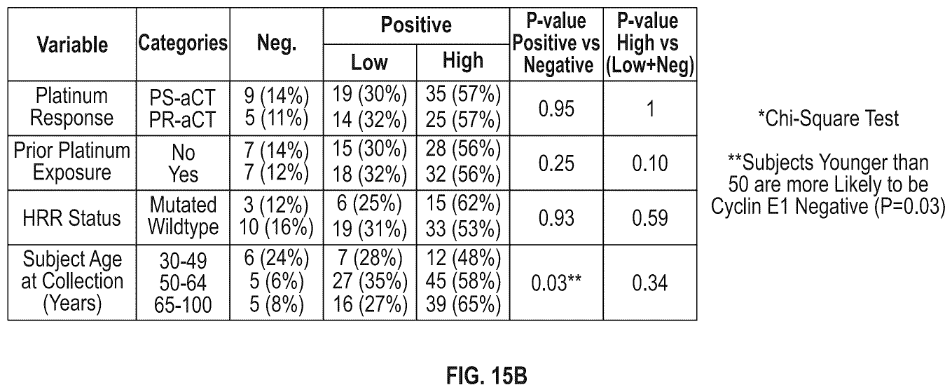

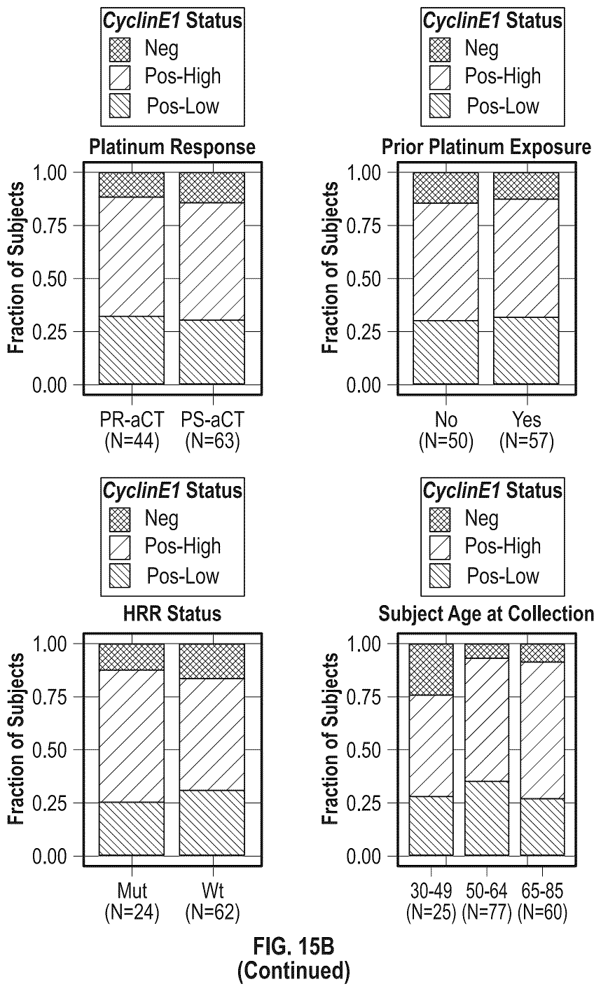

Platinum response classification was established from analysis of prior systemic therapy time courses and responses (FIG. 15A). Subjects whose specimen collection met one of the following criteria were classified as Platinum Sensitive at Collection Time (PS-aCT, N=63/107):
Specimen collected prior to any platinum exposure
Specimen collected after the first platinum exposure, but prior to subsequent platinum treatment in subjects and with at least 6 months Platinum Free Interval (PFI) after the last dose
Specimen collected during the first or second platinum treatment in subjects with at least 6 months PFI after the last dose
Specimen collected at intervening surgery between neoadjuvant and adjuvant platinum treatment in subjects with at least 6 months PFI after last dose

All others evaluable cases were classified as Platinum Resistant/Refractory at Collection Time (PR-aCT, N=44/107). As shown in FIG. 15B, platinum exposure, response or HRR mutational status did not significantly impact the Cyclin E1 expression-based classification of the subjects.

### Example 9. Treating Cancer in Subjects Selected to have Cyclin E1 Biomarker Levels Above a Predetermined Threshold

As discussed above, CCNE1 gene amplification and/or Cyclin E1 expression functions as a marker for the enrichment of subject populations for treatment with Azenosertib. These data demonstrate that Azenosertib drives cancer cell death in Cyclin E1-high tumor cells in vitro and substantially inhibits the growth of Cyclin E1-high subject-derived in vivo tumor models. Additionally, these data support the use of CCNE1 gene copy number and/or Cyclin E1 protein expression as predictive markers to significantly improve subject outcomes by enabling selection of optimal subjects for treatment with Azenosertib.

This example demonstrates treating subjects selected to have a predetermined Cyclin E1 status or Cyclin E1 biomarker level above a predetermined threshold by administering an effective dose of Azenosertib (e.g., 175 mg, 200 mg, 225 mg, 250 mg, 275 mg, 300 mg, 325 mg, 350 mg, 375 mg, 400 mg, 450 mg, or 600 mg) alone or in combination with a second chemotherapeutic agent. Subjects are selected based on whether the subjects tumor tissue has (1) CCNE1 gene amplification (e.g., copy number at or greater than 5); (2) Cyclin E1 overexpression (e.g., mRNA or IHC H-score greater than 50 or a percentage of viable tumor cells having a Cyclin E1 IHC staining intensity of 2+ of above 10%). Additional selection criteria may include subjects that have a specific cancer type (e.g., high-grade serous ovarian cancer (HGSOC)), platinum-resistant or refractory, or 1-3 prior lines of therapy (e.g., bevacizumab).

Subjects are also selected based on having High-Grade Serous Ovarian Cancer; ECOG PS 0-1; Platinum-resistant (excluding Platinum refractory); 1-3 prior lines of chemotherapy; Measurable disease per RECIST v 1.1; Cyclin E1-positive (e.g., IHC+) and/or CCNE1-amplified.

This Phase 3 study compares Azenosertib in combination with either carboplatin or paclitaxel with traditional doublet chemotherapy in platinum-sensitive ovarian cancer. The Phase 3 trial focused on Cyclin E1-positive ovarian cancer, supported by positive Phase 1b clinical data. Subjects with Cyclin E1-positive tumors have been shown to be refractory to chemotherapy alone and generally have poor prognosis.

Subjects are administered Azenosertib at a dose of 400 mg QD for 5 days followed by 2 days without Azenosertib dosing a week (5:2). Subjects are evaluated for ORR following treatment and divided into the following treatment groups:
Cohort 1 (N=30) subjects have confirmed Cyclin E1-positive (e.g., IHC+) and/or CCNE1-amplified
Cohort 2A (N=60) subjects have confirmed CCNE1 amplification.
Cohort 2B (N=80) subjects have non-CCNE1-amplified tumors and are Cyclin E1-positive (IHC+).
Cohort 2C (N=40) subjects have non-CCNE1-amplified tumors and are Cyclin E1-low (e.g., IHC-low or IHC-negative (IHC−)).

Additional subject groups are administered Azenosertib and chemotherapy in Recurrent Platinum-Sensitive Ovarian Cancer that is Cyclin E1-positive. Eligibility criteria include subjects having confirmed High-Grade Serous Ovarian Cancer; ECOG performance status 0-1; ≥1 L Prior Line of Platinum-based chemotherapy; Platinum Sensitive (Platinum-free interval ≥6 months); Prior Bevacizumab & PARPi if eligible and per regional standard of care; Cyclin E1-positive (either CCNE1-amplified and/or Cyclin E1 IHC+).

Subjects receiving the combination therapy are stratified based on stratification factors including Prior lines of therapy (1 v 2-3); Prior PARPi (Y es v No); and CCNE1 amplification (Y es v No). Subjects are randomized into two groups. Group 1 receives Azenosertib+chemotherapy (paclitaxel or carboplatin) for 6 cycles followed by Azenosertib maintenance receiving 400 mg QD 5:2. Group 2 receives Azenosertib+carboplatin doublet (Paclitaxel or Pegylated Liposomal Doxorubicin) for 6 cycles without a subsequent Azenosertib maintenance period. Exemplary dosing for combination therapy groups are shown below in Table 4.

TABLE 4
Exemplary Combination Therapy Doses

| | Azenosertib | Chemotherapy |
|---|---|---|
| Paclitaxel | 300 mg QD 5:2 | 80 mg/m² on D 1, |
| | | D 8, D 15 (28-day cycles) |
| Carboplatin | 200 mg QD 5:2 | AUC = 5 on D 1 |
| | | (21-day cycles) |
| PLD | 400 mg QD 5:2 | 40 mg/m² D 1 |
| | | (28-day cycles) |

Subjects are assessed for primary and secondary endpoints including progression-free survival determined by a blinded independent central review and overall survival.

### Example 10. Azenosertib Demonstrates Cancer Response in Heavily Pre-Treated Subjects

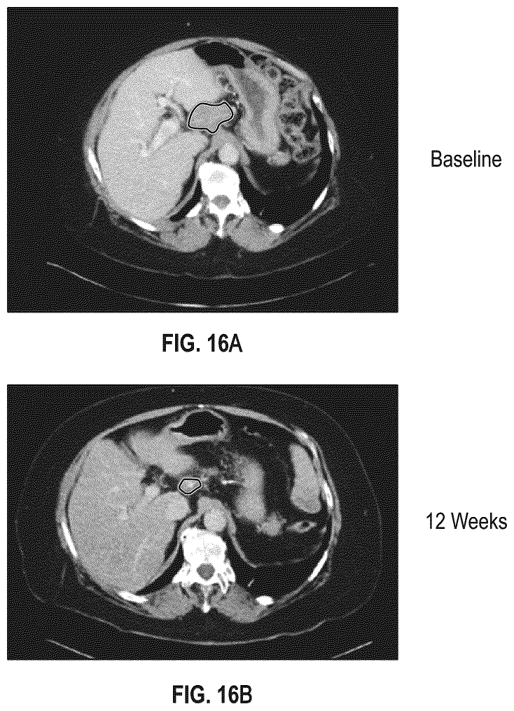

Azenosertib has demonstrated efficacy in an exemplary human subject with CCNE1-amplified platinum-resistant ovarian cancer. The subject was selected to have CCNE1-amplified status (confirmed by Foundation assay) and was administered Azenosertib in accordance with an intermittent dosing schedule of at least 400 mg Azenosertib once a day for five consecutive days, followed by two off days for 11 months. The subject is a 73-year-old female who received 10 prior lines of therapy: (1) Avelumab (SD); (2) Doxorubicin Liposomal (PD); (3) Topotecan/bevacizumab (PD); (4) Cyclophosphamide/bevacizumab (unk); (5) XMT1536 (NaPi2b ADC) (PR); (6) APG115 (MDM2 inh)/Pembrolizumab (SD); (7) ABBV-155 (CD275 ADC) (PD); (8) NC 318 (Siglec-15 mA B) (SD); (9) SM 08502 (CLK inhibitor) (PD); (10) NBMBMX (HDAC8 inh) (SD). Following treatment with Azenosertib, the subject demonstrated durable cPR of −71% and visible reduction in the target lesion (FIGS. 16A and 16B). This exemplary subject demonstrates further support for treating platinum-resistant cancers using Azenosertib and CCNE1 gene amplification status as a biomarker.

### EQUIVALENTS AND SCOPE

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure. The scope of the present disclosure is not intended to be limited to the above Description, but rather is as set forth in the following claims.

1. A method of treating cancer comprising:
administering to a subject selected to have a predetermined Cyclin E1 status, an effective dose of Azenosertib, or a pharmaceutically acceptable salt thereof.

2. A method of treating cancer comprising:
administering to a subject selected to have a Cyclin E1 biomarker level above a predetermined threshold, an effective dose of Azenosertib, or a pharmaceutically acceptable salt thereof.

3. A method of treating cancer comprising:
administering to a subject selected to have a Cyclin E1 biomarker level above a predetermined threshold, an effective dose of Azenosertib, or a pharmaceutically acceptable salt thereof, and a second chemotherapeutic agent, or a pharmaceutically acceptable salt thereof.

4. The method of claim 1, wherein the predetermined Cyclin E1 status is Cyclin E1-positive, Cyclin E1-positive (low), Cyclin E1-positive (high), or Cyclin E1-high.

5. The method of claim 4, wherein the predetermined Cyclin E1 status is Cyclin E1-positive.

6. The method of claim 4, wherein the predetermined Cyclin E1 status is Cyclin E1-high.

7. The method of any one of claims 1 to 6, wherein the Cyclin E1 status or the Cyclin E1 biomarker level is measured by a Cyclin E1 protein expression level.

8. The method of claim 7, wherein the Cyclin E1 protein expression level is determined by CCNE1 mRNA or transcript levels.

9. The method of claim 7, wherein the Cyclin E1 protein expression level is determined by protein levels.

10. The method of any one of claims 1 and 5 to 9, wherein the predetermined Cyclin E1 status is a Cyclin E1 protein expression level above a predetermined cut-off.

11. The method of any one of claims 1 and 5 to 10, wherein the predetermined Cyclin E1 status is an immunohistochemistry (IHC) status.

12. The method of any one of claims 2, 4, and 8 to 12, wherein the predetermined cut-off or the predetermined threshold is measured by a percentage of viable tumor cells having a Cyclin E1 immunohistochemistry (IHC) staining intensity of 2+.

13. The method of claim 12, wherein the predetermined cut-off or the predetermined threshold is a percentage of viable tumor cells having a Cyclin E1 IHC staining intensity of 2+ of IHC above 7%, 8%, 9%, 10%, 11%, 12%, 13%, 14%, 15%, 16%, 17%, 18%, 19%, 20%, 21%, 22%, 23%, 24%, 25%, 26%, 27%, 28%, 28%, 29%, 30%, 31%, 32%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, or 62%.

14. The method of claim 13, wherein the predetermined cut-off or the predetermined threshold is a percentage of viable tumor cells having a Cyclin E1 IHC staining intensity of 2+ of IHC above 10%.

15. The method of claim 13, wherein the predetermined cut-off or the predetermined threshold is a percentage of viable tumor cells having a Cyclin E1 IHC staining intensity of 2+ of IHC above 30%.

16. The method of any one of claims 2, 4, and 10 to 15, wherein the predetermined cut-off or the predetermined threshold is measured by a Cyclin E1 immunohistochemistry (IHC) H-score.

17. The method of claim 16, wherein the predetermined cut-off or the predetermined threshold is a Cyclin E1 IHC H-score of above 45, 50, 55, 60, 65, 70, 75, 80, 85, 90, 95, 100, 105, 110, 115, 120, 125, 130, 135, 140, 145, 150, 155, or 160.

18. The method of claim 17, wherein the predetermined cut-off or the predetermined threshold is a Cyclin E1 IHC H-score of above 50.

19. The method of claim 17, wherein the predetermined cut-off or the predetermined threshold is a Cyclin E1 IHC H-score of above 125.

20. The method of any one of claims 1 to 19, wherein the predetermined Cyclin 1 status or the Cyclin E1 biomarker level is independent of CCNE1 gene amplification in the subject.

21. The method of any one of claims 1 to 19, wherein the predetermined Cyclin 1 status or the Cyclin E1 biomarker level is accompanied by a CCNE1 gene-amplified status in the subject.

22. The method of claim 20 or claim 21, wherein the CCNE1 gene amplification or the CCNE1 gene-amplified status is measured by a CCNE1 gene copy number.

23. The method of claim 22, wherein the CCNE1 gene-amplified status is a CCNE1 gene copy number of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

24. The method of claim 23, wherein the CCNE1 gene-amplified status is a CCNE1 gene copy number of at least 7.

25. The method of any one of claims 1 to 24, wherein the subject is selected without determining levels and statuses of other oncogenes.

26. The method of claim 25, wherein the other oncogenes are selected from BRCA1, BRCA2, TP53, PKMYT1, and PPP2R1A.

27. The method of any one of claims 1 to 26, wherein the cancer is a solid tumor or a hematologic malignancy.

28. The method of any one of claims 1 to 27, wherein the cancer is a Cyclin E1-driven cancer.

29. The method of any one of claims 1 to 28, wherein the cancer is selected from glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colon cancer, colorectal cancer, endometrial cancer, endometrium cancer, esophagus cancer, eye cancer, gallbladder cancer, gastric cancer, gastrointestinal cancer, Hodgkin lymphoma, Non-Hodgkin lymphoma, hematological tumor, head cancer, hematologic malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, uterine sarcoma, uterine serous carcinoma (USC), uterine CS, vaginal cancer, vulvar cancer, Waldenstrom macroglobulinemia, Wilms tumor, solid tumor, or liquid tumor, HGSOC, invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymona, BRAF mutant metastatic colorectal cancer, uveal melanoma, high-grade serous ovarian, fallopian tube, or primary peritoneal cancer, BRAF V600E-mutated colorectal cancer, platinum-sensitive ovarian cancer, poly(ADP-ribose) polymerase inhibitor (PARPi)-resistant ovarian cancer, platinum-resistant ovarian cancer, platinum-refractory ovarian cancer, advanced pancreatic adenocarcinoma, pancreatic ductal adenocarcinoma, neuroendocrine tumor, neuroendocrine prostate cancer, pancreatic neuroendocrine tumor, small cell lung cancer (SCLC), germ cell cancer, and stromal cancer.

30. The method of any one of claims 1 to 29, wherein the cancer is histologically or cytologically confirmed or the cancer is pathologically confirmed.

31. The method of any one of claims 1 to 30, wherein the cancer is recurrent or persistent.

32. The method of any one of claims 1 to 31, wherein the cancer is metastatic.

33. The method of any one of claims 1 to 32, wherein the cancer is unresectable.

34. The method of any one of claims 1 to 33, wherein the subject has received no more than 1, at least 1, 1, 2, 3, 4, 1 or 2, 1 to 2, 1 to 3, or 1 to 4 prior line(s) of therapy, prior line(s) of therapy in the advanced or metastatic setting, prior line(s) of chemotherapy, prior line(s) of platinum-based chemotherapy, prior regimen(s), or prior therapeutic regimen(s).

35. The method of any one of claims 1 to 34, wherein the cancer is platinum-resistant, platinum-sensitive, or platinum-refractory.

36. The method of any one of claims 1 to 35, wherein the cancer is PARP inhibitor-resistant.

37. The method of any one of claims 1, 2, and 4 to 36, comprising administering an effective dose of Azenosertib, or a pharmaceutically acceptable salt thereof without in combination with a second chemotherapeutic agent, or a pharmaceutically acceptable salt thereof.

38. The method of any one of claims 1 and 4 to 36, comprising administering an effective dose of Azenosertib, or a pharmaceutically acceptable salt thereof in combination with a second chemotherapeutic agent, or a pharmaceutically acceptable salt thereof.

39. The method of any one of claims 3 to 38, wherein the second chemotherapeutic agent is selected from bendamustine, bortezomib, carfilzomib, ixazomib, busulfan, carboplatin, cisplatin, cyclophosphamide, cladribine, paclitaxel, docetaxel, pegylated liposomal doxorubicin (PLD), dexamethasone, doxorubicin, gemcitabine, cytarabine, fludarabine, fluorouracil (5-FU), irinotecan, topotecan, temozolomide, triapine, azacitidine, 5-azacytidine, capecitabine, AraC-FdUMP[10] (CF-10), cladribine, etoposide, decitabine, daunorubicin, doxorubicin, ifosfamide, methotrexate, vincristine, hydroxyurea oxaliplatin, niraparib, encorafenib, and cetuximab, or a pharmaceutically acceptable salt of any of the foregoing.

40. The method of claim 39, wherein the second chemotherapeutic agent is carboplatin, paclitaxel, gemcitabine, or pegylated liposomal doxorubicin (PLD), or a pharmaceutically acceptable salt of any of the foregoing.

41. The method of any one of claims 3 to 36 and 38 to 40, wherein Azenosertib, or a pharmaceutically acceptable salt thereof, and the second chemotherapeutic agent, or a pharmaceutically acceptable salt thereof, are administered concurrently.

42. The method of any one of claims 3 to 36 and 38 to 40, wherein Azenosertib, or a pharmaceutically acceptable salt thereof, and the second chemotherapeutic agent, or a pharmaceutically acceptable salt thereof, are administered sequentially.

43. The method of any one of claims 3 to 36 and 38 to 42, wherein Azenosertib, or a pharmaceutically acceptable salt thereof, and/or the second chemotherapeutic agent, or a pharmaceutically acceptable salt thereof, are administered intermittently.

44. The method of any one of claims 1 to 43, wherein the method comprises a step of selecting the subject having the predetermined Cyclin E1 status or the Cyclin E1 biomarker level above the predetermined threshold.

45. The method of claim 44, wherein the method further comprises first determining the Cyclin E1 status or the Cyclin E1 biomarker level prior to the selecting step.

46. The method of any one of claims 1 to 45, wherein the method results in a subject overall response rate (ORR) at or greater than 10%, 15%, 20%, 25%, 30%, 35%, 40%, 45%, or 50%.

47. The method of claim 46, wherein the overall response rate is measured by complete response (CR), partial response (PR), CA-125 50% response, or a combination thereof.

48. The method of claim 47 or claim 48, wherein the method results in a subject median progression-free survival (mPFS) of 5 months, 6 months, 7 months, 8 months, 9 months, 10 months, 11 months, 12 months or longer.

49. A method of treating ovarian cancer comprising:
administering to a subject selected to have a predetermined Cyclin E1 status, an effective dose of Azenosertib, or a pharmaceutically acceptable salt thereof, for a treatment cycle, and
optionally administering a second chemotherapeutic agent, or a pharmaceutically acceptable salt thereof one or more times during the treatment cycle.

50. A method of treating ovarian cancer comprising:
administering to a subject selected to have a Cyclin E1 biomarker level above a predetermined threshold, an effective dose of Azenosertib, or a pharmaceutically acceptable salt thereof, for a treatment cycle, and
administering a second chemotherapeutic agent, or a pharmaceutically acceptable salt thereof, one or more times during the treatment cycle.
